(19) United States
(12) Patent Application Publication
(10) Pub. No.: US 2019/0130994 A1
(43) Pub. Date: May 2, 2019

(54) Mass Spectrometric Data Analysis Workflow

(71) Applicant: DiscernDx, Inc., Palo Alto, CA (US)

(72) Inventors: Daniel Ruderman, San Diego, CA (US); Jeffrey Jones, Glendale, CA (US); Ryan Benz, Huntington Beach, CA (US)

(21) Appl. No.: 16/092,434

(22) PCT Filed: Apr. 11, 2017
(86) PCT No.: PCT/US17/27051

§ 371 Date: Oct. 9, 2018

Related U.S. Application Data
(60) Provisional application No. 62/321,098, filed on Apr. 11, 2016.
(60) Provisional application No. 62/321,099, filed on Apr. 11, 2016.
(60) Provisional application No. 62/321,102, filed on Apr. 11, 2016.
(60) Provisional application No. 62/321,104, filed on Apr. 11, 2016.
(60) Provisional application No. 62/321,110, filed on Apr. 11, 2016.

(51) Int. Cl. G16B 15/20

(52) U.S. Cl. CPC G16B 15/20

(57) ABSTRACT

A number of methods and computer systems related to mass spectrometric data analysis are disclosed. Adoption of the disclosure herein facilitates automated, high throughput, rapid analysis of complex datasets such as datasets generated through mass spectrometric analysis, so as to reduce or eliminate the need for oversight in the analysis process while rapidly yielding accurate results.

### CROSS-REFERENCE

This application claims the benefit of U.S. Prov. App. Ser. No. 62/321,098, filed Apr. 11, 2016, which is hereby explicitly incorporated herein by reference in its entirety; this application claims the benefit of U.S. Prov. App. Ser. No. 62/321,099, filed Apr. 11, 2016, which is hereby explicitly incorporated herein by reference in its entirety; this application claims the benefit of U.S. Prov. App. Ser. No. 62/321,102, filed Apr. 11, 2016, which is hereby explicitly incorporated herein by reference in its entirety; this application claims the benefit of U.S. Prov. App. Ser. No. 62/321,104, filed Apr. 11, 2016, which is hereby explicitly incorporated herein by reference in its entirety; and this application claims the benefit of U.S. Prov. App. Ser. No. 62/321,110, filed Apr. 11, 2016, which is hereby explicitly incorporated herein by reference in its entirety.

### BACKGROUND

Mass spectrometric analysis shows promise as a diagnostic tool, however, challenges remain relating to the development of high throughput, automated data analysis workflows.

### SUMMARY OF THE INVENTION

Provided herein are embodiments related to biomarker database generation and use in patient health classification. Disclosed herein are methods for carrying out mass spectrometric output data processing comprising: generating a quantified output of the mass spectrometric output; comparing the quantified output to a reference; and categorizing the quantified output relative to the reference, wherein practice of the method does not require human supervision. Various aspects incorporate at least one of the following elements. Some aspects comprise a second mass spectrometric output received concurrently with said generating a quantified output of the mass spectrometric output of a first reference. In some embodiments, the method is completed in no more than 1, 2, 3, 4, 5, 6, 7, 8, 9, 10, 11, 12, 13, 14, 15, 16, 17, 18, 19, 20, 21, 22, 23, and 24 hours. In some cases, the method is completed in more than 1, 5, 10, 15, 20, 25, 30, 35, 40, 45, 50, 55, and 60 minutes. Alternately or in combination, some aspects comprise obtaining a fluid sample, and subjecting the fluid sample to mass spectrometric analysis, thereby generating a quantified output of the mass spectrometric analysis. The fluid sample is a dried fluid sample in some aspects. Obtaining the dried fluid sample often comprises depositing a sample onto a sample collection backing. In various aspects, separating plasma from whole blood on the backing comprises contacting whole blood to a filter on the backing. In some cases, subjecting the dried fluid sample to mass spectrometric analysis comprises volatilizing the sample. In various aspects, subjecting the dried fluid sample to mass spectrometric analysis comprises subjecting the sample to proteolytic degradation. In some embodiments, the proteolytic degradation comprises enzymatic degradation. In various cases, the enzymatic degradation comprises contacting a sample to at least one of ArgC, AspN, chymotrypsin, GluC, LysC, LysN, trypsin, snake venom diesterase, pectinase, papain, alcanase, neutrase, snailase, cellulase, amylase, and chitinase. In some cases, the enzymatic degradation comprises trypsin degradation. The proteolytic degradation comprises nonenzymatic degradation in some cases. In various embodiments, the nonenzymatic degradation comprises at least one of heat, acidic treatment, and salt treatment. In some aspects, the nonenzymatic degradation comprises contacting a sample to at least one of hydrochloric acid, formic acid, acetic acid, hydroxide bases, cyanogen bromide, 2-nitro-5-thiocyanobenzoate, and hydroxylamine. Generating a quantified output of the mass spectrometric analysis often comprises quantifying no more than one of at least 20, 50, 100, 5000, and 15000 mass points. In various cases, generating a quantified output of the mass spectrometric analysis is completed in no more than 1, 5, 10, 15, 20, 25, 30, 35, 40, 45, 50, 55, and 60 minutes. Generating a quantified output of the mass spectrometric analysis is often automated. In various cases, generating a quantified output of the mass spectrometric analysis comprises generating an adjusted abundance value. Generating a quantified output of the mass spectrometric analysis comprises generating an adjusted mz value, in some aspects. Alternatively or in combination, generating a quantified output of the mass spectrometric analysis comprises performing a convolution operation to reduce pixel-by-pixel noise of the mass spectrometry data; and identifying a plurality of features of the sample, wherein identifying the plurality of features comprises identifying a plurality of peaks of the mass spectrometry data, and determining a respective mz value and a respective LC value for the plurality of peaks. In various aspects, generating a quantified output of the mass spectrometric analysis comprises receiving data for a plurality of identified peaks from mass spectrometry data of the sample; filtering the plurality of identified peaks to provide a filtered set of peaks, the filtering comprising (1) a first filtering process on the data for the plurality of identified peaks, the first filtering process comprising a peak contrast filtering process, and (2) a second filtering process for removing at least one of a spurious peak and a peak corresponding a calibrant analyte; and selecting a subset of peaks from the plurality of peaks, the subset of peaks comprising peaks corresponding to molecular feature isotopic clusters. In various aspects, generating a quantified output of the mass spectrometric analysis comprises receiving mass spectrometry data of the sample, the mass spectrometry data comprising data for a peptide; and determining a metric value indicative of a likelihood of successful sequencing of the peptide. In many cases, generating a quantified output of the mass spectrometric analysis comprises receiving mass spectrometry data of the sample, the mass spectrometry data comprising a molecular mass value of the sample; and determining, using the mass defect histogram library, a mass defect probability for identifying the molecular mass value, wherein the mass defect probability is indicative of a probability the molecular mass value corresponds to a peptide from the sample. In various embodiments, generating a quantified output of the mass spectrometric analysis comprises receiving tandem mass spectrometry data of the sample, the tandem mass spectrometry data comprising respective molecular mass values for a plurality of identified peaks; and determining a metric value indicative of correspondence between the molecular mass values and molecular mass values of known peptides fragments. In some cases, generating a quantified output of the mass spectrometric analysis comprises receiving tandem mass spectrometry data of the sample, the tandem mass spectrometry data comprising respective molecular mass values for a plurality of identified peaks; and determining a metric value indicative of correspondence between the molecular mass values and molecular mass values of known peptides. Generating a quantified output of the mass spectrometric analysis often comprises identifying data features corresponding to the set of targeted mass spectrometric features; determining characteristics comprising mass, charge and elution time for the data features; and calculating deviation between targeted mass spectrometric feature characteristics and data feature characteristic. Generating a quantified output of the mass spectrometric analysis comprises comparing mass spectrometry data to the set of protein modifications and digestion variants; and assessing the frequency of at least one of protein modifications and digestion frequency, in various embodiments. In some aspects, generating a quantified output of the mass spectrometric analysis comprises identifying test peptide signals in a mass spectrometric output. Certain aspects comprise generating a quantified output of the mass spectrometric analysis comprises identifying reference clusters having exactly one feature per sample; assigning an index area derived from the reference clusters; and mapping nonreference clusters onto the index area. In some embodiments, generating a quantified output of the mass spectrometric analysis comprises identifying features having common m/z ratios across a plurality of samples; aligning said features across a plurality of samples; bringing LC times for said features in line; and clustering said features. Generating a quantified output of the mass spectrometric analysis comprises identifying features having common m/z ratios and common LC times across a plurality of fractions of a sample; assigning to a common cluster features sharing a common m/z ratios and common LC times in adjacent fractions; and discarding said cluster and retain said features when said cluster has at least one of a size above a threshold and an LC time above a threshold, in some cases. Various aspects comprise generating a quantified output of the mass spectrometric analysis comprises choosing a first random subset of fraction outputs; counting the number of unique pieces of information for the first random subset of fraction outputs; choosing a second random subset of fraction outputs; counting the number of unique pieces of information for the second random subset of fraction outputs; and selecting the random subset of fraction outputs having the greater number of unique pieces of information. Generating a quantified output of the mass spectrometric analysis often comprises identifying measured features for said mass spectrometric fraction outputs; calculating average m/z and LC time values for measured features appearing in multiple mass spectrometric fraction outputs; assaying for unidentified features sharing at least one of average m/z and LC time values with said measured features; and assigning at least one of said unidentified features to a cluster of a measured feature, so as to generate at least one inferred mass feature. In some embodiments, generating a quantified output of the mass spectrometric analysis comprises calculating expected LC retention times; calculating standard deviation values of expected LC retention times; comparing expected LC retention times to observed associated LC retention times; and discarding mass spectrometric peptide identification calls for which expected LC retention times differ from observed associated LC retention times by more than standard deviation values. In certain aspects, generating a quantified output of the mass spectrometric analysis comprises identifying features corresponding to common peptides and having differing LC retention times in the plurality of mass spectrometry outputs; applying an LC retention time shift to one of the mass spectrometry outputs so as to bring the differing LC times into closer alignment for the features corresponding to common peptides; applying the LC retention time shift to additional features in proximity to the features corresponding to common peptides in the mass spectrometry output; and discarding mass spectrometric peptide identification calls for which expected LC retention times differ from observed associated LC retention times by more than standard deviation values. In various embodiments, generating a quantified output of the mass spectrometric analysis comprises grouping proteins sharing at least one common peptide; determining a minimum number of proteins per group; and determining a sum for the minimum number of proteins per group for all groups. Generating a quantified output of the mass spectrometric analysis comprises constructing a command line in a format compatible with a given search engine; initiating execution of the search engine; parsing the search engine output; and configuring the output into a standard format, in various aspects. In some cases, generating a quantified output of the mass spectrometric analysis comprises parsing file contents from a memory unit into key-value pairs; read each key-value pair into a standard format; and writing the standard format key-value pairs into an output file. Various aspects comprise generating a quantified output of the mass spectrometric analysis comprises parsing a file into an array of key-value pairs representative of tandem mass spectra and corresponding attributes; obtaining corresponding precursor ion attributes; replacing mass spectrum file values using precursor ion attributes when precursor ion attributes are indicated to be accurate; and configuring the file into a flat format output. Generating a quantified output of the mass spectrometric analysis often comprises receiving a mass spectrometry output having a plurality of unidentified features; including features having a z-value of greater than 1 up to and including 5; clustering included features by retention time to form clusters; de-prioritizing clusters for which verification has been previously performed; selecting a single feature per cluster; and verifying a feature of at least one cluster. In various cases, generating a quantified output of the mass spectrometric analysis comprises generating a processed dataset from one of a plurality of received mass spectrometric output; and incorporating the processed dataset into a processed study dataset. In some aspects, generating a quantified output of the mass spectrometric analysis comprises receiving a first mass spectrometric output and a second mass spectrometric output; performing a quality analysis on the first mass spectrometric output; incorporating the first mass spectrometric output into a processed dataset; performing a quality analysis on the second mass spectrometric output; incorporating the second mass spectrometric output into the processed dataset; wherein performing the quality analysis on the first mass spectrometric output and receiving the second mass spectrometric output are concurrent. In some aspects, generating a quantified output of the mass spectrometric analysis does not comprise human analysis of the mass spectrometric analysis. Generating a quantified output of the mass spectrometric analysis comprises identifying at least 3 reference mass outputs in the mass spectrometric analysis, in various embodiments. In certain aspects generating a quantified output of the mass spectrometric analysis comprises identifying at least 6 reference mass outputs in the mass spectrometric analysis. In various aspects, generating a quantified output of the mass spectrometric analysis comprises identifying at least 10 reference mass outputs in the mass spectrometric analysis. In certain embodiments, generating a quantified output of the mass spectrometric analysis comprises identifying at least 100 reference mass outputs in the mass spectrometric analysis. In some cases, the at least 3 reference mass outputs are introduced to the sample prior to analysis. In various embodiments, the at least 3 reference mass outputs differ from sample mass outputs by known amounts. In certain aspects, the at least 3 reference mass outputs have known amounts. Various aspects comprise comparing reference mass output amounts to sample output amounts. Comparing the quantified output to a reference comprises identifying a subset of the sample mass output, and comparing said subset of the sample mass output to the reference, in certain cases. In some embodiments, the reference comprises at least one sample output of known status for a health category. In various aspects, the reference comprises at least ten sample outputs of known status for a health category. The reference comprises at least ten samples of unknown health status for a health category, in some cases. The reference sometimes comprises predicted values for a health status for a health category. In various cases, the reference comprises samples taken from at least two individuals. In various embodiments, the reference comprises samples taken from at least two time points. The reference often comprises a sample taken from a source common to the sample. Categorizing the quantified output relative to the reference comprises assigning a health category status to an individual source of the sample, in some cases. In some aspects, categorizing the quantified output relative to the reference comprises assigning the reference health category status to an individual source of the sample. Categorizing the quantified output relative to the reference often comprises assigning the reference health category status to an individual source of the sample. In some cases, categorizing the quantified output relative to the reference comprises assigning a percentage value to an individual source of the sample. In various aspects, the percentage value represents the position of the sample relative to the reference.

Disclosed herein are methods comprising: obtaining a biological sample; subjecting the biological sample to mass spectrometric analysis; generating a quantified output of the mass spectrometric analysis; comparing the quantified output to a reference; and categorizing the quantified output relative to the reference, wherein the method does not comprise human supervision.

Disclosed herein are methods comprising: obtaining a biological sample; subjecting the biological sample to mass spectrometric analysis; generating a quantified output of the mass spectrometric analysis; comparing the quantified output to a reference; and categorizing the quantified output relative to the reference, wherein the method is automated.

Disclosed herein are methods comprising: obtaining a biological sample; subjecting the biological sample to mass spectrometric analysis; generating a quantified output of the mass spectrometric analysis; comparing the quantified output to a reference; and categorizing the quantified output relative to the reference, wherein the generating, comparing and categorizing are completed in no more than 30 minutes. Various aspects incorporate at least one of the following elements. In some aspects, the generating, comparing and categorizing are completed in no more than 15 minutes, or no more than 10, 5, or 1 minute.

Disclosed herein are computer systems for mass spectrometry analysis of a sample, comprising: a processor; and a memory to store a computer program, the computer program comprising instructions for: receiving raw mass spectrometry data of the sample, the raw mass spectrometry data comprising corresponding abundance values and corresponding mz values for features contained in the sample; performing at least one of (1) generating an adjusted abundance value, and (2) generating an adjusted mz value; and generating a text based data file using the raw mass spectrometry data. Various aspects incorporate at least one of the following elements. In various aspects, the computer program further comprises instructions for: determining a plurality of abundance values from the raw mass spectrometry data; generating a corresponding adjusted abundance value from each abundance value of the plurality of abundance values, wherein generating the adjusted abundance value comprises setting an abundance value to zero if the abundance value is less than a predetermined abundance value threshold. In some cases, the computer program further comprises instructions for: determining a plurality of mz values from the raw mass spectrometry data; generating a corresponding adjusted mz value from each mz value of the plurality of mz values, wherein generating the adjusted mz value comprises setting a mz value to a predetermined mz value. In various cases, receiving the raw mass spectrometry data comprises receiving raw mass spectrometry data from one mass scan of a sample. In some embodiments, receiving the raw mass spectrometry data comprises receiving raw mass spectrometry data from at least two mass scans of a sample. The computer program further comprises instructions for storing pairs of adjusted abundance values and adjusted mz values, in some cases.

Disclosed herein are computer systems for mass spectrometry analysis of a sample, comprising: a processor; and a memory to store a computer program, the computer program comprising instructions for: receiving a text based mass spectrometry data of the sample, the text based mass spectrometry data comprising mass spectrometry data from a plurality of mass scans; and generating an image pixel representation of the mass spectrometry data for the plurality of mass scans, the image pixel representation comprising a plurality of pixels, wherein generating the image pixel representation comprises determining a value of each pixel of the plurality of pixels, and wherein determining the value of each pixel comprises accumulating abundance values across the plurality of scans for each pixel. Various aspects incorporate at least one of the following elements. In some cases, computer program further comprises instructions for mapping each mz value of the mass spectrometry data to a corresponding first value between 0 and 1. In various aspects, the computer program further comprises instructions for mapping each LC value of the mass spectrometry data to a corresponding second value between 0 and 1. Generating the image pixel representation often comprises generating the plurality of pixels comprising a width of W pixels and a height of H pixels. In some cases, accumulating the abundances comprises performing an interpolation. In various aspects, accumulating the abundances comprises performing a linear interpolation. Accumulating the abundances comprises performing a nonlinear interpolation, in some embodiments. In various cases, accumulating the abundances comprises performing an integration.

Disclosed herein are computer systems for mass spectrometry analysis of a sample, comprising: a processor; and a memory to store a computer program, the computer program comprising instructions for: receiving mass spectrometry data of the sample; performing a convolution operation to reduce pixel-by-pixel noise of the mass spectrometry data; and identifying a plurality of features of the sample, wherein identifying the plurality of features comprises identifying a plurality of peaks of the mass spectrometry data, and determining a respective mz value and a respective LC value for the plurality of peaks. Various aspects incorporate at least one of the following elements. Identifying the plurality of features comprises determining a respective peak height and a respective peak area for the plurality of peaks in various cases. In some aspects, identifying the plurality of features comprises subjecting the mass spectrometry data to a machine learning analysis. Identifying the plurality of features comprises subjecting the mass spectrometry data to an artificial intelligence analysis in some cases. In various embodiments, identifying the plurality of peaks comprises selecting a peak comprising a height than a predetermined threshold, and greater than corresponding heights of at least eight adjacent peaks.

Disclosed herein are computer systems configured for mass spectrometry analysis of a sample, comprising: a processor; and a memory to store a computer program, the computer program comprising instructions for: receiving data for a plurality of identified peaks from mass spectrometry data of the sample; filtering the plurality of identified peaks to provide a filtered set of peaks, the filtering comprising (1) a first filtering process on the data for the plurality of identified peaks, the first filtering process comprising a peak contrast filtering process, and (2) a second filtering process for removing at least one of a spurious peak and a peak corresponding a calibrant analyte; and selecting a subset of peaks from the plurality of peaks, the subset of peaks comprising peaks corresponding to molecular feature isotopic clusters. Various aspects incorporate at least one of the following elements. In some cases, the data for the plurality of identified peaks comprises a respective mz value, a respective LC value, a respective abundance value, and a respective chromatographic value for each of the plurality of identified peaks. In various aspects, the respective chromatographic value for the plurality of identified peak comprises a peak width value. Selecting the subset of peaks comprises providing a respective mz value, a respective LC value, a respective peak height value, a respective peak area value, and a respective chromatographic value for each of the subset of peaks in some embodiments. The computer program in some aspects further comprises instructions for calibrating each of the plurality of filtered peaks to provide a plurality of calibrated peaks, the calibrating comprising calibrating respective mz values for each of the plurality of filtered peaks. The computer program further comprises instructions for generating a 2-dimensional matrix to bin the plurality of calibrated peaks to provide a plurality of binned peaks in some cases. In various embodiments, the computer program further comprises instructions for combining the plurality of binned peaks to form the isotopic clusters. In some aspects, the computer program further comprises instructions to mapping the isotopic clusters to identified molecular features.

Disclosed herein are computer systems configured for mass spectrometry analysis of a sample, comprising: a processor; and a memory to store a computer program, the computer program comprising instructions for: receiving mass spectrometry data of the sample, the mass spectrometry data comprising data for a peptide; and determining a metric value indicative of a likelihood of successful sequence determination for the peptide. In various cases, receiving the mass spectrometry data comprises receiving mass spectrometry data for an isotopic envelope of a feature, an estimated mz value corresponding to the feature and a charge state corresponding to the feature.

Disclosed herein are computer systems configured for mass spectrometry analysis of a sample, comprising: a processor; and a memory to store a computer program, the computer program comprising instructions for: providing a mass defect histogram library comprising a mass defect histogram for each of a plurality of neutral mass values; receiving mass spectrometry data of the sample, the mass spectrometry data comprising a molecular mass value of the sample; and determining, using the mass defect histogram library, a mass defect probability for identifying the molecular mass value, wherein the mass defect probability is indicative of a probability the molecular mass value corresponds to a peptide from the sample. Various aspects incorporate at least one of the following elements. In some aspects, the computer program further comprises instructions for identifying the peptide using the mass defect histogram library. Providing the mass defect histogram library comprises generating the mass defect histogram library using predetermined neutral mass values in various cases. In some aspects, the computer program further comprises instructions for receiving a library comprising a plurality of neutral mass values corresponding to a plurality of known peptides. The computer program further comprises instructions for normalizing each of the plurality of neutral mass values corresponding to the plurality of known peptides in some embodiments. In various aspects, the computer program further comprises instructions for receiving a library comprising a plurality of neutral mass values corresponding to a plurality of predicted peptides. In some cases, the computer program further comprises instructions for normalizing each of the plurality of neutral mass values corresponding to the plurality of predicted peptides.

Disclosed herein are computer systems configured for mass spectrometry analysis of a sample, comprising: a processor; and a memory to store a computer program, the computer program comprising instructions for: receiving tandem mass spectrometry data of the sample, the tandem mass spectrometry data comprising respective molecular mass values for a plurality of identified peaks; and determining a metric value indicative of correspondence between the molecular mass values and molecular mass values of known peptides fragments. Various aspects incorporate at least one of the following elements. In some embodiments, receiving the tandem mass spectrometry data comprises receiving: (1) a mass probability value, (2) a mz value, and (3) a z value. In various aspects, the computer program further comprises instructions for: receiving a peptide mass value library comprising a plurality of mass peptide values; determining a neutral mass value; and determining a defect probability value. In some cases, determining the defect probability value comprises interpolating the plurality of mass peptide values using the neutral mass value.

Disclosed herein are computer systems configured for mass spectrometry analysis of a sample, comprising: a processor; and a memory to store a computer program, the computer program comprising instructions for: receiving tandem mass spectrometry data of the sample, the tandem mass spectrometry data comprising respective molecular mass values for a plurality of identified peaks; and determining a metric value indicative of correspondence between the molecular mass values and molecular mass values of known peptides. Various aspects incorporate at least one of the following elements. In various cases, receiving the tandem mass spectrometry data comprises receiving both a respective mz value and a respective abundance value for each of the plurality of identified peaks. Determining the metric value often comprises determining a weighted average. In some aspects, determining the weighted average comprises determining the weighted average based on respective abundance values for the plurality of identified peaks.

Disclosed herein are computer systems configured to identify mass spectrometry output feature characteristics, comprising: a memory unit configured to receive a set of targeted mass spectrometric features having characteristics comprising mass, charge and elution time; a computation unit configured to identify data features corresponding to the set of targeted mass spectrometric features; to determine characteristics comprising mass, charge and elution time for the data features; to calculate deviation between targeted mass spectrometric feature characteristics and data feature characteristic; an output unit configured to provide mass spectrometric information comprising at least one of neutral mass, charge state, observed elution time, and deviation. Various aspects incorporate at least one of the following elements. In various aspects, said characteristics comprise abundance. Said characteristics often comprise intensity.

Disclosed herein are computer systems configured to assess protein mass spectrometry input status, comprising: a memory unit configured to receive a set of protein modifications and digestion variants; a computation unit configured to compare mass spectrometry data to the set of protein modifications and digestion variants; and to assess the frequency of protein modifications; and an output unit configured to report an assessment of protein modifications.

Disclosed herein are computer systems configured to assess mass spectrometry apparatus performance, comprising: a memory unit configured to receive performance parameters for a set of test analyte signals; a computation unit configured to identify test analyte signals in a mass spectrometric output; and assess difference between said signals and said performance parameters; and an output unit configured to provide assessment of the difference between said signals and said performance parameters. Various aspects incorporate at least one of the following elements. In some aspects, the test peptides are selected from the list of peptides in table 3. In various cases, the analyte signals comprise peptide signals corresponding to test peptide accumulation levels. In some embodiments, the analyte signals comprise poly-leucine peptide signals. In some cases, the analyte signals comprise poly-glycine peptide signals. Alternatively, or in combination, the apparatus performance is assessed as to at least one of mass accuracy, LC retention time, LC peak shape, and abundance measurement. In various aspects, the apparatus performance is assessed as to at least one of number of detected peptides, relative change in number of features, maximum abundance error, overall mean abundance shift; standard deviation in abundance shift; maximum m/z deviation; maximum peptide retention time; and maximum peptide chromatographic full-width half maximum.

Disclosed herein are computer systems configured to normalize mass spectrometric peak areas, comprising: a memory unit configured to receive a set of extracted mass spectrometry peak areas; a computation unit configured to identify reference clusters having exactly one feature per sample; to assign an index area derived from the reference clusters; and to map nonreference clusters onto the index area; and an output unit configured to provide corrected peak area outputs.

Disclosed herein are computer systems configured to identify common features of mass spectrometric output across a plurality of samples, comprising: a memory unit configured to receive a set of mass spectrometric outputs; a computation unit configured to identify features having common m/z ratios across a plurality of samples; to align said features across a plurality of samples; to bring LC times for said features in line; and to cluster said features an output unit configured to provide identification of at least one feature common to at least two members of the set of mass spectrometric outputs. In some aspects, being configured to align said features across a plurality of samples comprises being configured to apply a nonlinear retention time warping procedure.

Disclosed herein are computer systems configured to cluster peptide features appearing in a plurality of mass spectrometry fractions, comprising: a memory unit configured to receive a set of mass spectrometric outputs; a computation unit configured to identify features having common m/z ratios and common LC times across a plurality of fractions of a sample; to assign to a common cluster features sharing a common m/z ratios and common LC times in adjacent fractions; and to discard said cluster and retain said features when said cluster has at least one of a size above a threshold and an LC time above a threshold; and an output unit configured to provide cluster identification for a plurality of feature clusters. In some cases, said size has a threshold of 75 ppm and said LC time of at least 50 seconds.

Disclosed herein are computer systems configured to rank mass spectrometry fractions according to information content, comprising: a memory unit configured to receive a set of mass spectrometric fraction outputs; a computation unit configured to choose a first random subset of fraction outputs; to count the number of unique pieces of information for the first random subset of fraction outputs; to choose a second random subset of fraction outputs; to count the number of unique pieces of information for the second random subset of fraction outputs; and to select the random subset of fraction outputs having the greater number of unique pieces of information; and an output unit configured to provide fraction subset information correlated to number of unique pieces of information.

Disclosed herein are computer systems configured to re-extract peptide features appearing in a mass spectrometry output, comprising: a memory unit configured to receive a set of mass spectrometric outputs and to store scoring information for measured features for said mass spectrometric fraction outputs; a computation unit configured to identify measured features for said mass spectrometric outputs; to calculate average m/z and LC time values for measured features appearing in multiple mass spectrometric outputs; to assay for unidentified features sharing at least one of average m/z and LC time values with said measured features; and assigning at least one of said unidentified features to a cluster of a measured feature, so as to generate at least one inferred mass feature; and an output unit configured to provide said measured features and said at least one inferred mass feature observations.

Disclosed herein are computer systems configured to filter inconsistent peptide identification calls, comprising: a memory unit configured to receive a set of mass spectrometric peptide identification calls and associated mass spectrometric LC retention times; a computation unit configured to calculate expected LC retention times; to calculate standard deviation values of expected LC retention times; to compare expected LC retention times to observed associated LC retention times; and to discard mass spectrometric peptide identification calls for which expected LC retention times differ from observed associated LC retention times by more than standard deviation values; and an output unit configured to provide filtered peptide identification calls.

Disclosed herein are computer systems configured to adjust retention times so as to align fragments sharing m/z ratios, comprising a memory unit configured to receive a set of mass spectrometric peptide identification calls and associated mass spectrometric LC retention times for a plurality of mass spectrometry outputs; a computation unit configured to identify features corresponding to common peptides and having differing LC retention times in the plurality of mass spectrometry outputs; to apply an LC retention time shift to one of the mass spectrometry outputs so as to bring the differing LC times into closer alignment for the features corresponding to common peptides; to apply the LC retention time shift to additional features in proximity to the features corresponding to common peptides in the mass spectrometry output; and to discard mass spectrometric peptide identification calls for which expected LC retention times differ from observed associated LC retention times by more than standard deviation values; and an output unit configured to provide a retention time adjusted mass spectrometry output.

Disclosed herein are computer systems configured to calculate a minimum assignable protein count for a mass spectrometric output, the computer system comprising: a memory unit configured to receive a list of identified peptides in a mass spectroscopy output, and a mapping of said identified peptides to all proteins that contain said peptides; a computation unit configured to group proteins sharing at least one common peptide; to determine a minimum number of proteins per group; and to determine a sum for the minimum number of proteins per group for all groups; an output unit configured to provide a minimum number of proteins consistent with the list of identified peptides.

Disclosed herein are computer systems configured to maintain uniform proteomic peptide assignment across peptide analysis platforms, the system comprising: a memory unit configured to receive proteomic peptide assignments in a standard format; and a computation unit configured to construct a command line in a format compatible with a given search engine; initiate execution of the search engine; parse the search engine output; and configure the output into a standard format. Various aspects incorporate at least one of the following elements. In some cases, the computation unit is configured to run a relational database Object operation. In some aspects, the standard configuration comprises at least one parameter selected from a list consisting of precursor ion max mass error, fragment ion max mass error, rank, expectation value, score, processing threads, fasta database and post-translational modifications.

Disclosed herein are computer systems configured to extract tandem mass spectra and assign individual headers with specific spectrum information, comprising: a memory unit comprised to receive mass spectra information; a computation unit configured to parse file contents from the memory unit into key-value pairs; read each key-value pair into a standard format; and write the standard format key-value pairs into an output file. In some embodiments, the key-value pairs comprise at least one of DATA FILE, EXPERIMENT NO, LCMS SCAN NO, LCMS LCTIME, OBSERVED MZ, OBSERVED Z, TANDEM LCMS MAX ABUNDANCE, TANDEM LCMS PRECURSOR ABUNDANCE, TANDEM LCMS SNR, and LCMS SCAN MGF NO.

Disclosed herein are computer systems configured to compute a tandem mass spectra correction, comprising: a memory unit configured to receive a proteomics mass spectrum file; and a computation unit configured to parse the file into an array of key-value pairs representative of tandem mass spectra and corresponding attributes; to obtain corresponding precursor ion attributes; to replace mass spectrum file values using precursor ion attributes when precursor ion attributes are indicated to be accurate; and configure the file into a flat format output.

Disclosed herein are computer systems configured to compute a false discovery rate for feature assignments, comprising: a memory unit configured to receive a list of proteomics search engine results comprising feature assignments; a computation unit configured to assess the list relative to randomly generated lists and assign key-valued pairs to the feature assignments; and an output unit configured to provide a measure of statistical confidence for the feature assignments. In some cases, the computation unit is configured to compute an expectation value for a given false discovery rate using Benjamini-Hochberg-Yekutieli computation.

Disclosed herein are methods of mass spectrometry feature verification selection, comprising receiving a mass spectrometry output having a plurality of unidentified features; including features having a z-value of greater than 1 up to and including 50; clustering included features by retention time to form clusters; de-prioritizing clusters for which verification has been previously performed; selecting a single feature per cluster; and verifying a feature of at least one cluster. Various aspects incorporate at least one of the following elements. In some aspects, a cluster having an identification score of greater than a lowest expected valid score is de-prioritized. A cluster having low abundance features relative to other clusters is de-prioritized in some embodiments. In some cases, selecting comprises prioritizing a cluster having all three of a ms1p of greater than 0.33, an abundance value of greater than a signal to noise ratio of 1/10, and a low mass contamination and well ratio of less than 1. Selecting comprises prioritizing a cluster having at least two of a ms1p of greater than 0.33, an abundance value of greater than 2000, and a low mass contamination and well ratio of less than 1 in some embodiments. In various aspects, selecting comprises prioritizing a cluster having at least one of a ms1p of greater than 0.33, an abundance value of greater than 2000, and a low mass contamination and well ratio of less than 1. Selecting comprises prioritizing a feature having a z=2 unless another feature has greater than twice its abundance in some aspects. In various embodiments, selecting comprises selecting 1 feature per time interval of the mass spectrometric output. The time interval is often no greater than 2 seconds. In some cases, the time interval is about 1.75 seconds. In certain aspects, the time interval is 1.75 seconds.

Disclosed herein are methods of sequential mass spectrometric data analysis, comprising receiving a first mass spectrometric output and a second mass spectrometric output; performing a quality analysis on the first mass spectrometric output; incorporating the first mass spectrometric output into a processed dataset; performing a quality analysis on the second mass spectrometric output; incorporating the second mass spectrometric output into the processed dataset; wherein performing the quality analysis on the first mass spectrometric output and receiving the second mass spectrometric output are concurrent.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Some understanding of the features and advantages of the present invention will be obtained by reference to the following detailed description that sets forth illustrative embodiments, in which the principles of the invention are utilized, and the accompanying drawings.

The patent or application file contains at least one drawing executed in color. Copies of this patent or patent application publication with color drawing(s) will be provided by the Office upon request and payment of the necessary fee.

FIG. 1 shows an exemplary mass spectrometry workflow, from sample acquisition to data analysis.

FIG. 2 shows an example of LC time abundance integration;

FIG. 3 shows an example of an isotopic filtering and deconvolution process work flow;

FIG. 4 shows a molecular weight histogram of a distribution of neutral mass molecular weights from known human peptides;

FIG. 5 shows an expanded view of a portion of the peptide molecular weight histogram of FIG. 4, showing discrete populations for each nominal mass;

FIG. 6 illustrates an example of a set of molecular features;

FIG. 7 illustrates an example of a restricted search space;

FIG. 8 illustrates an example of the application of a restricted search space to a population of molecular features;

FIG. 9 shows an example of the restricted search spaces and their position relative to the molecular features after one or more iterations of this process;

FIG. 10 shows an example of a QC block worklist and a sample block worklist;

FIG. 11 shows an example of a process flow chart of a feature re-extraction process;

FIG. 12 shows an exemplary Noviplex DBS plasma card;

FIG. 13 shows mass spectrometry output graphs resulting from samples subjected to mass spectrometry runs;

FIG. 14 shows graphs illustrating within-card and between-card coefficients of variation (CV) calculated on 64,667 features;

FIG. 15 shows graphs illustrating within-card and between-card coefficients of variation (CV) calculated on 65,795 features;

FIG. 16 shows a graph illustrating between-card coefficient of variation (CV) calculated on 55,939 features;

FIG. 17 shows a graph of the normalized instrument response versus against measured endogenous plasma concentration;

FIG. 18 shows a graph of the normalized instrument response versus the protein concentration rank;

FIG. 19 shows endogenous plasma gelsolin levels measured using two peptides;

FIG. 20 shows a graph illustrating sex prediction results for the sample of origin;

FIG. 21 shows a graph illustrating race prediction results for the sample of origin;

FIG. 22 shows a graph illustrating an example of prediction results of colorectal cancer (CRC) status for the sample of origin;

FIG. 23 shows a graph illustrating another example of prediction results of colorectal cancer (CRC) status for the sample of origin;

FIG. 24 shows a graph illustrating an example of prediction results of prediction of coronary artery disease (CAD) status for the sample of origin;

FIG. 25 shows two graphs of an LC gradient (left panel) and an optimized gradient (right panel;

FIG. 26 shows a mass spectrometric analysis of a 30 minute gradient (left panel) and a 10 minute gradient (right panel);

FIG. 27 shows various sources of biomarker data;

FIG. 28 shows an exemplary tube for collecting breath and mass spectrometry analysis of VOCs from a breath sample;

FIG. 29 shows an exemplary data collection scheme of data;

FIG. 30A shows output data of a mass spectrometric analysis;

FIG. 30B shows output data as in FIG. 30A with an overlay of positions of added heavy labeled markers; and

FIG. 31 shows results of a representative list of 16 markers.

FIG. 32 shows a comparison of batch and iterative data processing workflows.

### DETAILED DESCRIPTION

Disclosed herein are methods and computer systems related to mass spectrometric data workflows. Methods and computer systems herein facilitate the rapid, accurate, automated analysis of data from samples subjected to mass spectrometry analysis.

In particular, methods and computer systems herein facilitate the analysis of raw mass spectrometric output, such as digital images indicating mass spectrometric item mass, time of flight and abundance.

In some alternate approaches, analysis of data output is a bottle-neck in mass spectrometric workflows, both temporally and statistically. Statistically, mass spectrometric analysis is often a source of error introduction, as spot mis-callings, overlapping spots, variation in distance travelled by mass features between runs, and variation in sample input processing all lead to an overestimation of sample variation.

Many alternate methods address these challenges by increasing operator oversight at these steps, so as to reduce error associated with automated data processing. However, operator oversight introduces substantial time delays in data processing, and is not without error.

Disclosed herein are a number of methods and computer systems configured to execute these methods, such that a number of steps in the mass spectrometric data processing pipeline are executed more efficiently, more quickly, and with less error an without operator supervision. Employment of any of these methods or computer systems, individually or in combination, leads to improvements in mass spectrometric workflow, as measured by time, accuracy, and extent of operator supervision required. In some cases, results are generated in real time comparable to that of data input, such that adjustments can be made to a particular workflow as indicated by initial data output.

Through practice of the methods or employment of the computer systems as disclosed herein, mass spectrometric results are obtained in less than one day, for example no more than 8 hours, no more than 6 hours, no more than 4 hours, no more than 2 hours, no more than 1 hour, no more than 30 minutes, no more than 15 minutes, no more than 10 minutes, no more than 5 minutes, or in some cases no more than 4, 3, 2, or 1 minute. Alternately or in combination, raw mass spectrometric data analysis is performed in no more than 1 hour, no more than 45 minutes, no more than 30 minutes, no more than 15 minutes, no more than 10 minute, or no more than 9, 8, 7, 6, 5, 4, 3, 2, 1, or less than one minute.

One or more methods described herein comprise mass spectrometry data analysis, for example processing of data generated using mass spectrometry tools to provide desired analysis of a sample within a reduced time, such as compared to existing analysis methods. Analysis of mass spectrometry measurements performed according to one or more methods described herein can be completed in no more than 8 hours, 4 hours, 2 hours, 1 hour, 30 minutes, 20 minutes, 15 minutes, 10 minutes 5 minutes 1 minute or 30 seconds. The increased speed of analysis as provided herein can enable providing same day turnaround of sample analysis, for example enabling same day diagnosis of various conditions. Increased speed of analysis as provided herein can enable providing same hour turnaround of sample analysis. In some cases, data analysis occurs in no more than 1 minute. For example, a duration of time from providing raw data of a sample generated using a mass spectrometry tool to providing desired analysis of the raw data can be no more than 8 hours, 4 hours, 2 hours, 1 hour, 30 minutes, 20 minutes, 15 minutes, 10 minutes 5 minutes 1 minute or 30 seconds.

The analysis of the raw data can comprise generating a quantified output of the mass spectrometric analysis, comparing the quantified output to a reference, and categorizing the quantified output relative to the reference. The generating a quantified output of the mass spectrometric analysis can be completed in no more than 8 hours, 4 hours, 2 hours, 1 hour, 30 minutes, 20 minutes, 15 minutes, 10 minutes 5 minutes 1 minute or 30 seconds. In some cases, generating a quantified output of the mass spectrometric analysis and comparing the quantified output to a reference can be completed in no more than 8 hours, 4 hours, 2 hours, 1 hour, 30 minutes, 20 minutes, 15 minutes, 10 minutes 5 minutes 1 minute or 30 seconds. In some cases, generating a quantified output of the mass spectrometric analysis, comparing the quantified output to a reference, and categorizing the quantified output relative to the reference can be completed in no more than 8 hours, 4 hours, 2 hours, 1 hour, 30 minutes, 20 minutes, 15 minutes, 10 minutes 5 minutes 1 minute or 30 seconds.

In some cases, analysis of the raw data can be completed without or substantially without human intervention, such as without human analysis. For example, one or more of generating a quantified output of the mass spectrometric analysis, comparing the quantified output to a reference, and categorizing the quantified output relative to the reference can be completed without or substantially without human intervention. Analysis of the raw data can proceed to completion without or substantially to provide a desired output. In some cases, the generating a quantified output of the mass spectrometric analysis can be completed without or substantially without human intervention. For example, the raw data can be provided to a computer system comprising a processor and an associated memory configured to store instructions for executing one or more processes described herein, and the processor can execute the stored instructions using the input raw data to provide desired analysis of the input raw data without or substantially without further human intervention. A user may provide the raw data. Additionally or alternatively, the raw data may be provided automatically, for example by one or more mass spectrometry tools. For example, mass spectrometry raw data of one or more samples can be provided from the mass spectrometry tool to a computer system configured to perform one or more processes described herein, in response to a request instruction and/or automatically after completion of mass spectrometry measurements. A duration of time from provision of the raw input data to receiving the desired output can be no more than one or more periods described herein.

In some cases, a duration of time from receipt of an image file generated using raw mass spectrometry data to providing a desired output after completion of analysis of the mass spectrometry data can be no more than 8 hours, 4 hours, 2 hours, 1 hour, 30 minutes, 20 minutes, 15 minutes, 10 minutes 5 minutes 1 minute or 30 seconds. In some embodiments, one or more processes described herein can be completed within 8 hours, 4 hours, 2 hours, 1 hour, 30 minutes, 20 minutes, 15 minutes, 10 minutes 5 minutes 1 minute or 30 seconds.

Desired analysis of a sample can comprise providing a listing of identified analytes in the sample, such as detected proteins in the sample. In some cases, desired analysis can comprise providing a list of proteins present in the sample and one or more characteristics of the detected proteins. In some embodiments, desired analysis comprises analysis of raw data from many samples. In some embodiments, desired analysis comprises analysis of raw data generated by multiple mass spectrometry tools. Desired analysis can comprise quantifying at least 20 mass points, at least 50 mass points at least 100 mass points, at least 5,000 mass points, or at least 15,000 mass points. Desired analysis can comprise identifying at least 3 reference mass outputs, at least 6 reference mass outputs, at least 10 reference mass outputs, or at least 100 reference mass outputs.

A sample as described herein can comprise one or more of a fluid sample and a dry sample. The dry sample may comprise a dried fluid sample, such as a dried bloodspot.

Mass spectrometry measurements can be generated using various types of mass spectrometry tools, including for example liquid chromatography mass spectrometry (LCMS), and/or tandem mass spectrometry.

Through practice of the methods or employment of the computer systems as disclosed herein, mass spectrometric results are obtained through an approach that is automated, up to and including fully automated, such that operator intervention is not required between sample input and final data and computational assessment conclusion output. Results are obtained in some cases in real time, such that adjustments to sample collection, sample processing and data output can be made in light of results from earlier samples prior to completion of sample input or sample analysis, thereby facilitating workflow correction or modification, or sample assessment, without loss of time and reagent associated with running entire sample batches prior to output generation.

Some embodiments comprise methods of automated mass spectrometry analysis and computer systems configured to carry out LCMS data extraction. Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute. Method described herein may be practiced as part of an automated workflow, without human oversight, and in some cases in on a time scale limited by computational capacity.

Extraction of relevant information from data generated by a mass spectrometry tool can include conversion of the raw data into image files. The image files may then be processed using one or more methods described herein, so as to extract desired information from the image files within a desired duration of time. In some cases, extraction of desired information from raw data generated by a mass spectrometry tool can be completed in no more than 8 hours, 4 hours, 2 hours, 1 hour, 30 minutes, 20 minutes, 15 minutes, 10 minutes 5 minutes 1 minute or 30 seconds. For example, a duration of time from receipt of the raw data to providing a desired output, such as providing a listing of proteins identified in a sample, can be no more than 8 hours, 4 hours, 2 hours, 1 hour, 30 minutes, 20 minutes, 15 minutes, 10 minutes 5 minutes 1 minute or 30 seconds.

In some embodiments, provided herein are methods for converting raw data generated based on measurements taken by a mass spectroscopy tool into a format which can be converted into an image file. For example, the raw data conversion process can comprise converting the raw data into a text format. The text based file can then be converted into an image file, and the image file can be further processed to extract the desired information. Mass spectrometry measurements of a sample injection made by a mass spectrometry tool can be provided in a raw data format, the raw data for example provided as an output from the mass spectrometry tool. The raw data output from the mass spectrometry can be converted to a text file. Conversion of raw data from a mass spectrometry tool can be converted into a text format can be performed as described herein, such as to generate a text based MS1 data and/or a text based MS2 data.

Raw data can be provided, for example through .Net Application Programming Interfaces (API) run on the Windows platform. The API's can allow the extraction of MS1 and MS2 data from the raw data. The API's can also allow the extraction of other information about a sample injection through the creation of programs that employ the API. Data can be converted to a text-based data file format that enables multiple technologies not generally compatible with the .NET platform to access the data.

The raw data conversion process can comprise a lossy process. As used herein, lossy data conversions refer to converting data from a first data format into a second different data format where a difference exists between information contained between the first data format and the second different data format, such as due to discarded information and/or use of approximations. Lossy data conversions can result in a loss of information in the different data format to facilitate ease and/or speed of the conversion, for example to provide extraction of desired information from the first data format while facilitating increased processing speed.

As an example, raw data can be converted into a text-based data file (e.g., an “apims1” file) containing mass spectrometry spectral data (e.g., MS1 spectral data) for a given injection on a scan-by-scan basis. The text-based data file can be provided as output of the raw data conversion process.

The raw data conversion process can receive as an input a raw data file for a given injection. The raw data file can be accessed from a location such as a “.d” file directory. The raw data conversion process can utilize one or more constants during its execution. The raw data conversion process can use a first constant determining an abundance threshold (e.g., “ABUNDANCE THRESHOLD”). The first constant can be set equal to 100, although other numbers may be consistent with the operation of various embodiments of the process. In some embodiments, the first constant can be set equal to at least 10, 20, 30, 40, 50, 60, 70, 80, 90, 100, 110, 120, 130, 140, 150, 160, 170, 180, 190, 200, or 250. In some embodiments, the first constant can be set equal to no more than 10, 20, 30, 40, 50, 60, 70, 80, 90, 100, 110, 120, 130, 140, 150, 160, 170, 180, 190, 200, or 250. The raw data conversion process can use a second constant, for example a rounding value (e.g., “DELTA_MZ”). The second constant can be set equal to 0.0001, although other numbers are consistent with the operation of various embodiments of the process. In some embodiments, the second constant can be set equal to at least 0.1, 0.01, 0.001, 0.0001, 0.00001, or 0.000001. In some embodiments, the second constant can be set equal to no more than 0.1, 0.01, 0.001, 0.0001, 0.00001, or 0.000001.

An example of a raw data conversion process work-flow is as follows. Each of a plurality of scans taken by a mass spectrometry tool in acquiring data of a sample injection (e.g., each of a plurality of MS1 scans) can be processed. The output can be performed time-sequentially as performed in the mass spectrometry.

First, mz values (mass-to-charge values) and their corresponding abundance values can be extracted from the raw data for each scan performed by the mass spectrometry tool. For example, pairs of corresponding mz values and abundance values, for example pairs of (mz, abundance), can be extracted. mz (mass-to-charge) and abundance values can be extracted using the API for each MS1 scan. Second, each abundance value can be compared to an abundance threshold value. Any abundance values lower than the abundance threshold value can be set to zero. For example, abundance values in the data file for each scan can be compared with the ABUNDANCE THRESHOLD constant and abundance values lower than the ABUNDANCE THRESHOLD can be set to zero. Setting the abundance values which are less than a threshold value to zero can be a lossy step that results in some loss or change of information from the raw data file but can reduce file size and/or enhance the speed of downstream calculations.

Third, the mz values for a given scan are then rounded to size DELTA_MZ. Rounding mz values to DELTA_MZ can enable use of an array index to store the mz information, for example instead of storing the mz values directly. Although rounding of the mz values can result in information loss, the rounding can enable quicker storage of data and/or data storage using less memory. Fourth, each of the pairs of rounded mz values and thresholded abundance values can be stored for each scan. The rounded mz values and thresholded abundance values can be provided as an output API data file (e.g., an “apims1” file) as mass spectrometry spectral data for a sample injection, for example as MS1 spectral data for a given injection on a scan-by-scan basis.

As described herein, raw data can be converted to text based format for conversion to an image based file. Conversion of the text based file to an image file can comprise a rasterization process. Rasterization comprises generating an image file comprising pixels. The rasterization of mass spectrometry data, such as MS1 data, can provide images for which further processing can be performed using one or more other processes described herein so as to generate the desired output, such as a listing of identified proteins from a sample. The rasterization process can utilize data extracted from a text based data file (e.g., “apims1” file) and output a raster image such as, for example, a pixel representation of the data present in the text based data file. One or more processes, such as a peak detection process described herein (e.g., peak picker), can receive as an input the image data, to generate a list of identified peaks in the data. The one or more processes can treat mass spectrometry data, such as MS1 data, as pixilated images.

An example of an image conversion process for converting text based data to a pixel representation is provided as follows. First, an mz range of interest can be mapped to a first variable (e.g., an “x” variable). The first variable can have a value ranging from 0 to 1, although other ranges can be consistent with the operation of various embodiments of the process. Second, the LC time range of interest can be mapped to a second variable (e.g. a “y” variable). This second variable can have a value ranging from 0 to 1, although other ranges can be consistent with the operation of various embodiments of the process.

Third, the pixel representation can be set to have a number of horizontal pixels (e.g. “W”) and a number of vertical pixels (e.g. “H”). A width of each pixel can be dx=1/W. A height of each pixel can be dy=1/H.

Fourth, a value for each pixel of the image can be determined. Determining a value for a pixel of the image can comprise accumulating abundance values across the plurality of mass spectrometric scans of an injection sample. For example, a value of a pixel centered at location (x, y) having dimensions (dx, dy) in the image can be determined by accumulating abundances across a plurality of scans. In some cases, accumulating abundance values can comprise performing linear interpolation of the total abundance values within the mz range and performing an integration across the LC time range.

Determining a value of a pixel can comprise a number of steps. A scan whose y position is in the range [y−dy/2,y+dy/2] (e.g. within the pixel's y range) as well as the first scan preceding and the first scan following that time range, can be considered. For each of these scans, the total mass spectrometry abundance (e.g., MS1 abundance) present within the x-range of the pixel (e.g. in the x range [x−dx/2, x+dx/2]) can be determined. The total mass spectrometry abundance can be referred to as the summed abundance value Aᵢ for the i^{th} such scan.

The summed abundance values can be added together according to their interpolated and integrated impact on the pixel, as to linearly interpolate and sum the abundance curve over time within the pixel's rectangular time profile. This can be accomplished by considering each neighboring pair of scans in turn, incrementing the starting scan by one location. Different actions can be performed depending on the attributes of the neighboring pair of scans. If both neighboring scans are within the y-range, then each scan can accumulate a weighting of half the time difference between the scans. Alternatively, if both scans are outside the y-range, then each scan can accumulate a weighting of half the pixel's time range time (1−f1+f2). In this case, f1 is the fraction of the total inter-scan time difference for which the scan exceeds the pixel's time range, and f2 is the same quantity for the other scan. This weighting can serve to accumulate the fraction of the total integrated abundance over time between these scans which intersects the smaller temporal region of the pixel. As another alternative, if one scan (e.g. “a”) is inside the pixel's y-range but the other scan (e.g. “b”) is outside the y-range, then the time overlap between the time interval of the pixel (e.g. “R”) and the time interval between the scans (e.g. “S”) can be determined. Then a weighting of R[1−R/(2S)] for scan “a” can be accumulated and a weighting of R2/(2S) for scan “b” can be accumulated. After these weightings have been accumulated for each scan, the total abundance within the pixel can be tallied as the sum over each scan of Aᵢ times the total weight for that scan.

Fifth, individual pixel values can be accumulated into a single “image” of size W×H. The image can be provided as an output comprising a pixel representation of the data present in the data file.

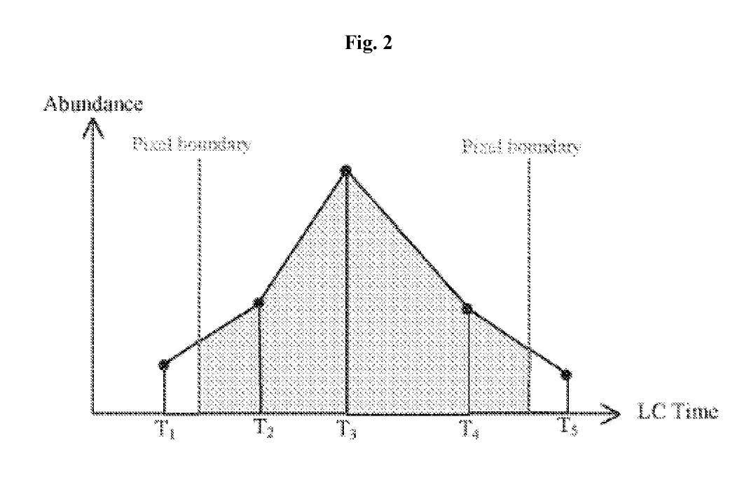

Referring to FIG. 2, an example of LC time abundance integration is shown. LC time points T1 through T5 are presented. The y-axis represents abundance values. The x-axis represents LC time values in order of increasing time from T1 through T5. Each dot represents an abundance value following integration over an mz window for a given pixel at a particular time point. For example, the dots represent abundance values following integration over mz window for each of five pixels. The shaded area represents the integrated abundance values between the pixel boundaries as shown.

LC time integration was performed using linear interpolation between these points and integrating the shaded area bordered by the pixel boundary in LC time. T₁ through T₅ were the LC times of 5 scans relevant to the computation. The pixel path was scored by identifying the edges of the pixels and including abundance between pixel boundaries indicated by the shaded area as part of peptide abundance. The area outside of the peptide boundaries was not scored as part of the peptide abundance.

Identifying features from the sample injection can comprise identifying peaks in the image file generated using the raw data for the sample injection. Identifying peaks in the image file can comprise performing a peak detection process using the image file (e.g., peak picker). Peaks can be identified by applying the peak detection process to data in the image file. Peaks identified by the peak detection process may comprise features corresponding to monoisotopic eluting peptides. The peak detection process can include identifying an mz value and an LC time value for each peak. In some cases, mass spectrometry measurements used to generate the raw data can include one or more of mass spectrometry, tandem mass spectrometry measurements, and liquid chromatography-mass spectrometry. For example, a detection process can be applied to determine LCMS features from an image file generated using raw data for a sample subjected to liquid chromatography-mass spectrometry (LCMS) measurements.

A peak detection process can include receiving an image file generated based on raw data collected for a sample injection subjected to mass spectrometry measurements. The peak detection process can include receiving as an input a data file containing mass spectrometry data (e.g., MS1 data, a “apims1” file). The input data file can comprise an image file. An output can be generated comprising locations (e.g., mz values, LC time values) of peaks. In some cases, the output can comprise peak height values and peak area values. For example, the peak detection process can include identifying mz values, LC time values, peak height values, and peak area values of peaks corresponding to monoisotopic features.

The peak detection process can utilize one or more constants. The peak detection process can use a first constant for peak detection threshold (e.g. “PEAK_DETECTION_THRESHOLD”). The first constant can be set to equal to 100, although other numbers are consistent with the operation of various embodiments of the process. In some embodiments, the first constant can be set equal to at least 10, 20, 30, 40, 50, 60, 70, 80, 90, 100, 110, 120, 130, 140, 150, 160, 170, 180, 190, 200, or 250. In some embodiments, the first constant can be set equal to no more than 10, 20, 30, 40, 50, 60, 70, 80, 90, 100, 110, 120, 130, 140, 150, 160, 170, 180, 190, 200, or 250. The peak detection process can use a second constant for delta time in seconds (e.g. “DELTA_TIME_SEC”). The second constant can be set equal to 0.5, although other numbers are consistent with the operation of various embodiments of the process. In some embodiments, the second constant can be set equal to at least 0.1, 0.2, 0.3, 0.4, 0.5, 0.6, 0.7, 0.8, 0.9, or 1.0. In some embodiments, the second constant can be set equal to no more than 0.1, 0.2, 0.3, 0.4, 0.5, 0.6, 0.7, 0.8, 0.9, or 1.0. The peak detection process can use a third constant for kernel mz width (e.g. “KERNEL_MZ_WIDTH”). The third constant can be set equal to 0.1, although other numbers are consistent with the operation of various embodiments of the process. In some embodiments, the third constant can be set equal to at least 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19 or 0.20. In some embodiments, the third constant can be set equal to no more than 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19 or 0.20. The peak detection process can use a fourth constant for delta mz (e.g. “DELTA_MZ”). The fourth constant can be set according to region as determined below. The process can use a fifth constant for kernel time width (e.g. “KERNEL_TIME_SEC_WIDTH”). The fifth constant can be set equal to 2.5, although other numbers are consistent with the operation of various embodiments of the process. For example, the fifth constant can be set equal to at least 0.5, 1.0, 1.5, 2.0, 2.5, 3.0, 3.5, 4.0, 4.5, or 5.0. The fifth constant can be set equal to no more than 0.5, 1.0, 1.5, 2.0, 2.5, 3.0, 3.5, 4.0, 4.5, or 5.0. The peak detection process can use a sixth variable for mz integration width (e.g. “MZ_INTEGRATION_WIDTH”). The sixth constant can be set equal to 0.15, although other numbers are consistent with the operation of various embodiments of the process. For example, the sixth constant can be set equal to at least 0.05, 0.1, 0.15, 0.2, 0.25, 0.3, 0.35, 0.4, 0.45, or 0.5. The sixth constant can be set equal to no more than 0.05, 0.1, 0.15, 0.2, 0.25, 0.3, 0.35, 0.4, 0.45, or 0.5. The peak detection process can use a seventh constant for time integration width (e.g. “TIME_SEC_INTEGRATION_WIDTH”). The seventh constant can be set equal to 5, although other numbers are consistent with the operation of various embodiments of the process. For example, the seventh constant can be set equal to at least 1, 5, 10, 15, 20, 25, 30, 35, 40, 45, or 50. The seventh constant can be set equal to no more than 1, 5, 10, 15, 20, 25, 30, 35, 40, 45, or 50.

An example of the peak detection process work flow is as follows. First, the mass spectrometry data (e.g., MS1 data) can be provided. For example, the mass spectrometry data can be provided as a series of rasters, such as a series of four rasters. The series of rasters can be generated using one or more rasterization processes described herein. A series of rasters can be provided whose time spacing can be DELTA_TIME_SEC and whose m/z spacing can be a function of m/z such that the part-per-million m/z spacing stays constant or substantially constant. Examples of spacings (in m/z units) for this work flow are provided in the Table 1.

TABLE 1

| Raster | | High | |
|---|---|---|---|
| number | Low m/z | m/z | DELTA_MZ |
| 1 | 0 | 500 | 0.0003 |
| 2 | 500 | 1000 | 0.0005 |
| 3 | 1000 | 2000 | 0.001 |
| 4 | 2000 | MAX | 0.002 |

Each raster can be treated separately for the purposes of detecting the peaks. The data for each raster can be provided as R(i,j), where i and j are array indexes into the m/z and LC data dimensions, respectively.

Second, a 2-dimensional Gaussian kernel can be generated. The Gaussian kernel may be generated for the purpose of convolving with the mass spectrometry data (e.g., MS1 image data) to facilitate the peak detection. This kernel may be created as the product of two 1-dimensional Gaussians with one along the m/z axis and the other along the LC axis. Each Gaussian kernel can be a sampled Gaussian function with interval DELTA_MZ or DELTA_TIME_SEC (depending on the axis), and has standard deviation KERNEL_MZ_WIDTH/2 or KERNEL_TIME_SEC_WIDTH/2 (depending on the axis). The Gaussian function may be sampled symmetrically around its peak, with the number of samples being the lowest odd integer sufficient to encompass 3 standard deviations of the kernel. Each of these sampled kernels can be normalized to sum to one. The final kernel, then, may be represented by:

$$K ( i , j ) = N exp [ - \frac{1}{2} {( \frac{i - w / 2}{{σ}_{mz}} )}^{2} - \frac{1}{2} {( \frac{j - h / 2}{{σ}_{LC}} )}^{2} ] ,$$

where N is a normalization factor, i is the zero-based MZ index into the array, j is the LC time index into the array, w is the width of the kernel in pixels, h is the height of the kernel in pixels, and σ_{mz} and σ_{LC} are the standard deviations of the kernel in sample units across the m/z and LC axes, respectively.

Third, a standard 2-dimensional convolution operation can be performed between the raster R(i,j) and the kernel K(i,j). Since the kernel is normalized to sum to unity, this convolution can preserve the total aggregate pixel abundance in the image R (with the exception of the image border regions, on the scale of the kernel's extent). This convolution operation can reduce the pixel-by-pixel noise in the raster to enable the detection of features as local maxima in the raster. The resulting raster of this convolution is C(i,j).

Fourth, each location in C(i,j) may be examined to (1) determine if its value is not less than PEAK_DETECTION_THRESHOLD and to (2) determine if its value is larger than each of the other values in its 8 nearest neighbors. Locations where these two conditions are satisfied can be local maxima of the convolution whose values are above the peak detection threshold. These local maxima can correspond to features. The mz and LC time coordinates of these features can be determined by direct transformation from the pixel coordinates (i,j) to the (mz, LC) plane.

Fifth, the peak height of a given feature may be given by the value of the convolved image C(i,j) at the location of the identified peak. The peak area can be the average of the un-convolved image across a rectangular region of pixels, and therefore can relate to the total abundance across some part of the elution. The rectangle for averaging pixels can be centered on each feature and can cover an mz width of MZ_INTEGRATION_WIDTH and an LC width of TIME_SEC_INTEGRATION_WIDTH. These widths can be adjusted to cover or approximately cover the width of a single peak (e.g., about 0.15 m/z units, though this can vary across m/z and can be about 0.05, 0.10, 0.11, 0.12, 0.13, 0.14, 0.15, 0.16, 0.17, 0.18, 0.19, 0.20, or 0.25 m/z units) and the elution time of a feature (about 5 seconds for the UHPLC pumps). The widths can be large enough to cover more than a small part of the peak, thus making them less likely to give rise to false abundance changes due to chromatographic shape change. The widths can be small enough so as not to include one or more of other peaks and low-abundance noise. The widths may be not too small to give rise to false abundance changes and not too large to include other peaks or low-abundance noise. The current values can be approximations, such as best educated guess choices.

Some embodiments comprise methods of automated mass spectrometry analysis and computer systems configured to perform MS1 feature isotopic filtering and deconvolution (e.g. using peptide isotopic models). Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

Provided herein are processes for determining isotopic cluster monoisotopic (A0) peak locations and charge states from the total set of detected peaks. The total set of detected peaks can be provided, for example, using a peak detection process as described herein. Isotopic clusters of features can be identified using a feature isotopic filtering and deconvolution process. A subset of the peaks identified using one or more peak detection processes described herein can be selected using the feature isotopic filtering and deconvolution process.

An isotopic filtering and deconvolution process can include receiving as an input peak data generated using one or more peak detection processes described herein. In some cases, the peak data can be stored in a tab-delimited format (e.g. “.mzt” file) and/or as serialized java objects. Each peak can comprise one or more of a corresponding m/z value, retention time location (e.g., LC time value), abundance, and chromatographic properties (e.g. peak width). The isotopic filtering and deconvolution process can output a subset of the total set of input peaks identified by a peak detection process, where the subset of peaks can comprise A0 peaks of molecular feature isotopic clusters. In some cases, standard operation mode can include writing these feature peaks to a database in a molecular features table. In some cases, formatted text output (.mzt) can also be specified.

An isotopic filtering and deconvolution process can utilize one or more constants during its execution. An isotopic filtering and deconvolution process can use a first constant for contrast threshold (e.g. “CONTRAST_THRESHOLD”). The first constant can be set equal to 50, although other numbers are consistent with the operation of various embodiments of the process. For example, the first constant can be set equal to at least 10, 20, 30, 40, 50, 60, 70, 80, 90, or 100. The first constant can be set equal to no more than 10, 20, 30, 40, 50, 60, 70, 80, 90, or 100. The isotopic filtering and deconvolution process can use a second constant for low mass calibrant mz (e.g. “LOW_MASS_CALIBRANT_MZ”). The second constant can be set equal to 299.2945, although other numbers are consistent with the operation of various embodiments of the process. The isotopic filtering and deconvolution process can use a third constant for high mass calibrant mz (e.g. “HIGH_MASS_CALIBRANT_MZ”). The third constant can be set equal to 1221.9906, although other numbers are consistent with the operation of various embodiments of the process. The isotopic filtering and deconvolution process can use a fourth constant for delta mz da matrix (e.g. “DELTA_MZ_DA_MATRIX”). The fourth constant can be set equal to 0.0015, although other numbers are consistent with the operation of various embodiments of the process. The isotopic filtering and deconvolution process can use a fifth constant for delta LC time matrix (e.g. “DELTA_LCTIME_SEC_MATRIX”). The fifth constant can be set equal to 0.5, although other numbers are consistent with the operation of various embodiments of the process. For example, the fifth constant can be set equal to at least 0.1, 0.2, 0.3, 0.4, 0.5, 0.6, 0.7, 0.8, 0.9, or 1.0. The fifth constant can be set equal to no more than 0.1, 0.2, 0.3, 0.4, 0.5, 0.6, 0.7, 0.8, 0.9, or 1.0. The isotopic filtering and deconvolution process can a sixth constant for mz region window (e.g. “MZ_REGION_WINDOW_DA”). The sixth constant can be set equal to 5, although other numbers are consistent with the operation of various embodiments of the process. For example, the sixth constant can be set equal to at least 1, 2, 3, 4, 5, 6, 7, 8, 9, or 10. The sixth constant can be set equal to no more than 1, 2, 3, 4, 5, 6, 7, 8, 9, or 10. The isotopic filtering and deconvolution process can use a seventh constant for LC region window (e.g. “LC_REGION_WINDOW_SEC”). The seventh constant can be set equal to 6, although other numbers are consistent with the operation of various embodiments of the process. For example, the seventh constant can be set equal to at least 1, 2, 3, 4, 5, 6, 7, 8, 9, or 10. The seventh constant can be set equal to no more than 1, 2, 3, 4, 5, 6, 7, 8, 9, or 10. The isotopic filtering and deconvolution process can use an eighth constant for mz ppm tol (e.g. “MZ_PPM_TOL”). The eighth constant can be set equal to [20+5*(n−1)].

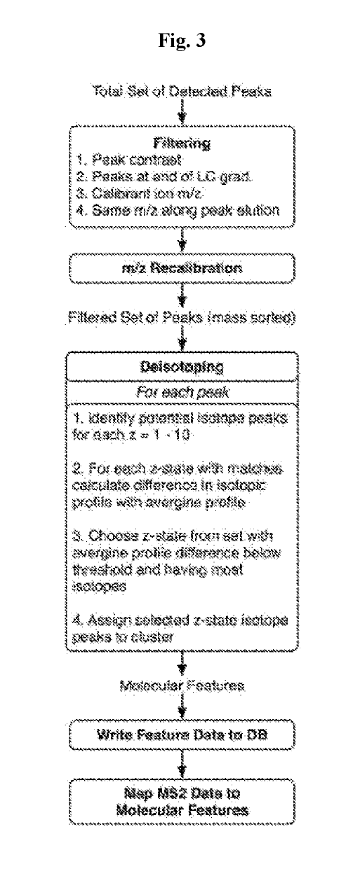

Referring to FIG. 3, an example of an isotopic filtering and deconvolution process work flow is provided. The isotopic filtering and deconvolution process can include receiving as an input a set of detected peaks. A first filtering process can be performed using a total set of detected peaks. The first filtering step can include peak contrast filtering to filter out peaks detected from the background noise, detected peaks at the end of the LC gradient (push region), m/z locations of known calibrant analytes, and spurious peaks detected along the elution profile of a given feature. Next, m/z recalibration can be performed using low and high lock mass m/z values. After the filtered set of peaks is mass sorted, each peak can be subjected to a number of processing steps during deisotoping. These deisotoping processing steps can include examining each region peak, wherein charge state matches from z=1 to 10 are tested against the current peak starting with n=1 for the isotope number to identify a set of potential isotope peaks for each of the investigated z states that produced matches. Next, if z-state isotope matches for the current peak were found, the pattern of isotopic heights for each z state can be compared against a peptide avergine isotopic model based upon neutral mass of the potential feature to calculate the difference in isotopic profile. The average of these differences across all of the identified isotopes can be calculated to provide a score for each z state indicating how well the observed isotopic profile fits the model peptide avergine profile. The z-state for the feature can then be assigned the z-state with an avergine profile difference below a threshold avergine score and having the most isotope peaks. The selected z-state isotope peaks can be assigned to the identified isotopic cluster. These molecular feature isotopic clusters can then be extracted and written to a database. For injections with MS2 scans, these scans can be mapped to identified molecular features.

As described herein, first, an isotopic filtering and deconvolution process can include providing a set of input peaks, such as a total set of input peaks identified using one or more peak detection processes described herein. Second, a peak contrast filtering can be performed to filter out background noise. Peak contrast filtering can be performed for one or more peaks of the input peaks. For example, peak contrast filtering can be performed for each peak of the input peaks provided. Contrast filtering for an input peak can comprise performing a calculation step using the following: peak_height−max(base_line_height_before_peak, base_line_height_after_peak). Peak_height can be a height of a detected peak. base_line_height_before_peak, and base_line_height_after_peak can be heights at the end of the feature's chromatographic profile before and after the peak, respectively. The max function can be used to find the higher of these two base line heights to calculate the contrast. This contrast can represent the height of the peak above the surrounding background along the chromatographic axis. Peaks with contrast values less than or equal to CONTRAST_THRESHOLD can be excluded from continued processing. For example, features corresponding to peaks with contrast values less than a contrast threshold can be disregarded from further analysis.

Third, a second filtering step can be performed to remove detected peaks at one or more of the end of the LC gradient (e.g., push region), m/z locations of known calibrant analytes, and spurious peaks detected along the elution profile of a given feature. Features with LC times greater than [0.95*total LC time] can be excluded from continued processing. Features with m/z values of {1521.96, 1221.99, 1222.99, 922.0, 622.0} can be removed from continued processing. Features within 5 ppm and within the elution profile time of a given can be removed, for example to exclude detected features that are detectable when a small mass shift occurs during the elution of a feature.

Fourth, after filtering has been performed, the m/z values of all of the features can be recalibrated using low and high lock mass m/z values LOW_MASS_CALIBRANT_MZ and HIGH_MASS_CALIBRANT_MZ. From the remaining set of non-filtered peaks, peaks with m/z values within 25 ppm of LOW_MASS_CALIBRANT_MZ and HIGH_MASS_CALIBRANT_MZ can be found, and the mean low mass and high mass m/z values can be calculated. The slope and intercept of an m/z correction line can then be calculated from the mean low and high mass values from the data, and the expected low and high mass values LOW_MASS_CALIBRANT_MZ and HIGH_MASS_CALIBRANT_MZ. The slope can be calculated according to the following formula: slope=((HIGH_MASS_CALIBRANT_MZ−meanHighMZ)−(LOW_MASS_CALIBRANT_MZ−meanLowMZ))/(meanHighMZ meanLowMZ). The intercept can be calculated according to the following formula: intercept=(LOW_MASS_CALIBRANT_MZ−meanLowMZ)−slope*meanLowMZ. The m/z values of the peaks can then be corrected based upon the following parameters: mz cal=mz+intercept+slope*mz, where mz is the original m/z value of the feature, and intercept and slope are the calibration line parameters defined above.

Fifth, a 2D matrix can be initialized and used to bin the peaks along the m/z and LC time axes using bin widths of DELTA_MZ_DA_MATRIX and DELTA_LCTIME_SEC_MATRIX. This matrix can be used to quickly look up nearby peaks within specified m/z and LC time regions during the isotopic clustering step.

Sixth, using the binned peaks, the peaks can be combined into isotopic clusters (e.g., A0, A1, A2, . . . peaks) by searching for peaks at higher masses having values with m/z=n/z, where n is the isotopic peak number, and z=1-10 (e.g., matches for all charge states in this range are considered in the search).

From the total list of m/z sorted peaks, all peaks within MZ_REGION_WINDOW_DA and LC_REGION_WINDOW_SEC of the current peak can be selected for consideration in isotopic cluster membership (e.g., region peaks). Each region peak can be examined, wherein charge state matches from z=1 to 10 can be tested against the current peak starting with n=1 for the isotope number. If the region peak is within MZ_PPM_TOL of the expected n/z value, and the peak is within LC_REGION_WINDOW_SEC, and the ratio of heights between the current peak and the region peak is less than HEIGHT RATIO TOL, then this peak can be added to the isotopic cluster list for the current peak for that z. When an isotope match is found, n is incremented to search for higher order isotopes. This process can produce a set of potential isotope peaks for each of the investigated z states that produced matches. If no matches are found for any z state, the next peak in the total list can be considered and the process restarts at the step where all peaks within MZ_REGION_WINDOW_DA and LC_REGION_WINDOW_SEC of the current peak are selected for consideration in isotopic cluster membership (region peaks).

Next, if z-state isotope matches for the current peak were found, the pattern of isotopic heights for each z state can be compared against a peptide avergine isotopic model based upon neutral mass of the potential feature. For each isotopic peak, a normalized height can be calculated by dividing by the height of the A0 peak. The difference between this height and a similarly normalized height from the avergine model can be calculated. The average of these differences across all of the identified isotopes can be calculated. This provides a score for each z state indicating how well the observed isotopic profile fits the model peptide avergine profile.

The z-state for the feature can then be assigned the z-state that has the most number of isotope peaks with an avergine score below 0.4. An identifier, such as an ID (e.g., a unique ID), can be assigned to all peaks in the identified isotopic cluster. These peaks can also be excluded from further processing.

Seventh, after all peaks from the total list have been processed, the monoisotopic peaks can be extracted and written to a database (CLIENT_DATA). The m/z, LC time, peak_height and area, and chromatographic information about these peaks can be stored in the database.

Eighth, for injections with MS2 scans (e.g., tandem mass spectrometry scans), these scans may be mapped to identified molecular features by looking for m/z and LC time matches to the molecular features. Since the instrument can trigger MS2's on non-A0 peaks, the mapping procedure can look for matches to isotopic peaks in addition to the monoisotopic peaks. For each MS2 scan, the m/z and LC time of the scan can be compared again each peak in each isotopic cluster. Scans outside of the m/z and LC time ranges of the entire isotopic clusters may be immediately rejected for matching. For scans nearby a given isotopic cluster, the closest isotopic peak for the cluster along m/z can be found. If the mass difference in ppm is less than SCAN_PEAK_MATCH_PPM, and the scan is within the LC profile of this closest cluster peak, then the scan can be assigned to the molecular feature of the matching cluster.

Provided herein are one or more processes for selecting peptides to target for mass spectrometry based sequencing such as, for example, in tandem mass spectrometry or MS/MS (e.g., MS2-based sequencing). In tandem mass spectrometry, peptides can be ionized and separated by mz (mass-to-charge ratio) in a first analyzer (MS1). Peptides from the first analyzer can then be selected for fragmentation and analysis by a second analyzer to carry out MS2-based sequencing. Peptides separated by the first analyzer may vary in the probability of successful MS2-based sequencing. One or more MS1 based metrics can be used to evaluate likelihood of successful sequencing to facilitate prioritizing of peptide selection for MS2-based sequencing.

Provided herein are one or more processes to select peptides for sequencing. A peptide selection process can be used to determine one or more quality control metrics which can correlate with successful mass spectrometry-based analysis. The peptide selection process can determine MS1-based metrics that tend to correlate with the probability of successful MS2-based sequencing. A peptide selection process can comprise receiving as an input mass spectrometry data, such as mass spectrometry data of a first analyzer (e.g., MS1 spectrum information). The input can comprise MS1 spectrum of an isotopic envelope of a feature and its estimated mz and charge state. The input often comprises MS1 spectrum information for a group of peptides that are then analyzed using the peptide selection process. The output can be metrics which correlate with the likelihood of successful sequencing. The successful sequencing can be peptide sequencing carried out by a second analyzer during tandem mass spectrometry analysis of a sample.

The peptide selection process can use one or more constants. The peptide selection process can use a first constant for low preceding offset (e.g. “LOW_PRECEDING_OFFSET”). The first constant can be set equal to 2, although other numbers are consistent with the operation of various embodiments of the process. For example, the first constant can be set equal to at least 1, 2, 3, 4, 5, 6, 7, 8, 9, or 10. The first constant can be set equal to no more than 2, 3, 4, 5, 6, 7, 8, 9, or 10. The peptide selection process can use a second constant for high preceding offset (e.g. “HIGH_PRECEDING_OFFSET”). The second constant can be set equal to 0.5, although other numbers are consistent with the operation of various embodiments of the process. For example, the second constant can be set equal to at least 0.1, 0.2, 0.3, 0.4, 0.5, 0.6, 0.7, 0.8, 0.9, or 1.0. The second constant can be set equal to no more than 0.1, 0.2, 0.3, 0.4, 0.5, 0.6, 0.7, 0.8, 0.9, or 1.0.

An example of a peptide selection process work flow is as follows. First, an mz value can be set as the m/z of the selected feature, and h can be set as the MS1 scan value at this m/z. hp can be set to be equal the maximum MS1 scan value in the interval [mz-LOW_PRECEDING_OFFSET, mz-HIGH_PRECEDING_OFFSET]. The max preceding ratio can be set equal to hp/11, although other numbers are consistent with the operation of various embodiments of the process. For example, the max preceding ratio can be optionally set equal to at least hp/2, hp/3, hp/4, hp/5, hp/6, hp/7, hp/8, hp/9, hp/10, hp/11, hp/12, hp/13, hp/14, hp/15, hp/16, hp/17, hp/18, hp/19, or hp/20. The max preceding ratio can be optionally set equal to no more than hp/2, hp/3, hp/4, hp/5, hp/6, hp/7, hp/8, hp/9, hp/10, hp/11, hp/12, hp/13, hp/14, hp/15, hp/16, hp/17, hp/18, hp/19, or hp/20.

Second, hw can be set equal to the MS1 scan value at m/z=mz+1/(2*z), where z is the charge of the species. The hw value can represent the height of the MS1 scan at the midpoint between the monoisotopic and first isotopic peaks in the envelope of the selected feature. The well ratio can be set equal to hw/h.

Provided herein are one or more processes for carrying out mass defect analysis. Mass defect analysis can be employed to assess the chemical relationship of molecular features observed in mass spectra, such as, for example, the number of nitrogen atoms in in a given class of compounds or the number of monomeric units in a molecular polymer. An extension of this analysis, described herein, can be used to provide a probability metric that a given observable molecular mass is derived from a specific class of biomolecules. The nominal mass of a molecule can be defined as the sum of the integer masses of the most abundant isotopes of the constituent atoms in the molecule. For example, a N₂ molecule would have a nominal mass of 28 atomic mass units since the most abundant nitrogen atom isotope has a nominal mass of 14 atomic mass units. In contrast, the exact mass of a molecule is the sum of the non-integer masses of the most abundant isotopes of the constituent atoms in the molecule. As an example, the exact mass of a N₂ molecule would have an exact mass of 28.03130. The difference between the nominal mass and the exact mass of a molecule can be referred to as the mass defect. In relation to mass spectrometry and the analysis of accurately measured masses, mass defect can be the offset in fractional mass a given mass value is from the nearest integer mass. A positive mass defect describes an observed mass value with a fractional mass defined by a range such as, for example, 0.0 to 0.49. A negative mass defect describes values with a fractional mass defined by a range such as, for example, 0.50 to 0.99. For example, following this rule the exact monoisotopic molecular weight for oxygen is characterized as having a negative mass defect, while that of nitrogen as having a positive mass defect. A positive mass defect can optionally describe an observed mass value with a fractional mass defined by a range from 0.0 to 0.9, from 0.0 to 1.9, from 0.0 to 2.9, from 0.0 to 3.9, from 0.0 to 4.9, from 0.0 to 5.9, from 0.0 to 6.9, 0.0 to 7.9, or 0.0 to 8.9. A negative mass defect can optionally describe values with a fractional mass defined by a range from 0.10 to 0.99, 0.20 to 0.99, 0.30 to 0.99, 0.40 to 0.99, 0.50 to 0.99, 0.60 to 0.99, 0.70 to 0.99, 0.80 to 0.99, or 0.90 to 0.99.

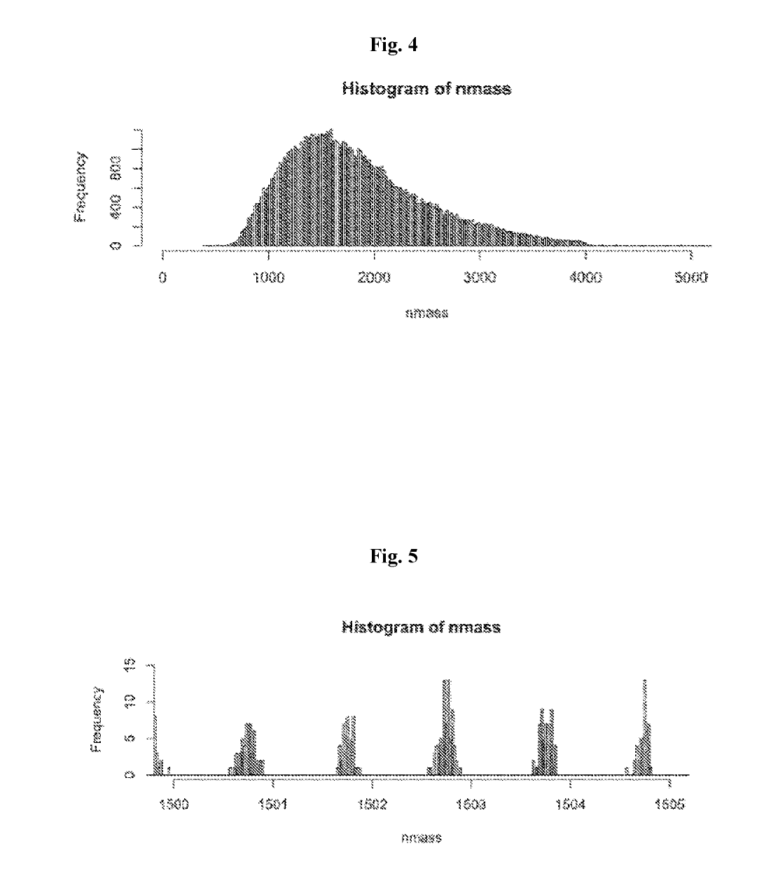

FIG. 4 shows a distribution of neutral mass molecular weights from known human peptides (approximately 86,000 peptides), with a median peptide molecular weight of about 1500 Daltons. FIG. 5 is an expanded view of the peptide molecular weight histogram showing discrete populations for each nominal mass (integer mass). As seen in FIG. 5 there can be a limited fractional mass range for peptides of a given molecular weight. Moreover, a normal distribution for each nominal mass is evident. By assuming that a normal distribution can be used to describe the population of peptides for a given nominal molecular weight, a mass defect probability can be used to describe the confidence that an observed accurate mass is that of a particular peptide.

A mass defect analysis process can comprise receiving an input comprising a library of exact neutral mass values for a list of chemicals or molecules. The library is often an extensive library of known chemical or bi-chemical exact neutral mass values. However, any given library of exact masses can be used to generate a mass defect probability histogram. As an example, a library can be a library of known petroleum organic molecules, biologically derived lipids, phospholipids, peptides, carbohydrates, nucleic acids, other molecules, or any combination thereof. A library can comprise the exact mass values for predicted peptides created by protein digestion. The library can comprise the exact mass values for predicted peptides generated by one or more specific digestion enzymes (e.g. trypsin). For example, a digestion enzyme can be trypsin, chymotrypsin, LysC, LysN, AspN, GluC, ArgC, or other protease. Each protease can leave a distinct pattern of predicted peptides due to differences in cleavage sites, and thus a sample would need to be matched with a corresponding library of exact mass values for predicted peptides based on the digestion enzyme used.

Peptides can be chosen as the targeted class of biomolecules, although other targeted classes of molecules are also contemplated. For example, the mass defect analysis described herein can be performed for other macromolecules such as lipids, carbohydrates, and nucleic acids. In some embodiments, small molecules, polymers, synthetic compounds, and/or other analytes may be analyzed using one or more mass defect analysis processes described herein.

A mass defect probability can be used to describe the confidence that an observed accurate mass is that of a particular peptide, for example due in part to an assumption that a normal distribution can be used to describe the population of peptides for a given nominal molecular weight. A library of exact masses can be based on predicted peptides such as those expected from trypsin digested proteins. Other proteases such as chymotrypsin, LysC, LysN, AspN, GluC, ArgC, or any combination thereof may be used as the basis for generating a library of exact masses. The exact neutral mass values of the predicted peptides can be provided as input for calculating the histogram of mass defect. The output can be a table of paired values (e.g. “EXACT_MASS”). When peptides are chosen as the targeted class of biomolecules, a number of constant variables can be used during the data analysis. Since peptides comprise amino acids, a library can comprise peptide exact mass values for the amino acids, such as for every amino acid. The library of amino acids can vary depending on the species from which the sample was obtained. For example, non-standard amino acids include selenocysteine and pyrrolysine. The mass defect analysis process can use one or more constants from the library to perform data analysis. Examples of constants with known exact mass values corresponding to amino acids and other constituent molecules or atoms (e.g., as indicated by the variable names) is illustrated in Table 2.

TABLE 2

| Constants | Exact Mass Value |
|---|---|
| PROTON_EXACT_MASS | 1.00727646688 |
| HYDROGEN_EXACT_MASS_DA | 1.0078250321 |
| OXYGEN_EXACT_MASS_DA | 15.99491463 |
| NITROGEN_EXACT_MASS_DA | 14.0030740052 |
| ALANINE_EXACT_MASS_DA | 71.0371137878 |
| ARGININE_EXACT_MASS_DA | 156.1011110281 |
| ASPARAGINE_EXACT_MASS_DA | 114.0429274472 |
| ASPARTIC ACID_EXACT_MASS_DA | 115.026943032 |
| CYSTEINE_EXACT_MASS_DA | 103.0091844778 |
| GLUTAMIC ACID_EXACT_MASS_DA | 129.0425930962 |
| GLUTAMINE_EXACT_MASS_DA | 128.0585775114 |
| GLYCINE_EXACT_MASS_DA | 57.0214637236 |
| HISTIDINE_EXACT_MASS_DA | 137.0589118624 |
| ISOLEUCINE_EXACT_MASS_DA | 113.0840639804 |
| LEUCINE_EXACT_MASS_DA | 113.0840639804 |
| LYSINE_EXACT_MASS_DA | 128.0949630177 |
| METHIONINE_EXACT_MASS_DA | 131.0404846062 |
| PHENYLALANINE_EXACT_MASS_DA | 147.0684139162 |
| PROLINE_EXACT_MASS_DA | 97.052763852 |
| SERINE_EXACT_MASS_DA | 87.0320284099 |
| THREONINE_EXACT_MASS_DA | 101.0476784741 |
| TRYPTOPHAN_EXACT_MASS_DA | 186.0793129535 |
| TYROSINE_EXACT_MASS_DA | 163.0633285383 |
| VALINE_EXACT_MASS_DA | 99.0684139162 |

An example of a mass defect analysis process workflow is as follows. First, a library of exact mass peptide values can be provided. For example, the library can be read into a memory (e.g. a memory located on a computing device or a server). Second, each discrete population of exact mass values can be normalized.

Some embodiments comprise methods of automated mass spectrometry analysis and computer systems configured to evaluate likelihood a given mass spectrometry spectrum, such as a MS1 spectrum, derives from a peptide rather than another molecular species. For example, a peptide confidence assessment process can be performed to obtain MS1p metric. The metric can be indicative of the likelihood that a given MS1 spectrum derives from a peptide instead of another molecular species. Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

A peptide confidence assessment process can comprise receiving an input comprising a mz value (e.g. ACCURATE_MZ), a z value (e.g. ACCURATE_Z), and peptide ion probabilities calculated from the density histogram of peptide exact mass values determined for all predicted peptide ions from a given protein database (e.g. EXACT_MASS_PROBABILITY_VALUES), or any combination thereof. An output can comprise a metric value (e.g. MS1p). The metric value can be within a range indicative of a confidence level. For example, the metric value can be closer or at a high end of the range to indicate high confidence that a spectrum derives from a peptide (e.g., high peptide confidence), or be closer or at the end of the range to indicate low confidence that a spectrum derives from a peptide (e.g., low peptide confidence). In some cases, the metric can vary from 0 to 1, with 0 representing low peptide confidence and 1 representing high peptide confidence. It is understood that other ranges can be consistent with the operation of various embodiments of the peptide confidence assessment process described herein.

The peptide confidence assessment process can use one or more constants. For example, the peptide confidence assessment process can use a constant proton mass constant (e.g. “PROTON_EXACT_MASS_DA”). The constant can be set equal to 1.00727646688, which is the mass of a proton quantified in atomic mass units or Daltons.

A peptide confidence assessment process can provide a metric value (e.g. MS1p). The process can comprise assessing the masses of all peaks from a fragmentation spectrum to arise at a single number indicating how well these masses match expected masses for peptide fragment y and b ions. An example of a peptide confidence assessment process work flow is as follows. First, a library of exact mass peptide values can be provided. For example, a library of exact mass peptide values can be read into memory as an Object EXACT_MASS_PROBABILITY VALUES. Second, ACCURATE_NEUTRAL_MASS can be determined, such as according to the formula: ACCURATE_NEUTRAL_MASS=(ACCURATE_MZ*ACCURATE_Z)—(PROTON_EXACT_MASS_DA*ACCURATE_Z). Third, DEFECT_PROBABILITY can be determined, such as by interpolation of the EXACT_MASS_PROBABILITY_VALUES using the ACCURATE_NEUTRAL_MASS.

Some embodiments comprise methods of automated mass spectrometry analysis and computer systems configured to evaluate likelihood that a mass spectrometry spectrum derives from a peptide, rather than another molecular species. For example, a peptide confidence assessment process can be performed to obtain an MS2p metric. The metric can indicate the likelihood that a given MS2 spectrum derives from a particular species instead of another species. Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

A peptide confidence assessment process can comprise assessing the masses of all peaks from a fragmentation spectrum to arise at a single number indicating how well these masses match expected masses for peptides. A peptide confidence assessment process can comprise receiving an input comprising an MS2 spectrum (e.g., tandem mass spectrometry spectrum). The MS2 spectrum can comprise pairs of mz and abundance for each spectral peak. An output can comprise a metric value (e.g. MS2p). The metric value can be within a range indicative of a confidence level. For example, the metric value can be closer or at a high end of the range to indicate high confidence that a spectrum derives from a peptide (e.g., high peptide confidence), or be closer or at the end of the range to indicate low confidence that a spectrum derives from a peptide (e.g., low peptide confidence). In some cases, the metric can vary from 0 to 1, with 0 representing low peptide confidence and 1 representing high peptide confidence. It is understood that other ranges can be consistent with the operation of various embodiments of the peptide confidence assessment process described herein.

An example of a peptide confidence assessment process work flow is as follows. First, a ms1p value p_i for peak of N peaks for each peak in the MS2 spectrum can be calculated. Second, the abundance of peak i can be defined to be A_i. The MS2p result can be set equal to Σᵢ₌₁^{N}Aᵢpᵢ/Σᵢ₌₁^{N}Aᵢ. Ms2p can be the weighted average of the ms1p values for all peaks, with each peak weighted by its abundance in the spectrum.

Some embodiments comprise methods of automated mass spectrometry analysis and computer systems configured to perform QC peak clustering and identification. Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

Provided herein are one or more processes for gauging mass spectrometry instrument performance. Mass spectrometry instrument performance can be gauged through assessment of a set of molecular features (MF) using observed intrinsic properties. A standard set of molecular features can be identified by observed intrinsic properties. For example, intrinsic properties can include observed mass/charge (MZ), chromatography position (LC), or any combination thereof, and can be important for collecting statistics on the difference between observed and expected values.

An input can be a list of targeted molecular features with attributes such as, for example, EXACT_MASS, CHARGE_STATE, and ELUTION_TIME_SEC. An output can include accurate neutral mass, charge state, observed chromatographic elution time, or any combination thereof for each molecular feature in the list. An output can also include average accurate mass offset, average observed chromatographic elution time offset, or any combination thereof for each list of molecular features.

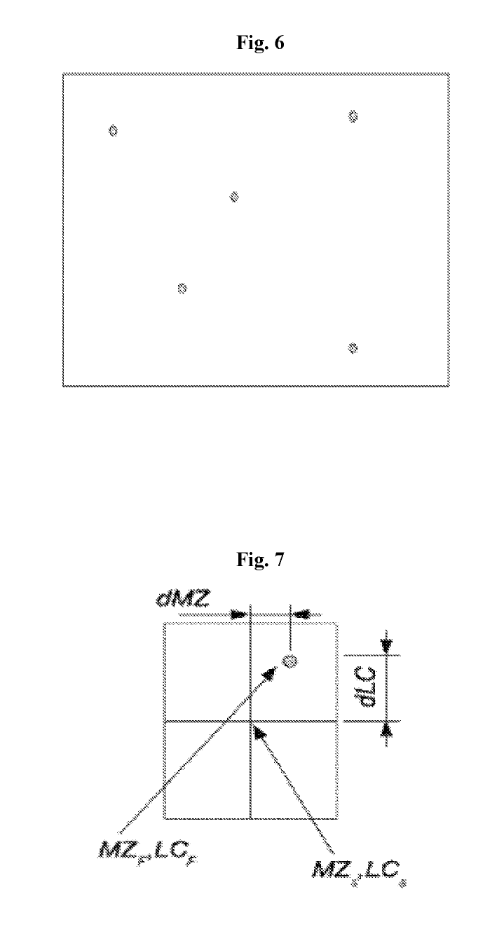

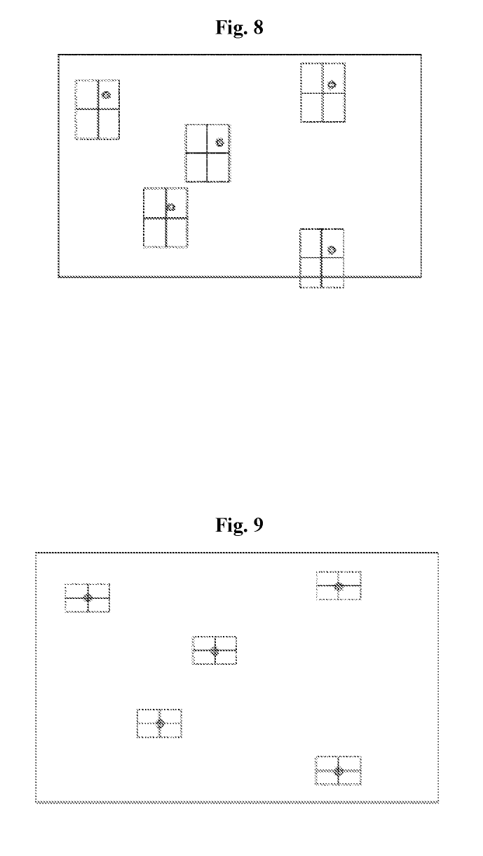

A standard set or list of molecular features can be located. Locating a standard set of molecular features (FIG. 6) may use a restricted search space defined by center Cₛ=(MZₛ, LCₛ) with a given height H and width W, for which the delta between Cₛ and the C_{f} can be calculated (FIG. 7). FIG. 6 illustrates a set of molecular features indicated by the dots spread throughout the figure. Each feature can have a particular position (MZ, LC). FIG. 7 illustrates a restricted search space defined by a center G=(MZₛ, LCₛ) with a given height H and width W. The restricted search space can encompass a molecular feature defined by a center C_{f}=(MZ_{f}, LC_{f}). The change in quantity or delta between G and the C_{f} can be calculated. FIG. 8 illustrates the application of a restricted search space to a population of molecular features. The delta between the Cₛ and the C_{f} can be calculated for each molecular feature in the population. Next, the average delta between Cₛ and C_{f} for the population of features can be used to define a shift in LC and MZ position, for which the search can be applied again with restricted size until all features can be realized with limited to no additional ride alongs. FIG. 9 shows the restricted search spaces and their position relative to the molecular features after one or more iterations of this process of shifting the LZ and MZ positions based on the average delta between Cₛ and C_{f} (FIG. 9). As shown in FIG. 9, following one or more iterations of adjustment or shifting, each of the five restricted search spaces can be centered on a corresponding molecular feature. In some cases, each restricted search space can be centered on a single corresponding molecular feature without any additional features not intended to be captured within the search space.

A mass spectrometry tool assessment process can utilize one or more constants. The mass spectrometry tool assessment process can use a first constant for maximum delta time (e.g. “DELTA_TIME_MAX_SEC”). The first constant can be set equal to 180, although other numbers are consistent with the operation of various embodiments of the process. For example, the first constant can be set equal to at least 30, 40, 50, 60, 70, 80, 90, 100, 110, 120, 130, 140, 150, 160, 170, 180, 190, 200, 250, 300, 350, 400, 450, or 500. The first variable can be set equal to no more than 30, 40, 50, 60, 70, 80, 90, 100, 110, 120, 130, 140, 150, 160, 170, 180, 190, 200, 250, 300, 350, 400, 450, or 500. The process can use a second constant for minimum delta time (e.g. “DELTA_TIME_MIN_SEC”). The second constant can be set equal to 12, although other numbers are consistent with the operation of various embodiments of the process. For example, the second constant can be set equal to at least 1, 2, 3, 4, 5, 6, 7, 8, 9, 10, 11, 12, 15, 20, 25, 30, 35, 40, 45 or 50. The second variable can be set equal to no more than 1, 2, 3, 4, 5, 6, 7, 8, 9, 10, 11, 12, 15, 20, 25, 30, 35, 40, 45 or 50. The process can use a third constant for delta mz max ppm (e.g. “DELTA_MZ_MAX_PPM”). The third constant can be set equal to 30, although other numbers are consistent with the operation of various embodiments of the process. For example, the third constant can be set equal to at least 10, 20, 30, 40, 50, 60, 70, 80, 90, or 100. The third constant can be set equal to no more than 10, 20, 30, 40, 50, 60, 70, 80, 90, or 100. The process can use a fourth constant for delta mz min ppm (e.g. “DELTA_MZ_MIN_PPM”). The fourth constant can be set equal to 10, although other numbers are consistent with the operation of various embodiments of the process. For example, the fourth constant can be set equal to at least 1, 5, 10, 20, 30, 40, 50, 60, 70, 80, or 90. The fourth constant can be set equal to no more than 1, 5, 10, 20, 30, 40, 50, 60, 70, 80, or 90. The process can use a fifth variable for time offset (e.g. “OFFSET_TIME_SEC”). The fifth constant can be set equal to 0, although other numbers are consistent with the operation of various embodiments of the process. For example, the fifth constant can be set equal to at least 1, 2, 3, 4, 5, 6, 7, 8, 9, or 10. The fifth constant can be set equal to no more than 1, 2, 3, 4, 5, 6, 7, 8, 9, or 10. The process can use a sixth constant for mz ppm offset (e.g. “OFFSET_MZ_PPM”). The sixth constant can be set equal to 0, although other numbers are consistent with the operation of various embodiments of the process. For example, the sixth constant can be set equal to at least 1, 2, 3, 4, 5, 6, 7, 8, 9, or 10. The sixth constant can be set equal to no more than 1, 2, 3, 4, 5, 6, 7, 8, 9, or 10. The process can use a seventh constant (e.g. “REJECT_IF_Z_DIFF”). The seventh constant can be set equal to FALSE. The process can use an eighth constant (e.g. “REJECT_MULTIPLE_FEATURES”). The eighth constant can be set equal to FALSE. The process can use a ninth constant (e.g. “MULTIPLE_FEATURE_SORT”). The ninth constant can be set equal to ABUNDANCE_DESC.

An example of a mass spectrometry tool assessment process can work flow is as follows. First, a list of targeted molecular features can be provided. For example, a list of targeted molecular features can be provided as Object TARGET_POPULATION. Second, a list of molecular features can be provided. For example, a list of molecular features can be provided as Object ROOT_POPULATION.

Third, for each element in the ROOT_POPULATION, DELTA_TIME_SEC and DELTA_MZ_PPM can be calculated. If the sum of DELTA_TIME_SEC and OFFSET_TIME_SEC is less than DELTA_TIME_MAX_SEC, and the sum of DELTA_MZ_PPM and OFFSET_MZ_PPM is less than DELTA_MZ_MAX_PPM, the element from the ROOT_POPULATION can be added to an array of key-value pairs CLUSTER_POPULATION.

Fourth, the resultant CLUSTER_POPULATION can be sorted for each TARGET_POPULATION element by MULTIPLE_FEATURE_SORT. If REJECT_MULTIPLE_FEATURES is FALSE, then the each element in the CLUSTER_POLULATION with multiple features can be discarded. However, if REJECT_MULTIPLE_FEATURES is not FALSE, then each non-top result for each element in the CLUSTER_POLULATION with multiple features can be discarded.

Fifth, the AVERAGE_DELTA_TIME_SEC for the resultant CLUSTER_POPULATION can be calculated. Sixth, AVERAGE_DELTA_MZ_PPM for the resultant CLUSTER_POPULATION can be calculated. Seventh, OFFSET_TIME_SEC can be set equal to AVERAGE_DELTA_TIME_SEC. Eighth, OFFSET_MZ_PPM can be set equal to AVERAGE_DELTA_MZ_PPM. Ninth, DELTA_TIME_MAX_SEC can be set equal to max(DELTA_TIME_MIN_SEC, (0.5*DELTA_TIME_MAX_SEC)). Tenth, DELTA_MZ_MAX_PPM can be set equal to max(DELTA_MZ_MIN_PPM, (0.5*DELTA_MZ_MAX_PPM)).

Eleventh, the CLUSTER_POPULATION can then be evaluated. Evaluating the CLUSTER_POPULATION can comprise determining if DELTA_MZ_MAX_PPM is equal to DELTA_MZ_MIN_PPM) and DELTA_TIME_MAX_SEC is equal to DELTA_TIME_MIN_SEC. If DELTA_MZ_MAX_PPM is equal to DELTA_MZ_MIN_PPM) and DELTA_TIME_MAX_SEC is equal to DELTA_TIME_MIN_SEC CLUSTER_POPULATION can be returned as an output. Otherwise, if the preceding condition is not satisfied, then steps one through eleven can be repeated.

Some embodiments comprise methods of automated mass spectrometry analysis and computer systems configured to assess digestion, oxidation, alkylation, or any combination thereof. Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

One or more methods described herein can comprise a process for evaluating one or more inaccuracies attributable to defects included in the sample being analyzed. A sample defect evaluation process can include quantifying one or more of an extent of unintended chemical modifications and amount of undigested proteins present in a sample injection. Chemical modifications can include laboratory induced chemical modifications such as, for example, one or more of oxidation and alkylation. For example, chemical modification caused by a mass spectrometry tool can be evaluated and the quantity of undigested protein can be determined to reduce or eliminate inaccuracies. Digestions of proteins can be performed using one or more of various types of proteases such as trypsin, chemotrypsin, ArgC, AspN, GluC, LysC, pepsin, thermolysin, or any combination thereof. Evaluating these chemical modifications and/or digestions can advantageously facilitate assessing the quality of instrument platform performance such as, for example, mass spectrometry instrumentation, LCMS, MALDI-TOF, or other instrument platforms used to identify biomolecules.

A sample defect evaluation process can comprise receiving an input comprising molecular features tagged with peptide sequences and post-translational modifications determined for tandem mass spectra via the open mass spectrometry search algorithm (OMSSA), given a calculated False Discovery Rate. The output can include values that represent a ratio of chemical modification given the total number of assigned tandem mass spectra.

An example of a sample defect evaluation process is as follows. First, a list of search engine results tagged to targeted molecular features can be provided. For example, a list of search engine results tagged to targeted molecular features can be provided as Object PEPTIDE_POPULATION. Second, for each element in the PEPTIDE_POPULATION, the number of molecular features tagged with a given post-translational modification can be counted and the number of molecular features tagged with a peptide containing an internal K (alanine) or R (arginine) can be counted. For example, a (POST_TRANS_MOD_COUNT) and a (TRYP_MISS_CLEVAGE_COUNT) can be returned. Third, a percentage of molecular features tagged with a given post-translational modification can be provided. For example, a POST_TRANS_MOD_COUNT/PEPTIDE_POPULATION can be returned. Fourth, the percentage of molecular features tagged with a peptide containing an internal K (alanine) or R (arginine) can be provided. For example, a TRYP_MISS_CLEVAGE_COUNT/PEPTIDE_POPULATION can be returned.

Some embodiments comprise methods of automated mass spectrometry analysis and computer systems configured to perform quality control (QC) analysis using various metrics. Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

QC analysis can be configured to evaluate instrument platform performance. The platform is often a mass spectrometry tool, including LCMS, MALDI-TOF, or any other instrument platforms used to identify biomolecules. The QC analysis can be performed regularly such as before each sample injection, or on an hourly, daily, weekly, biweekly, monthly, biannual, annual basis, or biennial basis. In some cases, QC analysis may be performed daily, such as prior to initiating sample data collection. In some cases, QC analysis can be performed at predetermined intervals each day, such as to determine whether sample data collection should continue. QC analysis can reduce or minimize the collection of bad data and/or reduced or prevent wasting valuable clinical samples due to instrumentation problems. One or more instrument QC testing procedures provided herein can improve or ensure that tools, including LCMS instruments, are meeting one or more predetermined performance metrics prior to running and/or continuing to run sample injections. The one or more performance metrics can be configured to evaluate instrument performance for one or more of mz values, retention time values and feature abundances. For example, a QC analysis may be configured to determine whether an LCMS instrument performs within specified tolerances along one or more of three main axes of LC/MS data: m/z, retention time, and feature abundance. One or more of the QC analyses described herein evaluate the instrument performance on these three aspects of the data. One or more of such QC analyses processes can be performed prior to, between, and/or after, running sample injections. The analyses results can be used to decide whether sample data collection should proceed and/or continue.

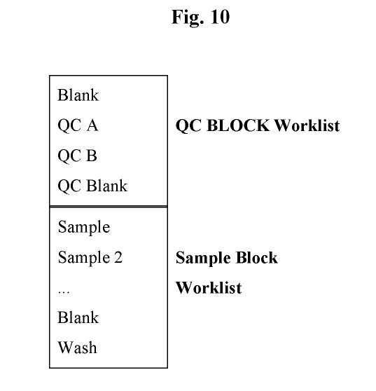

Referring to FIG. 10, an example of a QC analysis workflow is shown. A QC block worklist can be performed prior to a sample block worklist. A sample block worklist can be preceded by a QC block worklist, where the sample block worklist proceeds if a passing QC score on instrument functional performance is obtained in the QC block worklist. Data collection can be performed on a worklist basis, where a worklist can comprise a block of injections. A worklist can comprise a sequence of injections, for example a series of LC/MS injections. Referring to FIG. 10, a QC worklist can comprise a block of injections comprising a blank injection (“Blank”), a first QC injection (“QC A”), and a second QC injection (“QC B”), followed by a QC blank injection (“QC Blank”). A composition used for QC can comprise a background plasma matrix containing manually added peptides of known m/z, retention time, and concentration values. These peptides produce known LC/MS signals and can therefore be used to assess one or more of three main functional performances of the mass spectrometry instrument: mass accuracy, LC reproducibility (e.g. retention time, peak shape), and abundance measurement accuracy (e.g. abundance consistency, known ratios). In some cases, data collection for sample injections may not proceed until a passing score for each of mass accuracy, LC reproducibility and abundance accuracy is obtained.

In some cases, each QC composition can contain 12 added peptides, 6 of which have differing concentrations between the QC A injection and the QC B injection. The differing concentrations of the 6 peptides can be used to evaluate the ability of the instrument to detect known abundance changes.

Eight QC assessment metrics can be used to evaluate the three functional performances of the mass spectrometry tool, to thereby enable generation of LC/MS data of desired quality: (1) number of detected peptides, (2) relative change in the number of molecular features compared to control data, (3) maximum abundance error across the peptides relative to control values, (4) overall mean abundance shift from all peptides compared to the control value abundances, (5) standard deviation of the abundance ratio errors between QC A and QC B, (6) maximum peptide m/z deviation relative to control values, (7) maximum peptide retention time deviation relative to control values, and (8) maximum peptide chromatographic full-width half max (FWHM). A QC analysis process can use fewer than the eight metrics. For example, depending on the functional performances of interest to a user, one or more of these metrics may be chosen in any combination to address one or more of the three functional performances for quality LC/MS data as part of the QC assessment. Data collection for sample injections can proceed if all selected metrics demonstrate a passing score. For example, a QC assessment optionally evaluates at least 1, 2, 3, 4, 5, 6, 7, or 8 of the metrics. As another example, a QC assessment can optionally evaluate no more than 1, 2, 3, 4, 5, 6, or 7 of the metrics.

In some cases, all eight metrics can be analyzed for a QC test such that a mass spectrometry tool passes the QC test if all eight metrics of the tool are within predetermined corresponding tolerance limits (e.g., control values). The predetermined tolerance limits may be calculated as described in further details herein. Failure of the mass spectrometry tool to demonstrate metrics within the predetermined tolerance values can prevent the execution of the sample block worklist, for example enabling instrument issues to be identified and/or resolved prior to sample injection. Predetermined tolerance values can be determined from a defined set of QC injections which are deemed of passing quality by expert consensus. These predetermined tolerance values can be stored for reference in one or more of a database of the mass spectrometry tool, on the file system of the mass spectrometry tool, and a database of an associated computing device.

Provided herein is an example of a peptide selection and tool evaluation process for QC analysis. First, peptides of known mass, retention time, and concentration are selected for the QC test. These peptides can be added to the QC A and B injections in order to produce LC/MS signals for assessment. One set of peptides, the reconstitution (RC) peptides, can be placed in the protein reconstitution mixture, and are therefore present in both QC injections and sample injections. A second set, the spike-in (SI) peptides, can be added only to QC injections, and in differing amounts between the QC A injection and QC B injection. The SI peptides can be used to assess the ability of the instrument to detect peptide abundance changes. The following Table 3 summarizes the properties of examples of these QC peptides, including columns for peptide designations, peptide sequences, m/z values, retention times in seconds (RT values), and QC A:B concentration ratios for each QC peptide:

TABLE 3

| | | | | QC A:B |
|---|---|---|---|---|
| | | | | Concen- |
| Peptide | | | RT | tration |
| designation | Sequence | m/z | (sec) | Ratio |
| RC.1 | LGGGGGGDGSR | 445.2097 | 276 | 1.00 |
| RC.2 | LGGGGGGDFR | 446.7172 | 672 | 1.00 |
| RC.3 | LLGGGGGDFR | 474.7485 | 1086 | 1.00 |
| RC.4 | LLLGGDFR | 445.7583 | 1746 | 1.00 |
| RC.5 | LLLLDFR | 445.2789 | 2448 | 1.00 |
| RC.6 | LLLLLDFR | 501.8209 | 2874 | 1.00 |
| S1.1 | PEKTAAPASDPTG | 621.3040 | 486 | 0.33 |
| S1.2 | RVAQIRTEIENSD | 510.9338 | 894 | 3.00 |
| Si.3 | EISTNIRQAGVQYSR | 574.6358 | 1068 | 3.00 |
| S1.4 | RINSAKDDAAGLQIA | 514.9459 | 1164 | 0.33 |
| SI.5 | SYVPSAEQI | 497.2480 | 1362 | 0.33 |
| SI.6 | AGGGVTLLQAAPALD | 677.3722 | 2034 | 3.00 |

The following QC metrics can be used to assess instrument performance based upon the data acquired from the QC A and B injections.

As a first metric, a minimum number of detected QC peptides from QC A and QC B injections can be determined. For example, the minimum number of peptides detected in the QC A and QC B injections can be determined according to the following formula:

N_{peptides}^{min}=min(N_{peptides}^{A},N_{peptides}^{B})

The set of peptides used in evaluating the first metric may be specified so observation of the specified peptides is needed to achieve a passing score for the first metric. For example, a passing score for this metric comprises observation of a predetermined set of 9 peptides, instead of just observation of any 9 peptides.

As a second metric, a change in the number of molecular features for a QC type can be determined. For example, the change can be determined according to the following formula:

$${err}_{mf} = \frac{{n}_{mf}^{QCtype}}{mean ( {n}_{mf}^{ctrl , QCtype} )} - 1$$

This value can represent the signed change in the count of molecular features for a given QC injection, as compared to an average number of features computed from the control data. This metric can provide information indicative of one or more of carry over, contamination (e.g., observed relative increase in features), and a loss in instrument sensitivity (e.g., observed relative decrease in features).

As a third metric, abundance relative to a control abundance for each peptide, i, by QC type, can be determined. To determine abundance relative to a control abundance, a an abundance correction and/or normalization via a geometric mean and a relative error in peptide abundance can be calculated. An abundance correction and/or normalization via a geometric mean can be determined, for example according to the following formula:

$$\text{?} = \frac{{abn}_{i}}{\text{?} ( {abn}_{i} )}$$

$$\text{?} \text{indicates text missing or illegible when filed}$$

A relative error in peptide abundance may be calculated, for example according to the following formula:

$${dev}_{i} = \vert \frac{{abn}_{i}^{corr} - {abn}_{i}^{ctrl , QCtype}}{{abn}_{i}^{ctrl , QCtype}} \vert$$

The abundance value, abn, can be the integrated abundance across m/z and RT for the monoisotopic peak of each peptide. For each QC injection, the peptide abundances can be normalized by the geometric mean abundances across all peptides for that injection, for example equivalent to a linear shift in logarithmic abundance space, which can be a method used during quantitation. These normalized values can then be compared against the fitted control values, as described in further details here. The abundance deviations (devᵢ) can represent the fractional change in abundance compared to the expected, fitted abundance. Several QC metrics can be obtained from resulting distribution of the deviations (e.g. mean, max absolute deviations).

As a fourth metric, abundance shift of a given QC sample relative to control abundances overall all peptides, for each QC type, can be calculated, for example according to the following formula:

log 2abn_{shift}^{QCtype}=meanᵢ(log₂(abn_{QCtype,i}))−log₂(abnₘ^{ctri,QCtype})

Abundance shift expressed as a percent change may be calculated according to the following formula:

abn_{shift}^{QCtype}=2^{log2abn}^{_{shift}}^{^{QCtype}}−1

In this case, the mean, un-normalized log 2 peptide abundances for a given QC injection are compared to the corresponding quantity from the control data, where the control abundances for the individual peptides are the mean log 2 abundances across the control datasets. This metric can be used to assess overall changes in instrument sensitivity.

As a fifth metric, abundance ratio between QC A and QC B for each peptide, i, can be calculated, for example according to the following formula, which provides log 2 ratio correction factor for QC A and B:

$$log 2 {ratio}_{mean} = {mean}_{i} ( {log}_{2} ( \frac{{abn}_{QCA , i} / {abn}_{QCB , i}}{{ratio}_{i}} ) )$$

The correction factor for the ratios can be calculated according to the following formula:

$${ratio}^{carr} = {2}^{- log 2 {ratio}_{mean}}$$

$${err}_{i}^{ratio} = \frac{{abn}_{QCA , i} / {abn}_{QCB , i}}{{ratio}_{i} / {ratio}^{carr}} - 1$$

The parameters of this distribution can be used to assess the performance of detected abundance differences.

As a sixth metric, mass accuracy (e.g. in ppm) compared to the average historical control values for each peptide, i, can be calculated, for example according to the following formula:

$${err}_{i}^{ppm} = {10}^{6} \vert \frac{{mz}_{i} - {mz}_{i}^{ctrl}}{{mz}_{i}^{ctrl}} \vert$$

As a seventh metric, retention time deviation from an average historical control values for each peptide, i, by QC type, can be calculated, for example according to the following formula:

$${RT}_{i}^{ctrl , QCtype} = \frac{1}{{N}_{QCtype}} {\sum }_{\text{?}} {RT}_{d , i}$$

$$\text{?} \text{indicates text missing or illegible when filed}$$

An eighth metric can be a peak shape, for example comprising a full-width half max (FWHM) value along the chromatography axis for each peptide, i.

QC metric control values can be used as comparison points for the various metrics described herein. QC metric control values can be established using historical data. Historical data selected for establishing control values may be of known quality, such as known to be of good and/or high quality. The control values may be established prior to running QC tests. One or more sets of control values for the peptides may be calculated. At least one, two, three, four, five, six, seven, eight, nine, or ten sets of control values for the peptides can be calculated. The control values can include average m/z values, average retention time values, fitted abundance values, or any combination thereof. For example, three sets of control values for the peptides can be calculated: average m/z values, average retention time values, and fitted abundance values.

First, for m/z control values, the mean m/z over all datasets in the control data for each peptide, i, can be calculated, for example according to the following formula:

$${mz}_{i}^{ctrl} = \frac{1}{N} {\sum }_{\text{?}} {mz}_{d , i}$$

$$\text{?} \text{indicates text missing or illegible when filed}$$

This average can be computed over all datasets, regardless of QC type.

Second, for retention time control values, the mean retention time for each peptide by QC type (QC A or QC B) can be calculated according to the following formula:

$${RT}_{i}^{ctrl , QCtype} = \frac{1}{{N}_{QCtype}} {\sum }_{\text{?}} {RT}_{d , i}$$

$$\text{?} \text{indicates text missing or illegible when filed}$$

Two retention time control values can be calculated for each peptide, one for QC A and one for QC B. The average can be performed over datasets of only one QC type, for both QC types.

Third, for abundance control values, the fitted abundances for each peptide by QC type can be calculated, for example according to the following formula:

log 2(abn₂^{ctrl,QCtype})−lm(log 2(areaMona)˜peptide+dataFile,data=controlData)

The formula above represents a linear model (specified in R code) used to fit the abundances. This model determines the best fit for the logarithmic abundance of each peptide in each QC type, while allowing an independent logarithmic shift normalization for each injection. The result of the model is the expected pattern of logarithmic peptide abundances across the peptides within the QC A and B samples. A separate model is fit for each QC type.

Fourth, the mean log 2 abundances (geometric means) can be first calculated for each peptide individually across the control data by QC type according to the following formula:

log 2abnᵤₜ^{ctrlQCtype}=meanᵢ(log 2AbnMeanQC_{types})

Then the overall mean of these values for all peptides can be calculated by QC type representing the overall mean abundance level of the control data according to the following formula:

In the QC test, this overall mean abundance level can be compared to the mean of log 2 peptide abundances from the testing sample to find the relative abundance shift.

Fifth, a mean number of molecular features by QC type can be calculated as the arithmetic mean of the molecular features counts across the control data for each QC type.

The following Table 4 provides an example of a set of QC test metrics and corresponding thresholds.

TABLE 4

| QC Metric | Test Threshold |
|---|---|
| Minimum Observed Peptides | All 9 peptides observed |
| | from peptides |
| | RC.2-RC.5, S1.2-SI.6 |
| Relative change in molecular features count: | <0.30 |
| \|err_{mf}\| | |
| Maximum abundance deviation for any peptide | <0.25 |
| in QC A or devᵢ B: | |
| Maximum, overall abundance shift: \|abn_{shift}\| | <0.40 |
| Abundance ratio std. dev.: sd(err^{ratio}) | <0.10 |
| Max ppm error: max(err^{ppm}) | <10 ppm |
| Max RT error: max(err^{RT}) | <40 sec |
| Max FWHM: max(FWHM) | <32 sec |

Some embodiments comprise methods of automated mass spectrometry analysis and computer systems configured to carry out LCMS data analysis. Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

Provided herein are various processes for performing analysis of mass spectrometry data such as, for example, LCMS data analysis. Data analysis can include normalization of the mass spectrometry data, such as MS1 normalization. LCMS data analysis can be performed for sample injection analysis and/or biomarker discovery. Sample injection analysis and/or biomarker discovery can comprise comparison of peak areas across different individual samples. Peak areas as extracted from mass spectrometry data (e.g., MS1 data) can contain technical noise, some components of which may be correctable through a process of data normalization. For example, varying protein loading amounts between different samples can broadly amplify all peak areas but may not have relevance for biomarker discovery. To make the data comparable across different samples, one approach can be to multiplicatively normalize all areas to a reference value. As an example, a normalization algorithm can rely on different samples of the same type (e.g. human plasma fraction #17) containing identifiable features across samples, and that “broad” variations (e.g., as defined herein) in the abundances of those features can be employed to correct for some technical variability. Furthermore, because feature abundances may vary systematically between different instrument platforms (e.g., including upstream processing), it can be useful to derive a common value which can be compared between such platforms.

An example of a mass spectrometry data normalization process is provided herein. A set of peaks, and corresponding areas, for a set of samples can be provided. For example, an input for a normalization process can include a set of extracted de-isotoped peaks, and corresponding areas, for a set of samples of a given type. These peaks may correspond to multiple injections of the same sample type, such as injections across multiple instrument lines. These peaks may be clustered across all samples to provide an identified set of named clusters together with their corresponding features in the samples. The output can comprise a corrected peak area for each de-isotoped peak in the input set. The output produced via the data analysis can aid in biomarker discovery. The corrected peak area may be usable for statistical testing for biomarker discovery.

An example of the data normalization process can include, first, defining the set of N reference clusters as those feature clusters which correspond to exactly one feature from each sample. Second, the sample data can be divided up by instrument into sets of samples on a per-instrument basis.

Third, for each such per-instrument sample set, following can be performed. An index value s can be defined as referring to a given sample in the set (e.g., running from 1 to S for the given instrument). The log-base-10 abundance of reference cluster c's feature area in sample s can be defined as A_{cs}. a_cs can be defined as the log-abundance of the feature with the average across the sample subtracted, for example according to the following formula:

$${a}_{cs} = {A}_{cs} + \frac{1}{N} {\sum }_{{c}^{′}} {A}_{{c}^{′} s}$$

This a_cs operation can act to multiplicatively register the peak areas from different samples. Each such cluster can correspond to an m/z value mz and an aligned LC time t, which are output from the clustering algorithm for each sample. The mean log abundance value across samples can be defined as

$${μ}_{c} = \frac{1}{S} {\sum }_{S} {a}_{cs} .$$

it all samples are identical without any technical variability, then each a_{cs} would equal μ_{c}. The deviation from this ideal case can be given by δ_{cs}=α_{cs}−μ_{c}. These can act as the noise source to be modeled, for example slowly varying across mz and LC time, depending on the nature of the technical noise in the measurement system. Subtracting the mean log-area from each sample can provide zero as an average value of delta.

The noise process (delta) within each sample can be modeled as a function which is slowly-varying in both m/z and LC time. This modeling step can be accomplished by choosing a cubic equation in these two variables and fitting the parameters of the cubic within each sample. The function for a given sample s can be represented as δₛ(mz, t)=Σ_{i,j=0}³βᵢⱼ(mz)ⁱt^{j} where i and j are the polynomial powers of mz and t, respectively, βᵢⱼ is the coefficient for the corresponding term in the polynomial, and beta_00 is set to zero (since it is already corrected for in the mean subtraction). Next, in order to fit this model, the data values for delta, mz, and t can be collected for each sample and the coefficients beta can be computed using the “lm” function in R (version 2.11.1): Im (delta˜(t*mz)+1(t̂2)+l(mẑ2)+I(t̂3)+I(mẑ3)+I(t̂2*mz)+I(t*mẑ2)). The linear model can return the coefficients independently for each sample as well as a prediction function A for the deltas as a function of (mz,t) within that sample. Each log-area value a_cs can be corrected by the estimate of this function to give the corrected per-instrument log-abundance: â_{cs}=α_{cs}−Δ(mz_{c}, t_{c}).

For each feature cluster, its mean log-abundance in each instrument can be computed, using for example:

$$\overline{{α}_{c}^{i}} = \frac{1}{{S}_{i}} {\sum }_{s = 1}^{{S}_{i}} \overline{{α}_{cs}^{i}}$$

The grand mean cluster value for cluster c can be defined as the mean of this value across all instruments:

$$\overline{{μ}_{c}} = \frac{1}{{N}_{i}} {\sum }_{s = 1}^{{N}_{i}} \overline{{α}_{cs}^{i}} .$$

The corrected log-abundance for cluster c's feature in sample s which is measured on instrument i can be determined by adjusting its mean on that instrument to be the grand mean: ã_{cs}ⁱ=â_{c}ⁱ+μ̅ₛ̅.

Some embodiments comprise methods of automated mass spectrometry analysis and computer systems configured to cross sample MS1 peak clustering. Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

Methods described herein can comprise one or more processes to associate identified peaks of a common feature across samples. To facilitate comparison of data collected across different samples, identified peaks corresponding to a feature from multiple samples can be associated with the feature. One or more such processes can be applied to features identified using LCMS measurements. For example, while m/z values for a feature can typically be consistent between samples, LC time values for the feature can vary widely between samples. One or more processes described herein comprise an LC time adjustment process for adjusting LC times of features across different samples. An LC time adjustment process can be performed to adjust LC time values of common features across different samples. An LC time adjustment process can comprise clustering monoisotopic features between samples based upon the m/z and LC times of the features. In some cases, an LC time adjustment process can comprise performing non-linear retention time warping to bring feature LC times in-line across samples prior to clustering the feature across samples.

An LC time adjustment process can comprise receiving an input comprising a set of datasets to cluster (e.g., features read from a database), and clustering parameters. An output of the process can comprise a data file, such as a tsv file, comprising all identified molecular features from all of the datasets, each assigned cluster ID's based upon the cross sample RT alignment and clustering. In some cases, the output can comprise writing a retention time alignment file which provides the LC time corrections across the LC axis for each aligned dataset.

In some aspects, an LC time adjustment process can use one or more constants. The process can use a first constant CONSIDER_CHARGE_STATE. In some embodiments, CONSIDER_CHARGE_STATE can be set to true. Alternately, CONSIDER_CHARGE_STATE can be set to false. The process can use a second constant MZ_CLUSTER_WINDOW_PPM. MZ_CLUSTER_WINDOW_PPM can be set to equal 35. MZ_CLUSTER_WINDOW_PPM can be set to other values, for example to a value which is at least 1, 2, 5, 10, 15, 20, 30, 35, 50, 75, 100, 150, or more than 150. The process can use a third constant LC_CLUSTER_WINDOW_SEC. In some aspects, MZ_CLUSTER_WINDOW_PPM is no more than 1, 2, 5, 10, 15, 20, 30, 35, 50, 75, or no more than 100. LC_CLUSTER_WINDOW_SEC can be set to equal 5. In some cases, LC_CLUSTER_WINDOW_SEC can be set to another value, such as a value that is at least 1, 2, 5, 10, 15, 20, 30, 35, 50, 75, 100, 150, or more than 150. In some aspects, LC_CLUSTER_WINDOW_SEC is no more than 1, 2, 5, 10, 15, 20, 30, 35, 50, 75, or no more than 100.

An example of an LC time adjustment process workflow is provided as follows. First, from the supplied input datasets, the molecular features can be provided. For example, the molecular features can be read from a client database. Second, using a first dataset supplied in the input list of datasets as a common basis dataset, a non-linear retention time (RT) alignment for each of the other datasets can be performed against this basis. The retention times for the features can then be transformed based upon calculated alignment mapping on a dataset by dataset basis. Third, LC aligned features can be clustered across datasets using a sparse multidimensional hash map to efficiently cluster together features based upon their m/z and LC time locations. Other inputs, outputs, constants, and processes for clustering molecular features are consistent with the specification.

Some embodiments comprise methods of automated mass spectrometry analysis and computer systems configured for identifying distinct peptides across fractions of a sample. Use of the methods can comprise cross fractionation peak clustering (e.g., cross fractionation MS1 peak clustering). Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

One or more processes described herein can comprise clustering of identified peaks across fractions of a sample. The process of fractionation can be used to divide a sample into a number of separate portions, each of which containing a subset of analytes of the sample. In one example, the analytes are proteins. Peptide features of proteins in the fractions can be analyzed to generate clusters which represent distinct peptides. A cross fraction peak clustering process can be performed to group identified peaks across the fractions of a sample into clusters which represent distinct peptides included in the sample. Peptide features (e.g., accurate mass and time tags, AMTs) from a given protein can appear in different fractions of a sample, such as adjacent fractions. Peptide features which appear to be AMTs but are from fractions which are not adjacent to one another, such as fractions which are far apart from one another, can correspond to distinct peptides rather than the same peptide. A cross fraction peak clustering process can take into account the fraction in which a peptide feature lies to generate a set of clusters which nominally represent distinct peptides.

A cross fraction peak clustering process can comprise receiving an input comprising a list of features detected across the fractions of a given sample. The input may comprise one or more of a neutral mass, retention time (aligned or un-aligned), fraction number, and feature identifier for each of the detected features. The cross fraction peak clustering process can provide an output comprising a cluster designation corresponding to each detected feature. In some cases, the output can comprise associating a cluster designation with an identifier of each detected feature. These clusters can have contiguous extent across fraction number.

A cross fraction peak clustering process can use one or more constants, including a first constant MAX_DELTA_PPM. MAX_DELTA_PPM can be 30. In some cases, MAX_DELTA_PPM can have a different value, including at least 1, 2, 5, 10, 15, 20, 30, 35, 50, 75, 100, 150, or more than 150. In some aspects, MAX_DELTA_PPM no more than 1, 2, 5, 10, 15, 20, 30, 35, 50, 75, or no more than 100. The process can use a second constant MAX_DELTA_TIME_SEC. MAX_DELTA_TIME_SEC can be 10. In some cases, MAX_DELTA_TIME_SEC can have another value, including at least 1, 2, 5, 10, 15, 20, 30, 35, 50, 75, 100, 150, or more than 150. In some aspects, MAX_DELTA_TIME_SEC is no more than 1, 2, 5, 10, 15, 20, 30, 35, 50, 75, or no more than 100. The process can use a third constant MAX_CLUSTER_SIZE_PPM. MAX_CLUSTER_SIZE_PPM is often 75. In some cases, MAX_CLUSTER_SIZE_PPM can have another value, including at least 1, 2, 5, 10, 15, 20, 30, 35, 50, 75, 100, 150, or more than 150. In some embodiments, MAX_CLUSTER_SIZE_PPM is no more than 1, 2, 5, 10, 15, 20, 30, 35, 50, 75, or no more than 100. The process can use a fourth constant MAX_CLUSTER_SIZE_SEC. MAX_CLUSTER_SIZE_SEC is often 50. In some cases, MAX_CLUSTER_SIZE_SEC can have a different value, including at least 1, 2, 5, 10, 15, 20, 30, 35, 50, 75, 100, 150, or more than 150. In some embodiments, MAX_CLUSTER_SIZE_SEC is no more than 1, 2, 5, 10, 15, 20, 30, 35, 50, 75, or no more than 100.

An example of a cross fraction peak clustering process workflow is provided as follows. The process in some aspects comprises one or more steps to cluster features of identical analytes. First, a cluster can be defined to be a collection of features. The mz, time, and fraction full ranges of a given cluster can be defined as the full extent of those quantities over the contained features. Second, the process can begin with no clusters defined. Third, each neutral mass feature in turn can be compared to all existing clusters. If the feature's mz value is within MAX_DELTA_PPM ppm of the full range of a given cluster and its Ic time value is within MAX_DELTA_TIME_SEC of that cluster, and its fraction number differs by no more than one from the range of that cluster, then that feature can be determined to hit the cluster. All clusters which are hit by the feature can be merged into a single cluster. This process can be repeated over all features. If a feature does not hit any cluster, then that feature can become a lone member of a newly designated cluster.

Fourth, after each feature is clustered, each cluster can be examined for size. For example, if the feature space is too dense, there can be failures to define distinct clusters due to overlapping features. Density of feature space can be tested by ensuring that no cluster has a maximum mz PPM extent greater than MAX_CLUSTER_SIZE_PPM and a maximum LC time extent no greater than MAX_CLUSTER_SIZE_SEC. Any cluster which fails these criteria can be broken up into individual clusters, one per feature within the cluster.

Other methods comprising alternative inputs, outputs, constants, processes, or other components for clustering features by fraction are consistent with the specification.

Some embodiments comprise methods of automated mass spectrometry analysis and computer systems configured to assess the cross-fractionation fractionation performance. Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

One or more processes described herein comprise selecting a subset of fractions of a sample for analysis. A sample can be fractionated to provide a plurality of fractions of the sample. Use of fractionation in processing of samples can result in a significant amount of time used for mass spectrometry analysis (e.g., time for LCMS analysis, MALDI-QTOF, or other suitable instrument analysis platform) of all fractions from a given sample. A subset of the fractions can be selected for further analysis, such as for feature identification, such as to facilitate reduced processing time (e.g., as compared to analysis of all fractions of the sample) while enabling extraction of desired information from the sample. A fraction subset selection process described herein can comprise selecting a subset of fractions of a sample for further processing, such as for mass spectrometry measurements. Sub-selecting a number of fractions for mass spectrometry analysis can advantageously provide increased processing speed. A fraction subset selection process can be configured to select fractions so as to obtain the desired information with fewer than the total number of fractions, such as selecting fractions which have a higher probability of providing more unique pieces of information. The process can determine which fractions contain the more non-redundant pieces of information (e.g. which fractions provide the largest number of non-redundant clusters, peptides, proteins). The process can be configured to select a subset of fractions to reduce information loss from the sub-selection, such as due to forgoing analysis of non-selected fractions of the sample.

A fraction subset selection process can comprise receiving an inputs which is often a formatted text data file containing a textual identifier for a piece of information (e.g. peptide sequence, cluster identifier) and the fraction number it is identified in. The textual identifier and the fraction number can be provided in other formats. The fraction subset selection process can be configured to provide an output comprising one or more subsets of fractions of a sample. In some cases, the output comprises a subset of fractions which can provide the desired information (e.g., a best set of fractions) and a subset of fractions which would not provide the desired information (e.g., a worst set of fractions), for example for each set of n fractions to select. In some cases, the output comprises a minimum, maximum, and average counts for the information counts by n, for example contained in an output file separate from the output file providing the fraction subsets. The output can be a formatted text file, or another suitable format.

A fraction subset selection process can use one or more constants, such as N_REP. N_REP can be adjusted up or down to control execution times. In some embodiments N_REP can be set to 5,000. In some embodiments, N_REP can be set to a different value, including at least 1, 2, 5, 10, 20, 50, 100, 200, 500, 1,000, 2,000, 5,000, 10,000, 20,000, 50,000, 100,000, 1,000,000 or more than 1,000,000. In some embodiments N_REP is at most 1, 2, 5, 10, 20, 50, 100, 200, 500, 1,000, 2,000, 5,000, 10,000, 20,000, 50,000, 100,000, 1,000,000 or at most 1,000,000.

An example of a fraction subset selection process workflow is provided as follows. First, an input file can be provided. The input file can comprise information as described herein. A mapping data structure keyed by fraction number, can be populated with a set of character string values representing the information to quantify. For example, if an analyte such as peptide sequences are to be quantified, the map can contain the unique or non-redundant set of peptides for each fraction.

Second, for n=1 to the total number of available fractions, n fractions can be chosen at random from the total set of available fractions. From these fractions, the total number of unique or non-redundant pieces of information contained in the selected fractions can be counted using the data map constructed from the input data. For example, if a peptide sequence is stored in the data map, the number of unique or non-redundant peptide sequences found in the n randomly selected fractions can be counted. This process can be iterated for N_REP times for each n, to sample to space of n-fraction sets. During this iterative process, a minimum, a maximum, and average counts for each sampling rep and the n-fraction sets that give rise to the large and smallest counts can be stored.

Third, after the iterative steps are completed, the resulting data for each n can be reported. A random sampling approach can be used for the fraction subset selection process. The random sampling approach can be applied to reduce processing time. Exhaustive processing of all possible fraction sets can be computationally impractical and use significant processing time. A returned fraction subset for providing desired information can be based on the random sampling, for example rather than an exhaustive evaluation of all possible fraction set combinations.

Alternative inputs, outputs, constants, processes, and other components for determining the most unique parts of a dataset may be employed, consistent with the specification.

Some embodiments comprise methods of automated mass spectrometry analysis and computer systems configured to re-extract mass spectrometry features (e.g., MS1 features), and fill in the blanks. Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

One or more methods described herein can include a feature re-extraction process. The complex nature of data obtained from a mass spectrometry instrument analysis platform, such as MS1 LCMS data, can present challenges in obtaining highly reproducible data. Differences in detected features between different samples, including samples of the same type, can be observed in data from mass spectrometry tools, including from the same tool. Features may not be observed within samples of the same type due to one or more flaws in the process, such as one or more of feature co-elution, large LC time shifts unaccounted for by RT (retention time) alignment, mis-assigned charge states and monoisotopic peaks, and low abundance features. A feature re-extraction process can be performed to identify missing features, for example by reducing or eliminating the one or more flaws. A feature re-extraction procedure can be used to fill in missing feature observations, for example by using m/z and LC coordinates of features detected in other samples.

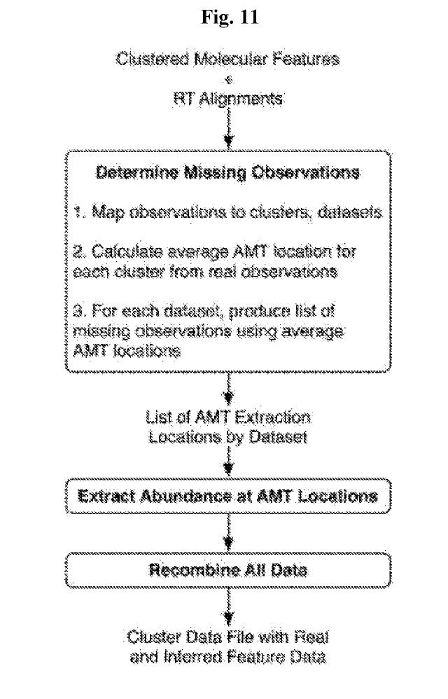

FIG. 11 is a process flow chart of an example of a feature re-extraction process.

A feature re-extraction process can comprise receiving an input comprising a clustered data file and RT alignment. The clustered data file and RT alignment can be provided as a file, for example produced by a clustering process (e.g., a cross fraction peak clustering process as described herein). The process can provide an output, such as a data file in the same format as the input clustered data file, comprising both real feature observations from the set of detected peaks, and inferred observations (e.g., fill-in's) from the feature re-extraction process. In some cases, the output file comprises additional columns to indicate other variables, such as the type of observation (e.g., real versus fill-in), and whether or not a given cluster has multiple observations from a single dataset.

An example of a cross fraction peak clustering process workflow is provided as follows. First, the input cluster data file can be provided. A hash map can be produced, which is keyed by cluster identifier (e.g., ID) found in the input cluster data file. For each cluster ID, another hash map can be stored, which can be keyed by dataset, and holds all of the molecular features found for that cluster in that dataset. The total set of datasets can be determined, such as while the file is read. The RT (retention time) alignment file can be provided to obtain the retention time mapping for each dataset.

Second, for each cluster, the real feature observations from all of the datasets they were observed in can be used to calculate the average m/z and LC time values for that cluster. The average LC time can be calculated using the RT aligned values. The most frequently occurring z-state and NMC pair can be determined for the cluster from the underlying features, which for example is needed to assign these values when cross sample MS1 peak clustering is performed without regard to charge state. Third, using the set of datasets with feature observations for a given cluster, and the total set of datasets found in the input data, the datasets missing feature observations can be determined. For these datasets, the average LC time of a given cluster can be transformed to an unaligned LC time by dataset using the RT alignment mapping. A list of these missing feature observations can be produced for each dataset. Fourth, for each dataset, an output file (e.g., in a format such as .mzt format) can be written indicating the m/z and LC time coordinate for the missing features. This file can then be used as input for feature abundance extraction in the next step.

Fifth, using the same underlying approach described in the MS1 peak detection process, inferred feature abundances can be extracted from each dataset for the missing feature locations. In this case, instead of detecting features, the feature locations can be given to the algorithm, and the feature areas can be extracted in the same way real feature observations are extracted. Sixth, after missing feature extraction has been performed, all of the extracted peak information can be collected and written out to one or more files, such as one file, in the same format as the input clusters file, but also included the inferred missing feature data.

Different inputs, outputs constants, processes, features to be analyzed, or other components may be utilized in the method to improve data reproducibility for alternative analytes or protocols, consistent with the specification.

Some embodiments comprise methods of automated mass spectrometry analysis and computer systems configured to filter features using retention times (e.g., MS/MS retention times). Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

One or more methods described herein comprise a process for filtering identified peptides using predetermined retention times. Peptides can be incorrectly identified. Search engines can select analytes, such as peptides, which are not correct assignments. Such assignments can be validated through the assessment of independent information. This independent information can comprises one or more expected values for a property, such as the expected retention time (e.g., LCMS retention time) of the peptide. The expected retention time can have predictability based on amino acid composition. A retention time filtering process can comprise constructing a filter which nulls any peptide assignment which is not consistent with a predicted retention time of the peptide. For example, a peptide identification which is not consistent with a predicted retention time is nullified.

A retention time filtering process can comprise receiving an input comprising all of the identified sequences together with their retention times from a sample injected into the MS in MS1/MS2 mode. The output, for example, can comprise a PASS/FAIL value for each such identified peptide sequence describing whether it is (PASS) or is not (FAIL) an acceptable sequence match based on retention time filtering.

A retention time filtering process can use one or more constants. In some aspects, the one or more constants comprise a first constant TRAINING_INTENSITY_P_THRESHOLD. In some embodiments, TRAINING_INTENSITY_P_THRESHOLD is 0.0001. In some cases, TRAINING_INTENSITY_P_THRESHOLD can be a different value, such as no more than 0.0001, 0.0002, 0.0005, 0.001, 0.002, 0.005, 0.01, 0.02, 0.05, or no more than 1. In some embodiments, TRAINING_INTENSITY_P_THRESHOLD is at least 0.0001, 0.0002, 0.0005, 0.001, 0.002, 0.005, 0.01, 0.02, 0.05, or more than 0.05. The process can use a second constant TRAINING_PERCENTAGE. TRAINING_PERCENTAGE often is 80%, or no more than 1%, 2%, 5%, 10%, 20%, 50%, 80%, or no more than 100%. The process can use a third constant MIN_TRAINING_SIZE. MIN_TRAINING_SIZE often is 100, or at least 1, 2, 5, 10, 20, 50, 100, 200, 500, 1,000, 2,000, 5,000, 10,000, or more than 10,000. In some embodiments, MIN_TRAINING_SIZE is no more than 1, 2, 5, 10, 20, 50, 100, 200, 500, 1,000, 2,000, 5,000, or no more than 10,000. The process can use a fourth constant MAX_TRAINING_ERROR_MIN. MAX_TRAINING_ERROR_MIN often is 7, or at least 1, 2, 5, 10, 20, 50, 100, 200, 500, 1,000, 2,000, or more than 2,000. In some aspects, MAX_TRAINING_ERROR_MIN is no more than 1, 2, 5, 10, 20, 50, 100, 200, 500, 1,000, 2,000, or no more than 2,000. The process can use a fifth constant MAX_TEST_ERROR_RATIO. In some cases, MAX_TEST_ERROR_RATIO is 1.5, or at least 1, 2, 5, 10, 20, 50, 100, 200, 500, or more than 500. In some aspects, MAX_TEST_ERROR_RATIO is no more than 1, 2, 5, 10, 20, 50, 100, 200, 500, or no more than 500. The process can use a sixth constant INTENSITY_P_THRESHOLD. In some embodiments, TRAINING_INTENSITY_P_THRESHOLD is 0.1, or no more than 0.0001, 0.0002, 0.0005, 0.001, 0.002, 0.005, 0.01, 0.02, 0.05, 0.1, 0.2, 0.5, or no more than 1. In some embodiments, TRAINING_INTENSITY_P_THRESHOLD is at least 0.0001, 0.0002, 0.0005, 0.001, 0.002, 0.005, 0.01, 0.02, 0.05, 0.1, 0.2, 0.5, or more than 0.5. The process can use a seventh constant OUTLIER_SIGMA. OUTLIER_SIGMA often is 3, or at least 1, 2, 5, 10, 20, 50, 100, or more than 100. In some aspects, OUTLIER_SIGMA is no more than 1, 2, 5, 10, 20, 50, or no more than 50.

An example of a retention time filtering process workflow is provided as follows. First, for each MS2 spectrum the MS2 intensity p-value, pl, can be computed (e.g., this can be a measure of how much more abundant are peaks which match expected peptide fragments are than those which do not match expected fragments). The lower this value, the higher the accuracy of the sequence match. Second, The training set can be defined to be a randomly chosen subset of TRAINING_PERCENTAGE of the spectra among those MS2 spectra which have pl<TRAINING_INTENSITY_P_THRESHOLD, if the size of this set is less than MIN_TRAINING_SIZE, then ABORT, giving all sequences a value PASS.

Third, for all sequences and corresponding retention times in the training set, a linear model can be solved to determine the additional retention time accrued for each amino acid in the sequence. The actual retention time can be modeled as the sum over amino acids in the sequence of the retention time coefficient assigned to that amino acid. Thus, T=Σₐ₌₁²⁰NₐTₐ where T is the peptide's retention time, a sums over the 20 amino acids, Nₐ is the count of amino acid type a in the peptide, and Tₐ is the fitted retention time model's prediction for the additional retention time afforded by the addition of a peptide of type a. This model can be solved using a function from data analysis software, such as R (version 2.11.1) using the “Im” function, resulting in a set of Tₐ values for the model. The training error can be defined to be the standard deviation of the difference between the actual and modeled retention times. If this training error is larger than MAX_TRAINING_ERROR_MIN, then all sequence matches can be passed since the model does not accurately reflect the data.

Fourth, the resulting model can be tested against the remaining (100-TRAINING_PERCENTAGE) % of the low-pl data to determine the RMS model prediction error in retention time on novel data. If the test error is larger than MAX_TEST_ERROR_RATIO times the training error, then all sequence matches can get a value of PASS (e.g., since the model does not generalize well to new data). The standard deviation of this test error can be set to σ_{T}, for example corresponding to a typical error the model produces when matching an accurate spectrum. The critical error cutoff can be defined to determine a retention time outlier σ_{C} to be OUTLIER_SIGMA times this standard deviation.

Fifth, the MS2 sequence retention time can be estimated from the model and compared to the actual retention time of the peptide. If the retention time difference is larger in magnitude than σ_{C} and the pl value for that peptide is larger than INTENSITY_P_THRESHOLD, then the peptide match can receive the value FAIL. Otherwise it can receive the value PASS.

Alternative inputs, outputs, constants, processes or other components of the method are consistent with the specification.

Some embodiments comprise methods of automated mass spectrometry analysis and computer systems configured to facilitate retention time (RT) alignment. Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

One or more methods described herein comprise a retention time alignment process. A retention time alignment process can be performed to achieve a time warping to enable improved matching of features between injections along the RT axis. A retention time alignment process can be performed in data analysis of a sample, such as to identify proteins in the sample, and/or for marker discovery. In some cases, sample analysis can comprise combining data for individual peptide features across many samples analyzed on an instrument platform, such as LCMS, MALDI-TOF, or any other instrument platform used to identify biomolecules. Each feature can have a corresponding set of coordinates given by m/z and its retention time, and these coordinates can be used in defining Accurate Mass and Time (AMT) coordinates, which can be nominally preserved across injections. LC systems can have inherent fluctuations, those retention times can experience systematic variation between injections which can be reduced or eliminated using a nonlinear time warping. For example, retention time alignment process can be configured to perform a nonlinear time warping transformation upon an LC time to correct for fluctuations of LC systems.

A retention time alignment process can comprise receiving an input comprising a list of features (e.g., MS1 features) corresponding to injections of interest, and the identification of a single injection to act as a time reference. An output of the process can comprise a functional warping of LC time in that injection onto the reference time axis for each injection specified.

A retention time alignment process can use one or more constants. The process can use a first constant NUM_TEST_POINTS. In some embodiments, NUM_TEST_POINTS is 20000.

In some cases, NUM_TEST_POINTS can be a different value, such as at least 10, 100, 200, 500, 1,000, 2,000, 5,000, 10,000, 20,000, 50,000, 100,000, 200,000, 500,000, 1,000,000, or more than 1,000,000. The process can use a second constant SECONDS_PER_WARP_SEGMENT. SECONDS_PER_WARP_SEGMENT often is 60. In some cases, SECONDS_PER_WARP_SEGMENT can be a different value, such as no more than 1, 2, 5, 10, 20, 60, 100, 200, 500, 1,000, or no more than 2,000. The process can use a third constant MAX_RT_ERROR_SEC. MAX_RT_ERROR_SEC often is one or more values for each of a number of iterations (for example 4 iterations). MAX_RT_ERROR_SEC in one example is {180,120,60,30}. In some embodiments, each value of MAX_RT_ERROR_SEC is at least 1, 2, 5, 10, 20, 50, 75, 100, 150, 200, 500, 1,000, 2,000, 5,000, or more than 5,000. The process can use a fourth constant MAX_PPM_ERROR. In some cases, MAX_PPM_ERROR is 10. In some cases, MAX_PPM_ERROR can be a different value, such as no more than 1, 2, 5, 10, 20, 50, 100, 200, 1,000, or no more than 2,000. The process can use a fifth constant POVVELL_OBJECTIVE_TOL. In some aspects, POVVELL_OBJECTIVE_TOL is 0.001. In some cases, POVVELL_OBJECTIVE_TOL can be a different value, such as no more than 0.0001, 0.0002, 0.0005, 0.001, 0.002, 0.005, 0.01, 0.02, 0.05, 0.1, 0.2, 0.5, or no more than 1.

An example of a retention time alignment process workflow is provided as follows. First a best matching feature in the reference injection corresponding to a time-warped feature F in an injection to be warped (the warp injection) can be defined as that reference injection feature whose mz differs from F's mz by no more than MAX_PPM_ERROR ppm and has the minimum retention time difference in the reference injection from the warped time in injection 1. In some cases, it is possible that no such feature exists.

Second, the time cost mismatch between a corresponding feature in two injections can be defined as Min(MAX_RT_ERROR_SEC, |t1−t2|, where t1 is the aligned RT of the feature in the first injection, and t2 is the corresponding value in the second injection. This value cannot be larger than MAX_RT_ERROR_SEC, which can be additionally used as the penalty cost for a feature which is found in only one of the injections.

Third, the total time cost mismatch between a set of N features found in injection 1 and a corresponding set of features in injection 2 can be defined as the sum over all found corresponding features of the time cost mismatch between individual features plus MAX_RT_ERROR_SEC times the number of features which cannot be identified across the injections.

Fourth, the time warping function from the warp injection to the reference injection can be defined as a function oft which takes the form of a natural cubic spline with M knots placed at regular time intervals given by Tᵢ−iΔ+τ, with i from 1 to M. For this processes time warping can be set to 0, and Delta can be SECONDS_PER_WARP_SEGMENT.

Fifth, to determine the natural cubic spline which best warps injection 1 to the reference injection, warp function can be initialized to an initial guess. Powell's method can be applied to minimize the total time cost mismatch between the two injections over the M knot values of the cubic spline. The Powell method error tolerance employed can be POWELL_OBJECTIVE_TOL. NUM_TEST_POINTS z=2,3,4 features can be chosen for matching are drawn at random from the warp injection, unless fewer are available, in which case the total number available can all used.

Sixth, to find an overall best warping function the previous step can be iterated four times, each with a different value of MAX_RT_ERROR_SEC. This can enable very large retention time offsets to be included initially while at later stages refining to smaller offsets and potentially including a different set of matching features. The resulting optimum warp from each iteration can be used as the initial warp for the next iteration.

Some embodiments comprise methods of automated mass spectrometry analysis and computer systems configured to identify the number of non-redundant proteins in a sample, including a number of minimum assignable proteins. Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

One or more methods described herein comprise a process for identifying a number of non-redundant proteins in a sample. A process for identifying a number of non-redundant proteins in a sample can comprise providing a minimum number of assignable proteins for the sample. In some cases, the number of unique analytes, such as proteins, lipids, small molecules, nucleic acids, sugars, or other biomolecules identified in a sample, can be a valuable quantifier of instrument platform performance. The platform is often LCMS, MALDI-TOF, or any other instrument platforms used to identify biomolecules. Determining a non-redundant number of proteins in a sample can be challenging, for example due to an identified analytic corresponding to multiple distinct analytes, any number of the multiple distinct analytes may actually be present in the sample. For example, an identified peptide can be from any of multiple proteins, one or more of the proteins can be present in the sample. The total analyte count, such as total protein count, can be bracketed as lying in between the maximum value of the total number of analytes which map to any analyte fragment (for example, peptides) found and the minimum value given by the lowest number of analytes, which can explain the analyte fragments identified in the sample.

A process for identifying a number of non-redundant proteins can comprise receiving an input comprising a list of identified proteins in a sample together with a mapping of each peptide to all proteins which can comprise the peptide. The process can provide an output comprising a count a minimum number of proteins which can explain the peptides found.

A process for identifying a number of non-redundant proteins can use one or more constants. The process can use a first constant MAX_TRIALS. The data analysis may comprise an iterative method to identify the proteins of interest. The number of iterations may be determined by one or more constants, for example MAX_TRIALS. MAX_TRIALS is often 12,5000. In some aspects, MAX_TRIALS is no more than 1,000, 2,000, 5,000, 10,000, 20,000, 50,000, 100,000, 200,000, 500,000, 1,000,000 or no more than 1,000,000. In some aspects, MAX_TRIALS is at least 1,000, 2,000, 5,000, 10,000, 20,000, 50,000, 100,000, 200,000, 500,000, or at least 1,000,000.

An example of a process for identifying a number of non-redundant proteins is provided as follows. First, a set of proteins which contain peptides can be divided into distinct groups of proteins which share at least one peptide with other members of the group. For example, if two proteins share a peptide, the two proteins can be members of the same protein group. In some aspects, the analysis starts with zero protein groups and the input data which maps discovered peptides to all proteins which contain them. An empty mapping can be created which will map from each protein to the protein group which contains it (e.g., protein group by protein map). For each mapping from a peptide to a set of proteins, an empty protein group can be defined (e.g., new protein group). Mapping of each peptide to a set of proteins can be iterated over each protein in the set. For example, the following can be iterated over each protein: (1) find the protein group which contains that protein, and (2) if no such group exists, add the protein to the new protein group; otherwise add all of the proteins in the group to the new protein group. For each protein in the new protein group, the value of the protein group can be set by the protein map for that protein to the new protein group, for example replacing any previous mapping.

Second, each protein group can correspond to proteins with disjoint peptides. This can split the problem into distinct sub-problems, each for a separate set of peptides whose presence in the sample needs to be explained by the fewest proteins. To determine this minimum number, the minimum number of proteins for each protein group is added up in some embodiments. In some aspects, the minimum number can be determined by accumulating into a set (e.g., peptide set) all of the peptides which were discovered and are contained in the proteins of the given protein group. In some embodiments, these are the peptides whose presence in the sample must be explained by the presence of proteins in this group. In some aspects, the protein state is defined to be a subset of proteins contained in the protein group. The protein state reflects a possible configuration of proteins present in the sample in some embodiments. In some aspects, the total number of possible protein states is determined. In some aspects, the total number of possible protein states is 2̂(number of proteins in group), that is, two to the power of the number of proteins in the group. In some cases, three proteins would thus have eight possible states. In some aspects, if this total number of protein states does not exceed MAX_TRIALS, then iteration is performed over all possible protein states. If this total number of states exceeds MAX_TRIALS, then MAX_TRIALS protein states can be chosen at random to iterate over. In some aspects, the minimum number of proteins required to cover the peptides (e.g., min count) is set to be positive infinity. In some aspects, the minimum number of proteins required to cover the peptides (e.g., min count) is set to less than positive infinity. In some embodiments, the current best protein state is set to be NULL. In some aspects, iteration over each state comprises two steps. In some embodiments, the first iterative step is to accumulate together all of the peptides for all of the proteins present in the state. In some embodiments, this represents the sample. In some aspects, the second iterative step is if this peptide accumulation equals the peptide set for the group, then this configuration of proteins covers the peptides. Alternately or in combination if this is the case, and if the number of proteins in the state is less than min count, then min count is set to this number of proteins. In some aspects, this protein state is recorded as the current best protein state. Alternately or in combination, the min count is reported as the minimum number of proteins which cover the protein group. In some aspects, the current best protein state is reported as the minimum protein state. If no such state exists (i.e. min count is positive infinity), then an error condition is reported in some embodiments. In some cases, this can occur if none of the random protein states chosen covers the peptides.

Third, the min counts for each protein group can be summed as the total minimum protein count. In some embodiments, the minimum protein states of each protein group are accumulated together in a single set as the minimum protein set. In some cases, these values are returned as the output.

Some embodiments comprise methods of automated mass spectrometry analysis and computer systems configured to provide a common search engine control interface (e.g., to provide a plug and play search engine interface). Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

One or more methods described herein comprise a process for generating a common search engine control interface. Use of multiple proteomic search engines to identify peptides to mass spectrometry data (e.g., tandem mass spectra) can be advantageous for assembling a correct and/or complete listing of observed proteins and/or peptides. Different search engines may comprise duplicate and/or overlapping information to facilitate providing a correct and/or complete listing of observed proteins and/or peptides. However, interfacing with different search engines, including third party search engines, can be difficult. For example, input and output for one third-party proteomic search engine can be different that of another. A process for generating a common search engine interface can provide consistent use of proteomic peptide assignments and annotations. Consistent use of proteomic peptide assignments and annotations can be maintained both in, and out of an automated analysis pipeline, such that the control and implementation of any third party mass spectrometry search engine is the same (e.g., tandem mass spectral search engine). In some cases, a process for generating a common search engine interface can comprise parsing an output from each engine into a conserved output form, for example enabling quick and/or common data reduction between search engine results.

A process for generating a common search engine control interface can comprise receiving an input comprising a file containing mass spectra of peptides, such as an API *.mgf file. Other input file formats can include mzML, TraML, mzIdentML, mzXML, mzData, mzQuantML, pepXML, protXML, MSF, tandem, omx, dat, FASTA, PRIDE XML, dta, MGF, ms2, pkl, PEFF, msp, splib, blib, ASF, PSI-GelML, .d, .BAF, .FID, .YEP, .WIFF, .t2d, .PKL, .RAW, .QGD, .DAT, .MS, .qgd, .spc, .SMS, .XMS, MI, .sky, .skyd, APML, or other suitable formats. The output can comprise a file containing mass spectrometry peptide assignments, such as tandem mass spectra peptide assignments. In some cases, the output can be provided in an API format *.csv file.

Constants consistent with the specification may be utilized, including constants to define error rates, ranks, expectation values, scores, the number of processing threads for analysis, the database format, the presence of additional modifications to the analytes that affect mass assignments, or additional variables involved in analyte identification. In some embodiments, constants in a process for providing a common search engine control interface can comprise PRECURSOR_ION_MAX_ERROR_PPM. In some variations of the process, PRECURSOR_ION_MAX_ERROR_PPM is 15, or no more than 1, 2, 5, 10, 20, 30, 40, 50, or 100. In some variations, PRECURSOR_ION_MAX_ERROR_PPM is at least 0.1, 0.2, 0.5, 1, 2, 5, 10, 20, 30, 40, 50, or more than 50. The process can use a second constant FRAGMENT_ION_MAX_ERROR_PPM. In some cases, FRAGMENT_ION_MAX_ERROR_PPM is 25, or no more than 1, 2, 5, 10, 20, 30, 40, 50, or 100. In some variations, FRAGMENT_ION_MAX_ERROR_PPM is at least 0.1, 0.2, 0.5, 1, 2, 5, 10, 20, 30, 40, 50, or more than 50. The process can use a third constant RANK_MIN. RANK_MIN is 1, at least 1, 2, 5, 10, 25, or more than 25 in some embodiments of the algorithm. The process can use a fourth constant EXPECTATION_VALUE_MAX. The constant EXPECTATION_VALUE_MAX is often 1, or at least 1, 2, 5, 10, 20, 30, or more than 30. Alternately, EXPECTATION_VALUE_MAX is no more than 1, 2, 5, 10, 20, or 30. The process can use a fifth constant SCORE MIN. SCORE MIN is 0, or at least 1, 2, 5, 10, or more than 25 in some embodiments of the algorithm. SCORE MIN can be no more than 1, 2, 5, 10, or 25 in other examples. The process can use a sixth constant PROCESSING_THREADS_MAX. PROCESSING_THREADS_MAX is ALL_AVAILABLE, or any number less than all available, depending on how many threads are available. The process can use a seventh constant FASTA_DATABASE. A number of different databases are used to identify analytes in a specific format, as defined by a constant variable. For example if the analyte is a protein, the variable FASTA_DATABASE is a protein-containing database such as uniprot_sprot_fasta. The process can use an eighth constant POST_TRANSLATIONAL_MODS. POST_TRANSLATIONAL_MODS can be used to indicate modifications that affect the mass of the identified protein, such as oxidation, acetyl, carbamylation, carbamidomethyl, carboxymethylation, Gln to pyro-Glu, or any other known or unknown post-translational modifications. Additional values of these variables applying to other types of data and analytes and consistent with the specification can also be used.

An example workflow for a process for generating a common search engine control interface is provided as follows. First, command line arguments can be constructed given the constants detailed above and an input file, for example *mgf, in a format specific for the given SEARCH ENGINE. Second, the execution of the SEARCH ENGINE is initiated. Third, the format specific for the given SEARCH ENGINE output file can be read and parsed into memory into an array of key-value pairs. Fourth, using a database project file, such as a MySQL Object, the array of key-values pair attributes can be inserted into the corresponding database, such as Pipeline MySQL Database given API_EXPERIMENT_NO as the primary_key.

In various embodiments of the process, SEARCH ENGINE can comprise one or more of DIA-Umpire, PRIDE, CSF-PR, Mascot, Param-Medic, TopPIC, MS2PIP, MSPathfinder, pTOp, DRIP, PIPI, MS-GF+, HiXCorr, MALDIquant, LuciPHOr, Cascaded search, WEAK, rTANDEM, shinyTANDEM, MS Amanda, MassIVE, pCluster, MS-Align+, MSPLIT, MS-GFDB, Gutentag, X! Tandem, Morpheus Search Algorithm, X! Hunter, MyriMatch, Pepitome, Tremelo, Andromeda, Crux, MS Data Miner, SearchGUI, SpectraST, MetaMorpheus, SimTandem, PeptideART, MSPrepSearch, PepFrag, pBuild, pFind, SEQUEST, Multitag, Cycloquest, or any number of other databases that allow the identification of analytes from signals, such as proteins from mass spectrometry peptide signals.

Consistent with the specification, other databases and database outputs may be used with the algorithm.

Some embodiments comprise methods of automated mass spectrometry analysis and computer systems configured to extract mass spectrometry measurements (e.g., tandem mass spectra) into a universal file. Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

One or more methods described herein comprise a process for extracting mass spectrometry measurements (e.g., tandem mass spectra) into a universal file. A mass spectrometry measurements extraction process can comprise concatenating third-party data files into universal files. An extraction process can comprise concatenating third party extracted centroid tandem mass spectra extracted into a universal file, such as a Mascot Generic File (*.mgf), or any other acceptable file format such as mzML, TraML, mzIdentML, mzXML, mzData, mzQuantML, pepXML, protXML, MSF, tandem, omx, dat, FASTA, PRIDE XML, dta, ms2, pkl, PEFF, msp, splib, blib, ASF, PSI-GelML, or other suitable formats. The process can comprise providing an output file comprising annotations of individual tandem mass spectra headers with specific attribute information.

A process for extracting mass spectrometry data can comprise receiving as an input a third party input file. For example, the third party input file can comprise a .dat tandem mass spectra attribute file. The input file may comprise other formats, including .d, .BAF, .FID, .YEP, .WIFF, .t2d, .PKL, .RAW, .QGD, .DAT, .MS, .qgd, .spc, .SMS, .XMS, MI, .sky, .skyd, APML, or any other acceptable third party input file containing data.

An example of a workflow of a mass spectrometry data extraction process is provided as follows. First, one or more files containing data such as features to be extracted from the file, for example, a file named SpecFeatures.1.tsv can be provided. For example, such files can be read into a memory. Second, the file contents can be parsed into an array of key-value(s) pairs that represent the data and other corresponding attributes, for example tandem mass spectra and corresponding attributes comprising DATA_FILE, API_EXPERIMENT_NO, LCMS_SCAN_NO, LCMS_LCTIME, OBSERVED_MZ, OBSERVED_Z, TANDEM_LCMS_MAX_ABUNDANCE, TANDEM_LCMS_PRECURSOR_ABUNDANCE, TANDEM_LCMS_SNR, LCMS_SCAN_MGF_NO, or additional key-value pairs that represent data for analyte identification or analysis.

Third, the file contents of the corresponding third-party data file, such as a *.dat file, can be read for each key-value(s) pair. The third-party data file can contain data obtained by an instrument analysis workstation, for example a *.dat file contains a list of value pairs (mz, abundance) observed as a centroid tandem mass spectrum.

Fourth, a flat file can then be written out in the desired universal file format, such as an *mgf file format. An example of an *.MGF file segment corresponding to a tandem spectrum is as follows.

BEGIN IONS
PEPMASS=OBSERVED_MZ
CHARGE=OBSERVED_Z
TITLE=file: DATA_FILE scan: LCMS_SCAN_NO lctime:
LCMS_LCTIME max_int:
TANDEM_LCMS_MAX_ABUNDANCE
MZ ABNDANCE
MZ ABNDANCE
MZ ABNDANCE
. . .
END IONS
. . .

Fifth, using a database project file, such as a MySQL Object, the array of key-values pair attributes can be inserted into the corresponding database, such as Pipeline MySQL Database given API_EXPERIMENT_NO as the primary_key.

Consistent with the specification, alternative input file types containing other data types will produce output files consisting of different attributes.

Some embodiments comprise methods of automated mass spectrometry analysis and computer systems configured to determine a correction to mass spectrometry values, such as for tandem mass Spectra MS1 values. Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

One or more methods described herein comprise determining a correction to a mass spectrometry value. A mass spectrometry value correction process can comprise receiving a data file comprising changing one or more data values in the file, and saving the changes. For example, the process may comprise computing a correction to tandem mass Spectra MS1 value. Data values often are tandem mass spectra precursor ion assigned MZ and CHARGE_STATE. For example, the data values can be assigned by another process, such as a precursor ion assignment generated by one or more peak detection processes described herein (e.g., peak picker).

A mass spectrometry value correction process can comprise receiving an input file, generating an output file containing the corrected data. The input file may be a *.mgf file, or any other file containing data to be corrected. The output file may comprise a corrected file, such as a corrected *.mgf file. The corrected *.mgf file may be renamed as the original *.mgf file.

A mass spectrometry value correction process can use one or more constants. In one aspect of the method, a constant MZ_TOLERANCE_PPM is used. MZ_TOLERANCE_PPM is often 15. In some cases, MZ_TOLERANCE_PPM can be another value, such as a value no more than 1, 2, 5, 10, 15, 20, 25, 30, 50, or no more than 100. In some cases, MZ_TOLERANCE_PPM is at least 1, 2, 5, 10, 20, 25, 30, 50, or more than 50.

An example of a workflow for a mass spectrometry value correction process is provided as follows. For example, an input file can be provided, such as into a memory. For example, the input file can be a *.mgf file. Second, file contents from memory can be parsed into an array of key-value(s) pairs that represent tandem mass spectra and corresponding attributes; for example DATA_FILE, API_EXPERIMENT_NO, LCMS_SCAN_NO, LCMS_LCTIME, AGILENT_OBSERVED_MZ, AGILENT_OBSERVED_Z, LCMS_SCAN_MGF_NO. Third, using a database object, such as MySQL Object the corresponding *.mgf file PeakPicker precursor ion attributes LCMS_LCT1ME, API_OBSERVED_MZ, API_OBSERVED_Z, LCMS_SCAN_MGF_NO can be retrieved.

Fourth, for each tandem mass spectra represented in the *.mgf file, the OBSERVED_MZ(s) can be compared. If the absolute value of ((API_OBSERVED_MZ AGILENT_OBSERVED_MZ)/AGILENT_OBSERVED_MZ*1e6) is greater than MZ_TOLERANCE_PPM, then AGILENT_OBSERVED_MZ can be replaced with API_OBSERVED_MZ.

Fifth, for each tandem mass spectra represented in the *.mgf file OBSERVED_Z(s) can be compared; if API_OBSERVED_MZ does not equal AGILENT_OBSERVED_Z, then AGILENT_OBSERVED_Z can be replaced with API_OBSERVED_Z.

Sixth, the data can then be outputted to a flat file format, such as an *mgf file format. An example of a Z and MZ corrected *.MGF file segment corresponding to a tandem spectrum is:

BEGIN IONS
PEPMASS=API_OBSERVED_MZ
CHARGE=API_BSERVED_Z
TITLE=file:DATA_FILE scan:LCMS_SCAN_NO
lctime:LCMS_LCTIME
max_int:TANDEM_LCMS_MAX_ABUNDANCE corr:mz&z
MZ ABNDANCE
MZ ABNDANCE
MZ ABNDANCE
. . .
END IONS
. . .

Seventh, using a database object such as MySQL Object, array of corrected key-values pair attributes can be updated to the corresponding database such as Pipeline MySQL Database given API_EXPERIMENT_NO as the primary_key.

Additional processes with different variable for calculating corrections to data, such as tandem mass data, can also be consistent with the specification.

Some embodiments comprise methods of automated mass spectrometry analysis and computer systems configured to determine the proteomic false discovery rate for assigned peptides, such by using search engine expectation values. Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

One or more methods described herein comprise determining a rate of false peptide assignments. A process for determining a rate of false peptide assignments can be performed for a given population of mass spectrometry values, such as a population of tandem mass spectral search engine's score and/or expectation values.

A process for determining a rate of false peptide assignments can comprise receiving an input comprising an ordered list of search engine scores or expectation values in descending order from both a TRUE_POPULATION and NULL_POPULATION. The TRUE_POPULATION can comprise peptide matches and corresponding expectation values calculated from a protein sequence database with amino acids listed from N-terminal end to the C-terminal end. The NULL_POPULATION can comprise peptide matches and corresponding expectation values calculated from a protein sequence database with amino acids listed in reverse or from the C-terminal end to the N-terminal end. The process can comprise providing an output comprising one or more expectation values associates with a False Discovery Rate (FDR) p-value. The p-value can be between 0 and 1. In some cases, the p-value is at most 0.1, 0.2, 0.5, 0.7, or at most 1.0.

A process for determining a rate of false peptide assignments can use one or more constants. The process can use a first constant RETURNED_FDR_VALUES.

RETURNED_FDR_VALUES often is 0.1, 0.15, 0.2, 0.25, 0.3. In some cases, RETURNED_FDR_VALUES can comprise a different value, including an alternative list of one or more p-values. In some embodiments, RETURNED_FDR_VALUES comprises one or more FDR p-values at least 0 and no more than 1.

An example of a workflow for a process for determining a rate of false peptide assignments is provided as follows. The process comprises one or more steps to output a file comprising one or more expectation values for a given measurement of the false discovery rate, such as FDR. First, the file contents of a search engine results file can be read into a memory as an object representing a true population. For example, the file contents of Proteomic Search Engine results *.fasta.csv file can be read into memory as Object TRUE_POPULATION. Second, the file contents of a search engine results file can be read into the memory as an object representing a null population. For example, the file contents of Proteomic Search Engine results *.rev.fasta.csv file can be read into the memory as Object NULL_POPULATION.

Third, an expectation value for a given False Discovery Rate using a method such as the Benjamini-Hochberg-Yekutieli method can be computed. Fourth, the calculated expectation value for each RETURNED_FDR_VALUES can be looked up, and the calculated value can be placed in an array of key-value pairs. Fifth, using a database object such as a MySQL Object, an array of key-values pair attributes can be inserted into a corresponding database, such as Pipeline MySQL Database given API_EXPERIMENT_NO as the primary_key.

Other false discovery methods consistent with the disclosure can also be used.

Some embodiments comprise methods of automated mass spectrometry analysis and computer systems configured to improve protein identification, such as comprising execution of a target decoy approach to protein identification. Practice of the methods herein and implementation of the computer systems herein enable or facilitate automated mass spectrometric analysis, such that in some cases human interaction or oversight of the methods is optional or not required. Often, practice of the methods herein and implementation of the computer systems herein facilitate data analysis in no more than 8 hours, 4 hours, 2 hours, 1 hour, 30 minutes, 20 minutes, 15 minutes, 10 minutes 5 minutes 1 minute or 30 seconds. In some cases data analysis occurs in no more than 1 minute.

One or more methods described herein comprise a process for improving identification of proteins, such as increasing a number of proteins identified in a sample. The methods can be performed to increase the number of analytes identified from the data acquired on an analytical instrument platform, such as LCMS, MALDI-TOF or any other instrument that can be used to identify analytes. A process for increasing a number of identified proteins can prioritize specific elements of data for analysis, to facilitate identification of an increased number of proteins in a sample while maintaining desired overall analysis time. Existing analytical instruments can tend to target the same features across multiple runs of the same sample, thereby reaching a plateau on the number of proteins identified in that sample (e.g., auto-MS/MS feature on some analytical instruments). A process for increasing a number of identified proteins as described herein can comprise selection of particular target features, so as to facilitate improved protein identification (e.g., for MS2 spectrometry). For example, the process may comprise requesting the instrument to perform MS2's on specific features not previously targeted, such that significantly more proteins can be identified. The process can comprise prioritizing MS1 features for targeting to achieve increased protein identification from proteomic samples.

A process for increasing a number of identified proteins can comprise a series of steps to generate a prioritized target list. First, features with poor MS2 performance can be excluded, such as those with undesirable Z. Features with poor MS2 performance often are features with Z=1, or Z>5. In some embodiments, a feature can be excluded if a Z score is no more than 1, or at least 1, 2, 3, 4, 5, 10, 20, 50, or more than 50. Second, features with m/z values which may not return good scores can be excluded. For example, features with m/z<350 can be excluded. In some embodiments, a features can be excluded if an m/z is no more than 50, 75, 100, 200, 300, 400, 500, 750, 1,000, 2,000, 5,000, 10,000, or no more than 100,000.

Third, features can be clustered by neutral mass to form Neutral Mass Clusters (NMC) at a given retention time. An NMC can correspond to a single peptide. Fourth, NMC's can be prioritized based on a set of factors intrinsic to the cluster, which can include any previous MS2-based identifications (e.g., as outlined below). Fifth, a single target for each NMC can be generated, which specifies a target charge state, elution time, collision energy, and acquisition time. Sixth, NMC's which have been targeted twice will or achieved a high-confidence identification (e.g., score greater than 20) can be assigned the lowest priority. In some embodiments a high-confidence score is at least 5, 10, 20, 50, 75, 90, or more than 90. Seventh, the final target list can be generated in a manner which both achieves high-priority targeting and limits the number of targets to match the instrument's maximum target acquisition rate. Features can be targeted to within a time period, for example, 6 seconds of their LCMS peak, to facilitate high abundance.

MS1 features can be grouped together based on neutral mass within a small retention time window to form NMC's. These NMC's can be prioritized to create a target list, with a single of the NMC's charge states selected for targeting in a given injection. The NMC priority can be determined by one or more factors such as abundances of its charge-state features, the amount of information already determined about the NMC's identity, or other factors consistent with the specification. For example, NMC priority can be determined by OMSSA scores and feature abundance. First, OMSSA scores of any previously performed MS2's on features within the NMC can be considered. The higher the previously found scores can be indicative of the more information already acquired, which can lower its priority. Second, feature abundances can comprise its charge-state features, for example, since low-abundance features tend not to good MS2 spectra.

NMC's can be prioritized based on the amount of information already determined about the NMC's identity. If an NMC has less information available, its priority can be higher. The information available for an NMC can be determined for each assigned molecular feature as follows: mfeScore=0 for molecular features not previously attempted, mfeScore=1 for molecular features previously attempted and not scored, mfeScore=highest score to date for features previously attempted and scored, mfeAbundance=average across all injections of the feature's MS1 peak_height, low_mass_contamination=ratio of highest MS1 value between −2.00 and −0.25 AMU offset from the target's mz, divided by the MS1 value at the target mz (this quantity can reflect the amount of contaminating analytes expected to be passed into the collision cell from m/z's lower than the target's), well_ratio=ratio of the MS1 value at (mz)+1/(2z) to the MS1 value at mz, where z is the charge of the analyte (this quantity can reflect the amount of contaminating analytes present in the collision cell), or any other acquired features consistent with the specification.

Molecular features not previously targeted and with no existing peptide matches can be given zero mfeScore and therefore the highest priority. Features which have been targeted previously but not scored can be next highest in priority, followed by features which have been scored. Features which have been scored can receive lower priority, since there may be less information to be gained by targeting them. NMC's can be assigned abundances given by the average abundance of their highest-abundance charge state feature.

After molecular features have been prioritized as described herein, predetermined criteria can be used to generate an NMC list in a series of four tiers according to the following criteria and ranked. A first of the criteria can comprise values of ms1p. For example, the first of the criteria can comprise ms1p<0.33. In some cases, other values can be used, such as at least 0.05, 0.1, 0.15, 0.2, 0.3, 0.4, 0.5, 0.75, or more than 0.75. A second of the criteria can comprise max(mfeAbundance). For example, the second of the criteria can comprise max(mfeAbundance)≥2000. In some cases, other values can be used, such as at least 100, 200, 500, 1000, 2000, 5000, 10,000, or more than 10,000. A third of the criteria can comprise max(low_mass_contamination) and max(well_ratio), including max(low_mass_contamination)<1, and max(well_ratio)<0.1. In some cases, max(low_mass_contamination) can be no more than 0.1, 0.2, 0.5, 0.9, or no more than 1. In some cases, max(well_ratio) can be no more than 0.05, 0.1, 0.2, 0.5, 0.7, or no more than 1.

NMC's can be sorted into the four tiers according to how many of the first, second and third criteria are met, for example, tier 1 can be populated by NMC's passing all three criteria, tier 2 can be populated by NMC's passing two of the three criteria, tier 3 can be populated by NMC's passing one of the three criteria, and tier 4 can be populated by NMC's passing none of the criteria. In some cases, NMC's may be sorted into tier 4 for if one or more of the following is satisfied: the NMC has passed none of the criteria, has max(mfeScore)≥20, has been targeted in two or more LCMS experiments. In some cases, other max(mfeScore) can be used, such as at least 1, 5, 10, 20, 50, 100, or more than 100. Additional tiers can used in examples that involve more than three criteria, consistent with the specification.

Within each tier, NMC's can be prioritized (1) by score (e.g., lowest scores receiving highest priority), and then (2), within each fixed score ranking, by NMC abundance (e.g., with higher abundance NMC's receiving highest priority). This prioritization method can facilitate labeling those NMC's which have not been previously targeted with highest priority, and those with existing identifications with lower priorities the higher confidence in the identification (e.g., the higher the score). Other criteria and variables can be used to prioritize NMCs, consistent with the specification.

For each NMC in the resulting targeting list, a target methodology can be assigned. The methodology comprises one or more decisions and variables. The target methodology can determine how the target is acquired. The methodology can comprise one or more of (1) LC time of the target (such as within 6 seconds of the LCMS peak retention time), (2) which charge state to pursue, (3) the collision energy to apply, and (4) the acquisition time, or additional elements used to assign a target methodology. For which charge state to pursue, if a z=2 feature is present then it can be chosen unless another feature has more than twice the abundance of that feature, in which case the highest abundance feature can be chosen. Alternatively if no z=2 feature is present, then the highest abundance feature can be chosen. For the collision energy to apply, the collision energies can be chosen based on one or more formulas obtained through testing, comprising: (1) (Z<=2) CE=−9.77+0.045*mz, (2) (Z=3) CE=−8.88+0.0388*mz, (3) (Z>=4) CE=−9.58+0.041*mz, or other formulas consistent with the specification for calculating collision energies. For the acquisition time, the MS2 acquisition time can be set to Min(1500, Max(125, 3E6/abundance)), for example in milliseconds. The result can be a single target specified per NMC.

For generating a target list, due to the finite time over which the MS instrument can perform MS2's, the list of targets produced may not be compatible with a single injection. Sub-selection of targets can in some embodiments be required. One or more processes used for sub-selection of targets can comprise application of one or more facts. A first fact can comprise the fact that the instrument can attempt to perform a single 250 ms MS1 scan per second, as specified in the acquisition method. In some aspects, the MS1 scan time is no more than 1, 2, 5, 10, 20, 50, 100, 200, 500, 1,000, 2,000, 5,000, or no more than 10,000 ms. In some embodiments the MS1 scan time is at least 1, 2, 5, 10, 20, 50, 100, 200, 500, 1,000, 2,000, 5,000, or more than 5,000 ms. If an MS2 scan is longer than 750 ms, then this rate of MS1's (such as 250 ms) may not be achieved. However, approximately 25% of the instrument's time for MS1's can be budgeted given this specification for MS1 acquisition rates. A second fact can comprise the fact that MS2 acquisition time can be adjusted to a range, such as between 125 ms and 1500 ms, based on feature abundance. In some cases this range can be defined as having an upper limit of at least 1, 5, 10, 20, 100, 200, 500, 1,000, 2,000, 5,000, or more than 5,000 ms, and a lower limit of no more than 1, 5, 10, 20, 100, 200, 500, 1,000, 2,000, or no more than 5,000 ms. A third fact can comprise the fact that each target can have an associated range of retention times for targeting, such as within 6 seconds, or within 1, 2, 3, 4, 5, 6, 7, 8, 9, 10, or 20 of the feature's average LCMS peak retention time. A fourth fact can comprise the fact that each MS2 target can be specified within the list along with an associated range of target retention time. The instrument control software can control if, when, or how often within the specified interval the target will actually be acquired. This control may be performed on the fly. The MS2 prioritization process can be flexible, for example being capable of handling missed opportunities in which a target was not acquired by re-targeting them on a later injection.

One or more processes can be performed to slot, in priority order, the desired targets into the target list while remaining within the instrument's MS2 budget. An example of such a process can comprise first, creating an array of floating point values of length equal to the number of seconds within the injection divided by a constant, such as 1.75 seconds. Each of these values can be set to a time budget, such as 1500 for MS2's within each allocation slot. Each of these 1.75 second bins can be meant to account for the time of one MS1 scan (for example, 250 ms) together with a time allotment for MS2 scans, such as 1500 ms, for example allowing the process to budget for the potentiality of 1500 ms MS2 scans while often more than this ratio of time are used for MS1's.

Second, the tiered NMC list can be iteratively processed starting with Tier 1. The substituent tier often can be exhausted before proceeding to the next tier. Within each tier, molecular features can be iteratively budgeted in order from highest to lowest priority within the tier using one or more steps. In one example, budgeting can comprise 1) for a given target, the array element closest to the center of the target's temporal acquisition interval which has remaining MS2 time can be found and budget at least as large as the acquisition time of the target, 2) if no such array element is available, then the target can be not added to the final target list (e.g., it is outside of the available time budget), and 3) if an array element is found, then the element's value can be reduced by the acquisition time of the target. This target can be added to the final target list.

Time budgeting in some embodiments may comprise different steps and time ranges, consistent with the specification.

Consistent with the specification, alternative inputs, outputs, constants, processes, or other components may be used to align analyte feature data across samples.

Some embodiments of the workflow disclosed herein comprise incremental clustering of mass spectrometry data into previously or concurrently developed datasets. Accurate, automated, fast mass spectrometry data analysis as disclosed herein comprises the analysis of mass spectrometry data so as to generate processed study data, such as data for which mass signals have been clustered across time of flight runs and across various predicted peptide fragments of a given protein so as to generate protein abundance measurements, for which fill-in analysis has been performed to smooth data in light of potential miscalling errors, particularly errors that arise in regions of a mass spectrometry output where mass signals are particularly dense, and in some cases for which data has been normalized across individual mass spectrometric outputs. Even in automated data analysis workflows, this process is computationally intensive and often slow.

To facilitate this analysis, some approaches involve batch analysis, whereby multiple datasets are aggregated, subjected to at least some of the analyses mentioned above or elsewhere disclosed herein. Batch analyses concentrate the computationally intensive steps of the data analysis workflow into discrete segments of the workflow.

A drawback of such an approach is that new data is not easily incorporated into processed datasets as it is generated. Rather, data must be amassed into batches, and then subjected to de novo analysis of the new batches and the prior datasets to produce an integrated, updated processed dataset. Although batch analysis concentrates computationally intensive steps of the data analysis workflow into discrete segments of the workflow, the computational burden of introducing a new batch remains substantial, as previous datasets and batched new datasets must be reanalyzed concurrently.

In addition, as datasets are analyzed in batches, processed datasets are not generated until the end of data entry. Accordingly, the impact of a particular mass spectrometric run on the processed dataset is not easily evaluated in isolation.

Disclosed herein is an alternative to batch analysis to generate processed datasets. Through the disclosure herein, processed datasets are continually or iteratively updated as new data is added, rather than processing batches at the end of data input. That is, as part of data input, a dataset or datasets are subjected to, for example clustering blank-filing and normalization, and are incorporated into the processed dataset ‘master map’ comprising survey-wide assessments of all data entered. Rather than waiting for batch aggregation, individual or smaller sets of data are added iteratively, in an ongoing manner as data is entered.

As a consequence of this approach, the impact of a single dataset addition is easily assessed as it is entered, rather than having the set bulked and added only with the other datasets being processed. Consequently, modifications to a data input protocol, to sample collection or to sample processing may be made in light of data processing outcomes, as the data input, sample collection or sample processing is occurring, in some cases in real time. Such iterative assessments facilitate refinement of an ongoing study, where batch analysis precludes conclusions as to input data until the input and individual data entry and processing steps are complete.

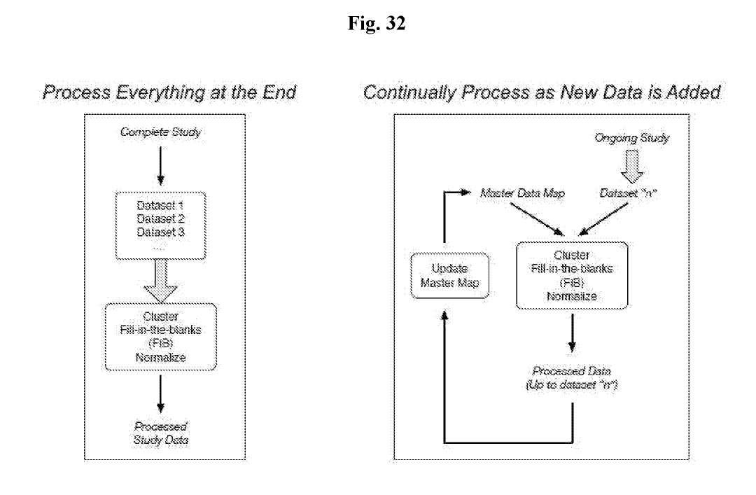

At FIG. 32, one sees a side-by-side comparison of workflows for batch analysis (left) and concurrent analysis (right). Under a batch analysis regimen (left), a study is completed, datasets are entered completely and processed by, for example, subjecting the datasets to clustering, blank-filling and normalization, and only then is the batch integrated with previous master map data to form a new master map dataset. New data is not easily incorporated without reassessment of previously analyzed data, and processing does not occur until a study is completed.

Under a concurrent analysis data is continually processed as new data is added. A particular dataset, ‘n,’ is collected as part of an ongoing study and entered for analysis. The dataset n is processed by, for example, subjecting the datasets to clustering, blank-filling and normalization, independent of whether subsequent datasets are being entered.

Dataset n is then entered into a master map of entered datasets, previously including datasets 1 to dataset ‘n−1’. The dataset is incorporated into the master set, and the master set is configured for addition of subsequent datasets, such as ‘n+1,’ as they are generated. Dataset assessment and integration into a master set is concurrent with data generation rather than being delayed unlit the formation of a sufficiently large batch for group processing.

### Biomarker Database Development, Biomarker Sources, and Characteristics

Some methods, databases and panels relate to health assessment, heath categorization or health status assessment relying upon marker database development.

Marker data are obtained from at least one source as disclosed herein. A focus of the disclosure herein is biomarkers obtained from fluids, such as blood, plasma, saliva, sweat, tears and urine. Particular attention is paid to blood, and to plasma extracted from a blood sample, such as prior to drying the blood sample. However, alternative biomarker sources are contemplated and are consistent with the disclosure herein.

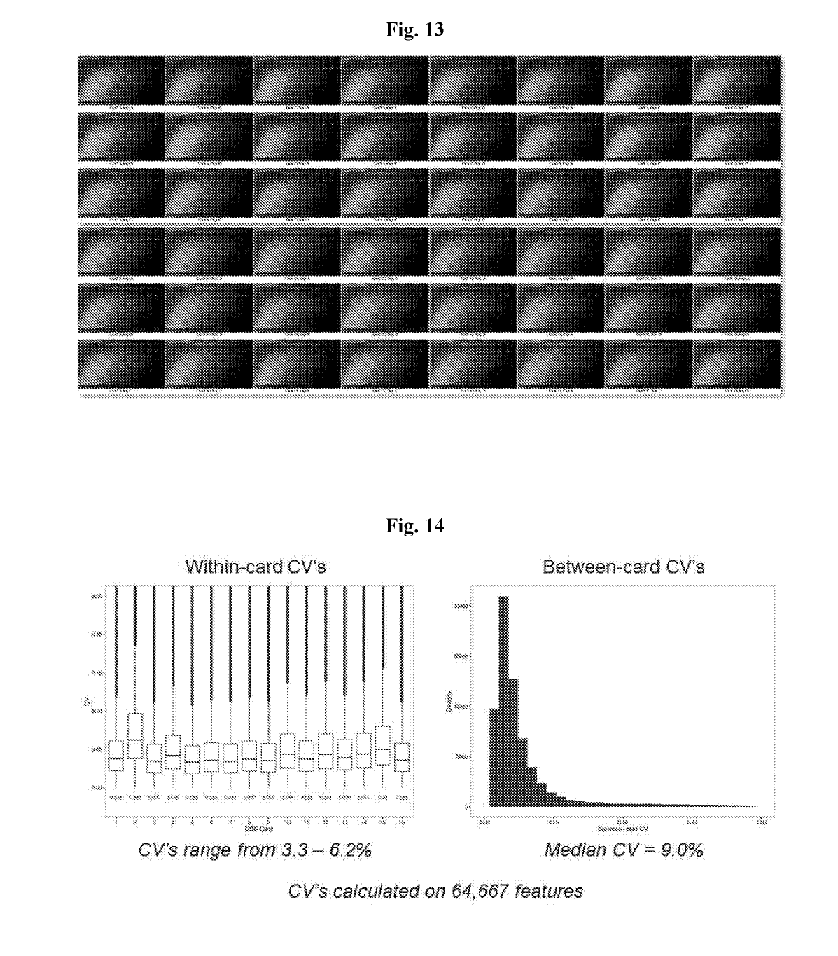

Marker sources include but in some cases are not limited to proteomic and non-proteomic sources. Examples of sources of markers include age, mental alertness, sleep patterns, measurement of exercise or activity, or biomarkers that are readily measured at the point of collection, such as glucose levels, blood pressure measurements, heart rate, cognitive well-being, alertness, weight, are collected using any number of methods known in the art. Some marker sources are indicated in, for example, FIG. 27. Exemplary biomarker sources include circulating biomarkers in a blood or plasma sample or biomarkers obtained from breath aspirate that are quantified, either relatively or absolutely, through mass spectrometric approaches or using antibodies, or other immunological or non-immunological approaches. Examples of raw data obtained from such sources is given in FIGS. 13, 26 and 28.

In some examples, biomarker data sources include physical data, personal data and molecular data. In some examples, physical data sources include but are not limited to blood pressure, weight, heart rate, and/or glucose levels. In some examples, personal data sources include cognitive well-being. In some examples, molecular data sources include but are not limited to specific protein markers. In some examples, molecular data includes mass spectrometric data obtained from plasma samples obtained as dried blood spots and/or obtained from captured exudates in breath samples. One example of raw mass spectrometric data generated from captured exudates in breath is given in FIG. 27. In some examples, biomarker and other marker data from multiple sources are integrated as part of a multi-source marker regimen, and depicted in FIG. 29.

Additionally, some biomarkers are informative of the environment from which a sample is taken, such biomarkers include, weather, time of day, time of year, season, temperature, pollen count or other measurement of allergen load, influenza or other communicable disease outbreak status.

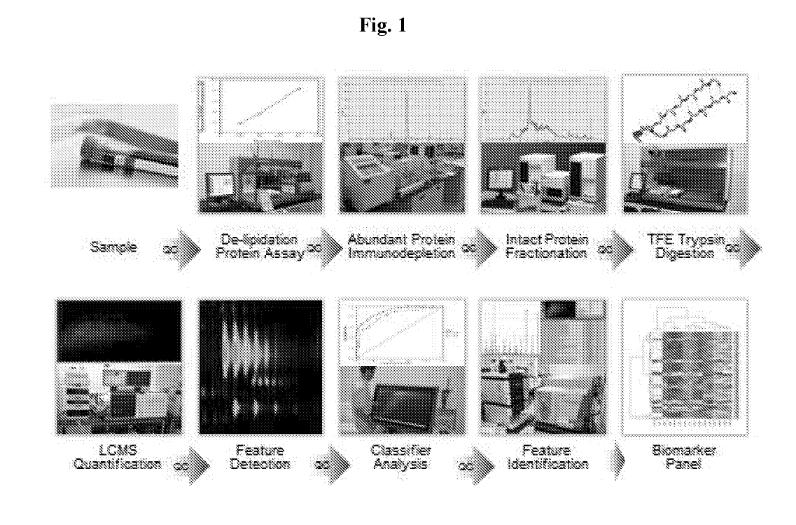

Biomarker-based data in some cases comprises large amounts of potentially relevant biomarkers. In particular, databases disclosed herein comprise in some cases at least 10, at least 50, at least 100, at least 1,000, at least 5,000, at least 10,000, at least 20,000 or more obtained from a single sample, such as a readily obtained sample deposited as a blood spot on a solid surface, such as seen in FIG. 1. Collecting biomarker data from blood spots, alone or in combination with other readily available sources of biomarker or other marker data, dramatically facilitates database generation. Samples are collected in some cases remote from a health facility or laboratory, and are stored and transmitted without costly refrigeration. Nonetheless, as indicated in the description including the figures and examples herein, large quantities of biomarker data are obtained, facilitating database generation.

Databases are variously developed from a plurality of individuals or sample sources collected at a single time point or a plurality of time points, at one sample per individual or multiple samples per individual, collected at one or at multiple time points from one or multiple individuals. In some cases databases are developed from a single individual or other single sample source through repeated sampling and biomarker processing over time, so as to produce a ‘longitudinal’ or temporally progressing database. Some databases comprise both a plurality of individuals and a plurality of collection time points.

In some cases, an individual or a sample taken from an individual at a particular time is associated with a health condition or health status for that individual at that time. Thus, biomarkers or other markers obtained from a sample are associated with a health condition or health status, such as presence, absence, or a relative level of severity of a disorder.

Data is often collected and analyzed over time. Groups of markers that change over time and are linked may be monitored together, for example, markers implicated in glucose regulation such as glucose levels, mental acuity, and patient weight. In some examples, differences in these markers may be indicative of disease states or disease progression. Similarly, in some cases data is collected in combination with administration of a treatment regimen or intervention, such that data is collected both before and after a treatment such as a pharmaceutical treatment, chemotherapy, radiotherapy, antibody treatment, surgical intervention, a behavioral change, an exercise regimen, a diet change, or other health intervention. Data analysis can indicate whether a treatment regimen was successful, is impacting a biomarker profile such as reducing marker levels or slowing to health decline-related change in biomarker levels, or otherwise continues to be relevant to a patient. In some examples, a report detailing the patient's markers can inform a medical professional.

Biomarker levels that vary in concert with differences in health condition or health status are in some cases selected for validation as individual indicators or as members of panels indicative of health condition or health status. Often, individual markers are identified that correlate with health condition or status, but overall predictive value is improved when multiple markers, particularly markers that do not strictly co-vary, nonetheless are independently predictive of health status.

In some cases the biomarkers are further identified as to protein source, such that protein specific analysis is performed. The protein identifies are analyzed, for example so as to shed light on a biological mechanism underlying a correlation between a biomarker level and a health condition or status.

When the protein or other biomarkers are known, their detection in a mass spectrometry analyzed dataset is facilitated in some cases by the introduction of labeled biomarkers into a sample prior to mass spectrometric analysis. Labeled markers are markers such as heavy isotope labeled biomarkers that are detectable independent of the biomarker mass spectrometry labeling approach, and that migrate in mass spectrometry analyses at a repeatable, predictable offset from a native or naturally occurring biomarker in the sample. By identifying the labeled markers in a mass spectrometric output, and in light of the known offset of the native biomarker relative to its labeled counterpart, one can readily identify the expected position and size of a biomarker spot on a mass spectrometric output. Such labeling facilitates accurate, automated calling of large numbers of biomarkers in a mass spectrometric sample, such as 100, 200, 300, 400, 500, 600, 700, 800, 900, 1,000, or more than 1,000 biomarkers in a sample.

Biomarkers that map to known proteins are often examined as to whether their measurement using immunology-based methods yields results that are similarly informative as compared to mass spec data. In such cases, the biomarkers are in some cases developed as constituents of stand-alone panels for the detection or assessment of a specific health condition or health status, such as a cancer heath status (e.g., colorectal cancer health status), coronary artery health status, Alzheimer's or other health condition. Such stand-alone panels are in some cases implemented as kits to be used in a medical or laboratory facility, or to be implemented by providing samples for analysis at a centralized facility.

In some cases, however, biomarkers retain predictive utility independent of any information regarding a protein from which they are derived. That is, biomarkers identified as mass spectrometric signals having levels that vary in correlation with the presence or severity of a health condition or health status may in some cases retain a utility as markers on their own. Even without information regarding a biological mechanism underlying the correlation (as may be obtained by identifying a protein correlating to the marker and by examining the biological function of the protein) the biomarker in itself, as it appears on the mass spectrometric result, possess utility as a biomarker alone or in combination as indicative of a health status or condition or level of severity. Such biomarkers often rely upon mass spectrometric detection and may not in all cases be conducive to development as immunologically based stand-alone assays. However, they remain useful as stand-alone markers or as constituents of detection approaches comprising mass spectrometry-based detection at least some biomarkers in a panel.

In some cases, even when a biomarker identity is not known, one can generate a labeled biomarker that migrates at a predicted offset relative to the unidentified relevant biomarker. Thus, even in the absence of the biomarker's identity, labeled offset biomarker approaches can be used to facilitate high-throughput collection of this type of marker.

Biomarker databases developed hereby often possess a number of interrelated characteristics. Firstly, the databases can harbor from less than 20 to 1,000s or 10,000s of biomarkers per sample, and often further comprise non-biomarker data such as glucose levels, age, caloric intake, sleep patterns, blood pressure measurements, mental acuity tests or other non-sample marker data as disclosed herein.

Accordingly, signals can be derived from these biomarker datasets by assembling individual biomarker and other markers into panel that provide statistical signals that are strong enough for medical relevance even when the individual markers do not on their own generate statistically relevant or medically reliable signals.

Secondly, biomarker databases developed herein are readily generated from easily obtained starting material. Samples yielding at least 10, at least 50, at least 100, at least 1,000, at least 5,000, at least 10,000, at least 20,000, at least 30,000 or more markers are obtained from dried blood spots or other blood stabilization approach such as sponge collection, and collected often remote from a medical or laboratory facility. Biomarkers are also readily obtained in substantial numbers from collected breath aspirate or from other fluid or tissue sample.

The adoption of such easily obtained starting material facilitates both generation of large numbers of biomarkers from a single sample, but also facilitates processing of multiple samples, either from multiple individuals in at least one cohort, or from a single individual over multiple points in a time course, or from multiple individuals at multiple time points. The ease with which samples are collected and processed exponentially increases dataset size.

Thirdly, because biomarker databases are generated readily from easily obtained and stored samples, because such large numbers of biomarkers are assayed from a single sample, and because samples are readily obtained from single individuals over multiple times of a time course, one is able to investigate changes over time in an individual's biomarker profile on a scale comparable to that of one's genomic or exomic nucleic acid sequence information, and at the same time to detect changes in this dataset indicative of changes in health status. Nucleic acid databases are valuable sources of personalized medical information, but are ill-suited to detect changes that occur over time, such as changes leading to mutations in genes implicated with changes in health status or health category. Cancer mutations, for example, often occur only in a tiny subset of cells in an individual. Untargeted genomic sequencing efforts do not detect these mutations at any reliable frequency. Thus, inherited oncogenes are readily detected, but changes that may impact health status are unlikely to be detected in a general genomic sequencing effort.

Using databases generated as disclosed herein, biomarkers are obtained having a level of information comparable to the relevant information in a genomic sequence (that is, comparable to the subset of genomic information that varies among individuals and is relevant to health status or health classification). However, additionally, as a genomic or other change occurs in an individual that may impact health status or health classification, these changes are readily detected in real time in the generation of ‘longitudinal’ or temporally iteratively sampled databases as disclosed herein. Thus, unlike comparable genomic databases, the biomarker databases as disclosed herein capture signals that are reflected in differential levels of protein or other biomarkers as these changes occur. Databases as disclosed herein are consistent with and compatible with genomic information, and genomic information can be included as marker information for databases as disclosed herein to be considered in making health status or health categorization determinations, but unlike genomic data in isolation, biomarker databases as disclosed herein incorporate temporal information relating to health status or health condition progressions over time, such that one can identify not only a risk of developing a health condition, but to identify the condition in its early stages of development, thereby facilitating early treatment precisely when it is suitable for a given condition.

### Biomarker Database Uses

Biomarker databases as disclosed herein have at least two related uses in health assessment. Firstly, databases are used to identify markers that correlate with health status in among two cohorts that vary in that health status. Cohorts can comprise single sample marker information, or more often, marker data including biomarker data obtained from multiple members of each of at least two cohorts, sharing at least one common health status within each cohort. Biomarker or other markers that correlate with health status or health categorization, either alone or in combination, are identified from among the at least 10, at least 50, at least 100, at least 1,000, at least 5,000, at least 10,000, at least 20,000, at least 30,000 or greater than 30,000 biomarkers in the database. Biomarkers or other markers may be effective distinguishers of the cohorts alone, or more often in combination with other biomarkers or other markers to form panels that generate signals of stronger statistical relevance or predictive AUC values.

Biomarkers may correlate to or map to proteins of known function in a health status or health condition, or may correlate or map to proteins of unknown function. Alternately, biomarkers in some cases are not mapped to a known protein but are nonetheless useful as mass spectroscopy-based markers or distinguishers of health status or health category. Biomarkers of particular interest may subsequently be mapped to a protein, without impacting the biomarker's use in mass spectrometric analysis.

Biomarkers that are mapped to a particular protein are in some cases developed as health status or condition specific panels. These panels are consistent with mass spectrometric information, but in some cases are developed for independent, targeted use, for example in immunological assays. These assays are implemented through the use of stand-alone kits comprising immunological reagents for the detection of biomarker proteins, or through the delivery of samples to a facility for sample analysis.

Secondly, databases as disclosed herein are used for the ongoing temporal monitoring of at least one individual from whom database samples are obtained. In this use, an individual or individuals (such as an individual or a cohort of individuals subjected to a common treatment regimen, or a single individual or cohort of individuals for which no health status hypothesis is initially present) are subjected to ongoing sampling, and databases are developed ‘longitudinally’ or over time. Changes in biomarker levels are observed over time, and when the biomarkers are mapped to proteins implicated in at least one particular health condition or health status, the health status or health condition is identified as potentially being in flux in the individual or population. These uses are not mutually exclusive. Some databases are readily used for both aims. A significant change between measurements may comprise a change of at least 10%, or at least 1%, 2%, 5%, 10%, 20%, or at least 50% of a marker implicated in a disorder. A significant change between measurements may comprise a change of at least 10%, or at least 1%, 2%, 5%, 10%, 20%, or at least 50% of a plurality of markers implicated in a common disorder.

Additionally, databases are in some cases used to cluster patients into groupings independent of any current condition or category. Patients are grouped largely or solely based upon biomarker profiles, and are then observed for commonalities both at the time of sample collection, and retrospectively over time. As a health condition changes in a member of a given grouping, the remaining members of the grouping may be cautioned to assay for the health condition. Alternately, the member may have his or her biomarker profile reassessed, so as to determine whether the individual remains in said grouping.

Ongoing monitoring using the disclosure herein is implemented through a number of approaches, such as the following. An ongoing health monitoring protocol is implemented for an individual by measuring biomarkers from a wide diversity of potential sources, as indicated in FIG. 27. In some examples, biomarker data sources include physical data, personal data and molecular data. In some examples, physical data sources include but are not limited to blood pressure, weight, heart rate, and/or glucose levels. In some examples, personal data sources include cognitive well-being. In some examples, molecular data sources include but are not limited to specific protein markers. In some examples, molecular data includes mass spectrometric data obtained from plasma samples obtained as dried blood spots and/or obtained from captured exudates in breath samples. One example of raw mass spectrometric data generated from captured exudates in breath is given in FIG. 27. In some examples, biomarker and other marker data from multiple sources are integrated as part of a multi-source marker regimen, and depicted in FIG. 29.

Data is collected and analyzed over time. Groups of markers that change over time and are linked may be monitored together, for example, markers implicated in glucose regulation such as glucose levels, mental acuity, and patient weight. In some examples, differences in these markers may be indicative of disease states or disease progression. For example, glucose levels are found to vary over the course of the protocol. Glucose levels are observed to be successively less regulated, but not at levels that would on their own indicate diabetes. Biomarkers correlating to glucose regulation, and implicated in diabetes, are found to change in levels monitored through the course of the monitoring. It is observed that mental acuity is affected in a manner that correlates with blood glucose levels. It is also observed that the magnitude of these changes scales roughly with an increase in patient weight. In this example, each of these markers shows some change, but none of these markers individually generates a signal strong enough to lead to a statistically significant signal indicative of progression toward diabetes. Nonetheless, the aggregate signal generated by a multifaceted analysis involving markers from a diversity of sources, including biomarkers from patient dried blood samples, strongly indicates a pattern trending toward the onset of diabetes.

### Labeled Biomarker Reference Molecules

Some mass spectrometric or other approaches herein involve labeled biomarker reference molecules or standards, variously referred to as mass markers, reference markers, labeled biomarkers, or otherwise referred to herein. Such standards or labeled biomolecules facilitate native biomarker identification, for example in automate, high throughput data acquisition. A number of reference molecules are consistent with the disclosure herein.

Reference biomarker molecules are optionally isotopically labeled, such as using at least one of H2, H3, heavy nitrogen, heavy carbon, heavy oxygen, S35, P33, P32, and isotopic selenium. Alternately or in combination, reference biomarker molecules are chemically modified, such as using at least one of oxidized, acetylated, de-acetylated, methylated, and phosphorylated or otherwise modified to produce a slight but measurable change in overall mass. Alternately or in combination, reference biomarker molecules are nonhuman homologs of human proteins in the biomarker set.

A characteristic common to reference biomarkers include a repeatable offset co-migration with the native biomarker, such that the reference biomarker migrates near but not exactly with the biomarker of interest. Thus, detection of the biomarker is indicative that the native marker should be present at a predictable offset from the labeled biomarker.

A second characteristic common to some biomarker references is that they are readily identifiable in mass spectrometric data output. Often, biomarkers are identified in mass spectrometric output because their mass and therefore their position are precisely known in mass spectrometric output. By calculating their expected position and looking for a spot at that position having an expected concentration or signal, one can identify labeled markers in mass spectrometric output.

Mass-based identification of marker polypeptides is optionally further facilitated using any one or more of the following approaches. Firstly, an identified marker or marker set is run on its own, in the absence of a sample, so as to identify experimentally the exact positions where the markers run for a given mass spectrometric analysis. The markers are then run with the sample, and results are compared so as to identify the marker positions. This is done, for example, by overlaying results of one run involving only marker polypeptides with results of a second run comprising both marker polypeptides and sample biomarkers.

Secondly, various aliquots of the sample are provided with different concentrations of marker polypeptides. Mass spectrometric data for each of the marker dilution concentration variants are analyzed. Sample spots are expected (and observed) to show a high repeatability in spot location and intensity. Marker polypeptides, in contrast, show a high repeatability in spot location but a predictable variation in spot intensity that correlates with the concentration of marker added.

Thirdly, marker polypeptides are identified by their location on mass spectrometric outputs, and their identity is confirmed by the detection of a corresponding native protein or polypeptide at a predicted offset position, such that they indicate the presence of their native marker not by an independent signal but by presence as a ‘doublet’ having a predicted offset in a mass spectrometric output. This approach relies upon the native protein or polypeptide being present in the sample, but as this is often the case, the approach is valuable for the majority of the markers.

These approaches are not mutually exclusive. For example, one may generate a mass spectrometric output that only includes markers, and overlay that result against multiple sample mass spectrometric analyses having varying marker concentrations so as to identify markers at the expected locations and exhibiting the expected variation in spot signal strength relative to other runs. Independently or in combination with either of the approaches, one searches the mass spectrometric data to identify native spots at the expected offset from putative marker spots, thereby coming to finalized marker spot calls.

Alternately, identification is accomplished by heavy isotope radiolabeling. Such reference biomarkers are labeled consistent with mass spectrometric visualization, but are independently detectable through radiometric approaches, so as to facilitate their detection independent of the detection signal for native biomarkers in the sample.

Heavy isotope labeling, is particularly useful because it provides a predictable size-offset to facilitate native spot identification. However, other reference molecule labeling approaches are consistent with the disclosure herein.

Most often, a protein that yields a biomarker of interest is identified, and a reference biomarker is generated therefrom. Such protein biomarker reference molecules are, for example, synthesized with a detectable isotope of hydrogen, carbon, nitrogen, oxygen, sulfur or in some cases phosphate or even selenium. Reference biomarkers that are generated from synthetic versions of biomarkers of interest are beneficial because, aside from the mass offset, they are expected to behave comparably to native proteins in mass spectrometric analysis.

Alternately, non-protein biomarkers are used in some cases. Non-protein biomarkers have the advantage of often being simpler to synthesize. Additionally, one does not need the identity of the biomarker of interest to develop a non-protein biomarker. Rather, any labeled non-protein biomarker that migrates repeatably with a predictable offset from a biomarker of interest is consistent with the disclosure herein.

Aside from their role in marking or facilitating identification of native polypeptides, labeled reference markers are also useful in relative quantification of identified polypeptide spots on a mass spectrometric output. Labeled reference markers are introduced to a sample at known concentrations, and their signals in the mass spectrometric output are indicative of these concentrations. Spots corresponding to native proteins in the mass spectrometric output are readily and accurately quantified by comparing mass spectrometric signal strength to reference polypeptides of known concentration.

In some cases, two, more than two, up to 10%, 20%, 30%, 40%, 50%, 75%, 90%, up to all labeled reference markers are added at a single concentration, facilitating assessment of signal variation across polypeptide sizes and positions in the mass spectrometric output. Alternately or in combination, marker proteins or polypeptides are introduced at varying concentrations, such that one can compare a native mass spectrometric spot to a plurality of marker spots at varying intensities, thereby more accurately correlating a native spot signal to a reference signal of known concentration or amount. In some cases, various sets of marker proteins are introduced at a first concentration, while various other sets are introduced at other concentrations, thereby accomplishing both of the above-mentioned benefits. That is, markers at a common concentration or amount facilitate identification of variation in signal among markers and native mass spectrometric spots, while markers at a varying concentrations or amounts allow one to match native mass spectrometric spots to a spot of known amount or concentration across a broad range of amounts or concentrations, thereby providing an accurate reference for quantification of native mass spectrometric spots, and ultimately of native marker proteins or polypeptides, in a sample.

### Assessing Biomarker Signals

Biomarkers, either individual or collective biomarkers assembled into panels of at least two biomarkers, are assessed as to their significance as to patient health. A number of panel assessment approaches are consistent with the disclosure herein. Furthermore, additional approaches not explicitly recited herein are nonetheless consistent with the disclosure herein and there incorporation into a method or system is not inconsistent with the method of system falling within the scope of claims issuing from this disclosure.

Biomarker panel levels are obtained and assessed through at least one of the following approaches in various embodiments disclosed herein. In relatively simple cases, biomarker panel levels are compared to a reference level measured from an individual of known condition, and the patient is determined to share the condition if the biomarker levels do not differ significantly from the reference. Statistical assessment of whether or not two panels ‘differ significantly’ is made through any number of well-known or innovative approaches.

A number of methods of determining if one set of values differs significantly from another set of values are available. Such statistical tests (e.g., Analysis of Variation (ANOVA), t-tests, and Chi-squared analyses) are routine and have been for some time in the field of biological statistical analysis. Alternately, panel levels are evaluated using more elaborate computational approaches, such as machine learning or neural networking approaches.

Such tests, or other statistical tests known to one of skill in the art, are sufficient for assessing whether an increase, decrease, equal amount, numerical expression of standard deviations or some other protocol differs from a control reference set of values so as to warrant the classification of a measured set of panel values as differing substantially from a control set.

A person of ordinary skill in the art understands that they are directed to performing an appropriate statistical test to determine whether a measures set of values differs significantly from one or more reference sets of values.

For example, a person of ordinary skill in the art may wish to compare the accumulation levels of the proteins in a protein panel to a standard range derived from a plurality of reference samples. In such a situation, a person of ordinary skill in the art recognizes that a z-statistic or a t-statistic, for example, is an appropriate metric. A z-statistic makes use of the known reference population mean and variance to determine the probability that a sample drawn from the reference population would exhibit a more extreme measurement than a given cut-off. Cut-off values are determined such that a measurement more extreme than the cut off has a low probability (i.e., p-value) of being chosen from the reference population.

Furthermore, a person of ordinary skill in the art understands that a determination of statistically significant difference can be made using, for example, a t-test to determine the probability that their measurements could be provided by a reference sample. A person of ordinary skill in the art further recognizes that assessing the p-value cut-off depends on the application of the test results. Certain results, at a medical practitioner's or other user's discretion, may warrant more stringent evaluation of ‘significant’ that would otherwise be necessary.

For example, if the purpose of the test is to determine which patients receive a non-invasive, low-risk follow-up procedure, a relatively high p-value cut-off (e.g. p-value<0.1) might be select as a relatively high number of false positives will have little consequence. On the other hand, if the application of the test is a surgical or chemotherapeutic intervention, a much more stringent cutoff will likely be required to ensure a higher specificity. These considerations are well-understood and routine in the fields of epidemiology and medical test design.

Alternately or in combination, panel measurements are evaluated as to whether they pass a threshold at which a health status assessment is expected to change. That is, rather than, or in addition to scoring deviation from a reference panel value set or range, one assesses whether panel values, individually or collectively, surpass a threshold so as to constitute a change in health status assessment. In some cases the threshold is a sharp distinguisher between health status categories. Alternately, in some cases panels near a threshold are ‘not called,’ so that they are not categorized with confidence in either health category. Such a categorization strategy increases the confidence of categorization calls that are made, but leaves some panels uncategorized.

Alternately or in combination, samples are scored not by a binary yes/no categorization, but are assigned a percentile value relative to the reference database. The percentile value indicates, for example, where the sample measurements fit along a linear scale of the measurements or values of the database, such that one may determine from the analysis whether the sample values are typical of the reference dataset, or are outliers.

A number of approaches are available for fitting reference values on a linear scale relative to one another, and assigning a percentile value to a sample relative to the reference value. For example, reference values may be assessed on a marker by marker basis to determine mean or median values, and then sorted on a marker by marker basis as to how greatly the differ from the mean or median values. Rankings on a marker by marker bases are then assessed, for example, averaged or assigned statistical assessments of deviation from a mean or median value set (standard deviation determination, Chi-squared analysis, ANOVA, and other analyses are consistent with this approach), to determine which sample marker sets or panels, on a by marker basis or in aggregate, differ most substantially from the mean or median values per marker or in aggregate. A similar analysis is performed in a sample to be categorized, so as to assess the sample relative to the reference database. A number of alternative approaches to sample panel categorization are known in the art and consistent with the disclosure herein.

Similarly, a broad range of reference sets are consistent with the disclosure herein. As discussed above, some reference sets involve a single measurement, such as a single measurement of panel values from a single individual taken at a single time point. Such a measurement is optionally taken from a reference individual of known health status for the condition or status assessed by the panel, such that a substantially similar panel set indicates a common condition status. The reference individual is optionally a healthy individual or an individual suffering from a condition assayed by a panel, and may have any of a number of varying levels of severity of the condition. In some cases the reference panel is taken from the individual whose health is being assessed, but was obtained when a certain health condition was known (or later verified through ongoing health monitoring), so that difference from the level indicates a change in the individual.

Reference sets comprising more than one set of panel measurements are also consistent with the disclosure herein. Reference sets are generated from a plurality of individuals, such as 2, 3, 4, 5, 6, 7, 8, 9, 10, 20, 50, 100, 200, 500, 1000, 2000, 5000, 10,000, or more than 10,000 individuals, or a number comparable to a number listed herein. Preferably, the individuals share a common health status, and may in some cases be further sorted by a level of severity if their health status is positive for a condition having varying levels of severity. Alternately or in combination, reference sets are derived from multiple samples taken over time from at least one individual, such as the individual for which a later health assessment is to be made. Also contemplated are ‘two-dimensional’ reference sets, comprising panel information obtained from at least two individuals from at least two time points for some or all of the individuals.

When references comprise multiple panel sets, the references variously represent ranges of panel levels and panel constituent levels consistent with the health status of the reference. Thus, by using a multi-measurement panel, one is able to determine ranges of values consistent with a given health status, so as to assess whether an individual's panel levels fall within said ranges, do not differ significantly from said ranges, or do differ significantly from said ranges, so as to assess whether the individual warrants categorization as having the health status. Drawing from multiple panels provides for a representation of variation within panel levels consistent with a health categorization. Accordingly, one of skill in the art may tailor assessment statistical stringency to panel reference, such that assessment against references comprising multiple panels are given a higher degree of confidence at a given level of variation, relative to the same variation between a measured panel and a reference constructed from a single set of panel data.

Health conditions for which a reference set are developed include diseases as conventionally contemplated, such as various cancers, renal health, cardiovascular health, brain health, neuromuscular health or presence of a communicable disease. Alternately, more generalized ‘conditions’ are assessed by comparison to a reference, such as age, energy level, alertness, or other status. In such cases, an individual is assessed as to whether the individual presents a panel level consistent with the individual's chronological age, or whether the individual possesses panel information consistent with references of another age group.

### Machine Learning

Some embodiments involve machine learning as a component of database analysis, and accordingly some computer systems are configured to comprise a module having a machine learning capacity. Machine learning modules comprise at least one of the following listed modalities, so as to constitute a machine learning functionality.

Modalities that constitute machine learning variously demonstrate a data filtering capacity, so as to be able to perform automated mass spectrometric data spot detection and calling. This modality is in some cases facilitated by the presence of marker polypeptides, such as heavy isotope labeled polypeptides or other markers in a mass spectrometric analysis output, so that native peptides are readily identified and in some cases quantified. The markers are optionally added to samples prior to proteolytic digestion or subsequent to proteolytic digestion. Markers are in some embodiments present on a solid backing onto which a blood spot or other sample is deposited for storage or transfer prior to analysis via mass spectroscopy.

Modalities that constitute machine learning variously demonstrate a data treatment or data processing capacity, so as to render called data spots in a form conducive to downstream analysis. Examples of data treatment include but are not necessarily limited to log transformation, assigning of scaling ratios, or mapping data to crafted features so as to render the data in a form that is conducive to downstream analysis.

Machine learning data analysis components as disclosed herein regularly process a wide range of features in a mass spectrometric data set, such as 1 to 10,000 features, or 2 to 300,000 features, or a number of features within either of these ranges or higher than either of these ranges. In some cases, data analysis involves at least 1k, 2k, 3k, 4k, 5k, 6k, 7k, 8k, 9k, 10k, 20k, 30k, 40k, 50k, 60k, 70k, 80k, 90k, 100k, 120k, 140k, 160k, 180k, 200k, 220k, 2240k, 260k, 280k, 300k, or more than 300k features.

Features are selected using any number of approaches consistent with the disclosure herein. In some cases, feature selection comprises elastic net, information gain, random forest imputing or other feature selection approaches consistent with the disclosure herein and familiar to one of skill in the art.

Selected feature are assembled into classifiers, again using any number of approaches consistent with the disclosure herein. In some cases, classifier generation comprises logistic regression, SVM, random forest, KNN, or other classifier approaches consistent with the disclosure herein and familiar to one of skill in the art.

Machine learning approaches variously comprise implementation of at least one approach selected from the list consisting of ADTree, BFTree, ConjunctiveRule, DecisionStump, Filtered Classifier, J48, J48Graft, JRip, LADTree, NNge, OneR, OrdinalClassClassifier, PART, Ridor, SimpleCart, Random Forest and SVM.

Applying machine learning, or providing a machine learning module on a computer configured for the analyses disclosed herein, allows for the detection of relevant panels for asymptomatic disease detection or early detection as part of an ongoing monitoring procedure, so as to identify a disease or disorder either ahead of symptom development or while intervention is either more easily accomplished or more likely to bring about a successful outcome. Monitoring is often but not necessarily performed in combination with or in support of a genetic assessment indicating a genetic predisposition for a disorder for which a signature of onset or progression is monitored. Similarly, in some cases machine learning is used to facilitate monitoring of or assessment of treatment efficacy for a treatment regimen, such that the treatment regimen can be modified over time, continued or resolved as indicated by the ongoing proteomics mediated monitoring.

Machine learning approaches and computer systems having modules configured to execute machine learning algorithms facilitate identification of classifiers or panels in datasets of varying complexity. In some cases the classifiers or panels are identified from an untargeted database comprising a large amount of mass spectrometric data, such as data obtained from a single individual at multiple time points, samples taken from multiple individuals such as multiple individuals of a known status for a condition of interest or known eventual treatment outcome or response, or from multiple time points and multiple individuals.

Alternately, in some cases machine learning facilitates the refinement of a panel through the analysis of a database targeted to that panel, by for example collecting panel information for that panel from a single individual over multiple time points, when a health condition for the individual is known for the time points, or collecting panel information from multiple individuals of known status for a condition of interest, or collecting panel information from multiple individuals at multiple time points. As is readily apparent, in some cases collection of panel information is facilitated through the use of mass markers, such as heavy-labeled or ‘light-labeled’ mass markers that migrate so as to identify nearby unlabeled spots corresponding to the marked polypeptides. Thus, panel information is collected either alone or in combination with untargeted mass spectrometric data collection. Panel data is subjected to machine learning, for example on a computer system configured as disclosed herein, so as to identify a subset of panel markers that either alone or in combination with one or more non-panel markers analyzed through an untargeted approach, account for a health status signal. Thus, machine learning in some cases facilitates identification of a panel that is individually informative of a health status in an individual.

### Dried Blood Spot Analysis

Methods, databases and computers configured to receive mass spectrometric data as disclosed herein often involve processing mass spectrometric data sets that are spatially, temporally or spatially and temporally large. That is, datasets are generated that in some cases comprise large amounts of mass spectrometric data points per sample collected, are generated from from large numbers of collected samples, and are in some cases generated from multiple samples derived from a single individual.

Data collection is in some cases facilitated by depositing samples such as dried blood samples (or other readily obtained samples such as urine, sweat, saliva or other fluid or tissue) onto a solid framework such as a solid backing or solid three-dimensional framework. The sample such as a blood sample is deposited on the solid backing or framework, where it is actively or passively dried, facilitating storage or transport from a collection point to a location where it may be processed.

As disclosed herein, a number of approaches are available for recovering proteomic or other biomarker information from a dried sample such as a dried blood spot sample. In some cases samples are solubilized, for example in TFE, and subjected to proteolysis to generate fragments to be visualized by mass spectrometric analysis. Proteolysis is accomplished by enzymatic or non-enzymatic treatment. Exemplary proteases include trypsin, but also enzymes such as proteinase K, enteropeptidase, furin, liprotamase, bromelain, serratipeptidase, thermolysin, collagenase, plasmin, or any number of serine proteases, cysteine proteases or other specific or nonspecific enzymatic peptidases, used singly or in combination. Nonenzymatic protease treatments, such as high temperature, pH treatment, cyanogen bromide and other treatments are also consistent with some embodiments.

When particular mass spectrometric fragments are of interest or use in analysis, such as a biomarker panel indicative of a health condition status, it is often beneficial to include heavy-labeled or other markers as standard markers as described herein. Markers, as discussed, migrate on a mass spectrometric output at a known position and at a known offset relative to the sample fragments of interest. Inclusion of these markers often leads to ‘offset doublets’ in mass spectrometric output. By detecting these doublets, one can readily, either personally or through an automated data analysis workflow, identify particular spots of interest to a health condition status among and in addition to the full range of mass spectrometric output data. When the markers have known mass and amount, and optionally when the amount loaded into a sample varies among markers, the markers are also useful as mass standards, facilitating quantification of both the marker-associated fragments and the remaining fragments in the mass spectrometric output.

Standard markers are introduced to a sample either at collection, during or subsequent to resolubilization, prior to digestion or subsequent to digestion. That is, in some cases a sample collection structure such as a solid backing or a three-dimensional volume is ‘pre-loaded’ so as to have a standard marker or standard markers present prior to sample collection. Alternately, the standard markers are added to the collection structure subsequent to sample collection, subsequent to sample drying on the structure, during or subsequent to sample collection, during or subsequent to sample resolubilization, or during or subsequent to sample proteolysis treatment. In preferred embodiments, exactly or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, 150, 160, 170, 180, 190, 200, 225, 250, 275, 300, or more than 300 standard markers are added to a collection structure prior to sample collection, such that standard processing of the sample results in a mass spectrometric output having the standard markers included in the output without any additional processing of the sample. Accordingly, some methods disclosed herein comprise providing a collection device having sample markers introduced onto the surface prior to sample collection, and some devices or computer systems are configured to receive mass spectrometric data having standard markers included therein, and optionally to identify the mass spectrometric markers and their corresponding native mass fragment.

### Certain Definitions

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

“About” a number, as used herein, refers to a range including that number and spanning that number plus or minus 10% of that number. “About” a range refers to the range extended to 10% less than the lower limit and 10% greater than the upper limit of the range.

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, AmazonFire®, and Samsung® HomeSync®. Those of skill in the art will also recognize that suitable video game console operating systems include, by way of non-limiting examples, Sony® PS3, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-in

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon Kindle Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of biomarker information. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### Numbered Embodiments

Further understanding of the present disclosure is gained through review of the numbered embodiments recited herein. 1. A method of mass spectrometric output data processing comprising: generating a quantified output of the mass spectrometric output comparing the quantified output to a reference; and categorizing the quantified output relative to the reference, wherein practice of the method does not require human supervision. 2. The method of embodiment 1, or any of the above embodiments, wherein a second mass spectrometric output is received concurrently with said generating a quantified output of the mass spectrometric output of a first reference. 3. The method of embodiment 1, or any of the above embodiments, wherein the method is completed in no more than 8 hours. 4. The method of embodiment 1, or any of the above embodiments, wherein the method is completed in no more than 4 hours. 5. The method of embodiment 1, or any of the above embodiments, wherein the method is completed in no more than 2 hours. 6. The method of embodiment 1, or any of the above embodiments, wherein the method is completed in no more than 1 hour. 7. The method of embodiment 1, or any of the above embodiments, wherein the method is completed in no more than 30 minutes. 8. The method of embodiment 1, or any of the above embodiments, wherein the method is completed in no more than 5 minutes. 9. The method of embodiment 1, or any of the above embodiments, wherein the method is completed in no more than 1 minute. 10. The method of embodiment 1, or any of the above embodiments, comprising obtaining a fluid sample, and subjecting the fluid sample to mass spectrometric analysis, thereby generating a quantified output of the mass spectrometric analysis. 11. The method of embodiment 10, or any of the above embodiments, wherein the fluid sample is a dried fluid sample. 12. The method of embodiment 11, or any of the above embodiments, wherein obtaining the dried fluid sample comprises depositing a sample onto a sample collection backing. 13. The method of embodiment 10, or any of the above embodiments, wherein separating plasma from whole blood on the backing comprises contacting whole blood to a filter on the backing. 14. The method of embodiment 1, or any of the above embodiments, wherein subjecting the dried fluid sample to mass spectrometric analysis comprises volatilizing the sample. 15. The method of embodiment 11, or any of the above embodiments, wherein subjecting the dried fluid sample to mass spectrometric analysis comprises subjecting the sample to proteolytic degradation. 16. The method of embodiment 15, or any of the above embodiments, wherein the proteolytic degradation comprises enzymatic degradation. 17. The method of embodiment 16, or any of the above embodiments, wherein the enzymatic degradation comprises contacting a sample to at least one of ArgC, AspN, chymotrypsin, GluC, LysC, LysN, trypsin, snake venom diesterase, pectinase, papain, alcanase, neutrase, snailase, cellulase, amylase, and chitinase. 18. The method of embodiment 16, or any of the above embodiments, wherein the enzymatic degradation comprises trypsin degradation. 19. The method of embodiment 15, or any of the above embodiments, wherein the proteolytic degradation comprises nonenzymatic degradation. 20. The method of embodiment 19, or any of the above embodiments, wherein the nonenzymatic degradation comprises at least one of heat, acidic treatment, and salt treatment. 21. The method of embodiment 19, or any of the above embodiments, wherein the nonenzymatic degradation comprises contacting a sample to at least one of hydrochloric acid, formic acid, acetic acid, hydroxide bases, cyanogen bromide, 2-nitro-5-thiocyanobenzoate, and hydroxylamine. 22. The method of any one of embodiments 1-21, wherein generating a quantified output of the mass spectrometric analysis comprises quantifying at least 20 mass points. 23. The method of any one of embodiments 1-21, wherein generating a quantified output of the mass spectrometric analysis comprises quantifying at least 50 mass points. 24. The method of any one of embodiments 1-21, wherein generating a quantified output of the mass spectrometric analysis comprises quantifying at least 100 mass points. 25. The method of any one of embodiments 1-21, wherein generating a quantified output of the mass spectrometric analysis comprises quantifying at least 5,000 mass points. 26. The method of any one of embodiments 1-21, wherein generating a quantified output of the mass spectrometric analysis comprises quantifying at least 15,000 mass points. 27. The method of any one of embodiments 1-21, wherein generating a quantified output of the mass spectrometric analysis is completed in no more than 30 minutes. 28. The method of any one of embodiments 1-21, wherein generating a quantified output of the mass spectrometric analysis is completed in no more than 15 minutes. 29. The method of any one of embodiments 1-21, wherein generating a quantified output of the mass spectrometric analysis is completed in no more than 10 minutes. 30. The method of any one of embodiments 1-21, wherein generating a quantified output of the mass spectrometric analysis is completed in no more than 5 minutes. 31. The method of any one of embodiments 1-21, wherein generating a quantified output of the mass spectrometric analysis is completed in no more than 1 minute. 32. The method of any one of embodiments 1-21, wherein generating a quantified output of the mass spectrometric analysis is automated. 33. The method of any one of embodiments 1-21, wherein generating a quantified output of the mass spectrometric analysis comprises generating an adjusted abundance value. 34. The method of any one of embodiments 1-21, wherein generating a quantified output of the mass spectrometric analysis comprises generating an adjusted mz value. 35. The method of any one of embodiments 1-21, wherein generating a quantified output of the mass spectrometric analysis comprises performing a convolution operation to reduce pixel-by-pixel noise of the mass spectrometry data; and identifying a plurality of features of the sample, wherein identifying the plurality of features comprises identifying a plurality of peaks of the mass spectrometry data, and determining a respective mz value and a respective LC value for the plurality of peaks. 36. The method of any one of embodiments 1-21, wherein generating a quantified output of the mass spectrometric analysis comprises receiving data for a plurality of identified peaks from mass spectrometry data of the sample; filtering the plurality of identified peaks to provide a filtered set of peaks, the filtering comprising (1) a first filtering process on the data for the plurality of identified peaks, the first filtering process comprising a peak contrast filtering process, and (2) a second filtering process for removing at least one of a spurious peak and a peak corresponding a calibrant analyte; and selecting a subset of peaks from the plurality of peaks, the subset of peaks comprising peaks corresponding to molecular feature isotopic clusters. 37. The method of any one of embodiments 1-21, wherein generating a quantified output of the mass spectrometric analysis comprises receiving mass spectrometry data of the sample, the mass spectrometry data comprising data for a peptide; and determining a metric value indicative of a likelihood of successful sequencing of the peptide. 38. The method of any one of embodiments 1-21, wherein generating a quantified output of the mass spectrometric analysis comprises receiving mass spectrometry data of the sample, the mass spectrometry data comprising a molecular mass value of the sample; and determining, using the mass defect histogram library, a mass defect probability for identifying the molecular mass value, wherein the mass defect probability is indicative of a probability the molecular mass value corresponds to a peptide from the sample. 39. The method of any one of embodiments 1-21, wherein generating a quantified output of the mass spectrometric analysis comprises receiving tandem mass spectrometry data of the sample, the tandem mass spectrometry data comprising respective molecular mass values for a plurality of identified peaks; and determining a metric value indicative of correspondence between the molecular mass values and molecular mass values of known peptides fragments. 40. The method of any one of embodiments 1-21, wherein generating a quantified output of the mass spectrometric analysis comprises receiving tandem mass spectrometry data of the sample, the tandem mass spectrometry data comprising respective molecular mass values for a plurality of identified peaks; and determining a metric value indicative of correspondence between the molecular mass values and molecular mass values of known peptides. 41. The method of any one of embodiments 1-21, wherein generating a quantified output of the mass spectrometric analysis comprises identifying data features corresponding to the set of targeted mass spectrometric features; determining characteristics comprising mass, charge and elution time for the data features; and calculating deviation between targeted mass spectrometric feature characteristics and data feature characteristic. 42. The method of any one of embodiments 1-21, wherein generating a quantified output of the mass spectrometric analysis comprises comparing mass spectrometry data to the set of protein modifications and digestion variants; and assessing the frequency of at least one of protein modifications and digestion frequency. 43. The method of any one of embodiments 1-21, wherein generating a quantified output of the mass spectrometric analysis comprises identifying test peptide signals in a mass spectrometric output. 44. The method of any one of embodiments 1-21, wherein generating a quantified output of the mass spectrometric analysis comprises identifying reference clusters having exactly one feature per sample; assigning an index area derived from the reference clusters; and mapping nonreference clusters onto the index area. 45. The method of any one of embodiments 1-21, wherein generating a quantified output of the mass spectrometric analysis comprises identifying features having common m/z ratios across a plurality of samples; aligning said features across a plurality of samples; bringing LC times for said features in line; and clustering said features. 46. The method of any one of embodiments 1-21, wherein generating a quantified output of the mass spectrometric analysis comprises identifying features having common m/z ratios and common LC times across a plurality of fractions of a sample; assigning to a common cluster features sharing a common m/z ratios and common LC times in adjacent fractions; and discarding said cluster and retain said features when said cluster has at least one of a size above a threshold and an LC time above a threshold. 47. The method of any one of embodiments 1-21, wherein generating a quantified output of the mass spectrometric analysis comprises choosing a first random subset of fraction outputs; counting the number of unique pieces of information for the first random subset of fraction outputs; choosing a second random subset of fraction outputs; counting the number of unique pieces of information for the second random subset of fraction outputs; and selecting the random subset of fraction outputs having the greater number of unique pieces of information. 48. The method of any one of embodiments 1-21, wherein generating a quantified output of the mass spectrometric analysis comprises identifying measured features for said mass spectrometric fraction outputs; calculating average m/z and LC time values for measured features appearing in multiple mass spectrometric fraction outputs; assaying for unidentified features sharing at least one of average m/z and LC time values with said measured features; and assigning at least one of said unidentified features to a cluster of a measured feature, so as to generate at least one inferred mass feature. 49. The method of any one of embodiments 1-21, wherein generating a quantified output of the mass spectrometric analysis comprises calculating expected LC retention times; calculating standard deviation values of expected LC retention times; comparing expected LC retention times to observed associated LC retention times; and discarding mass spectrometric peptide identification calls for which expected LC retention times differ from observed associated LC retention times by more than standard deviation values. 50. The method of any one of embodiments 1-21, wherein generating a quantified output of the mass spectrometric analysis comprises identifying features corresponding to common peptides and having differing LC retention times in the plurality of mass spectrometry outputs; applying an LC retention time shift to one of the mass spectrometry outputs so as to bring the differing LC times into closer alignment for the features corresponding to common peptides; applying the LC retention time shift to additional features in proximity to the features corresponding to common peptides in the mass spectrometry output; and discarding mass spectrometric peptide identification calls for which expected LC retention times differ from observed associated LC retention times by more than standard deviation values. 51. The method of any one of embodiments 1-21, wherein generating a quantified output of the mass spectrometric analysis comprises grouping proteins sharing at least one common peptide; determining a minimum number of proteins per group; and determining a sum for the minimum number of proteins per group for all groups. 52. The method of any one of embodiments 1-21, wherein generating a quantified output of the mass spectrometric analysis comprises constructing a command line in a format compatible with a given search engine; initiating execution of the search engine; parsing the search engine output; and configuring the output into a standard format. 53. The method of any one of embodiments 1-21, wherein generating a quantified output of the mass spectrometric analysis comprises parsing file contents from a memory unit into key-value pairs; read each key-value pair into a standard format; and writing the standard format key-value pairs into an output file. 54. The method of any one of embodiments 1-21, wherein generating a quantified output of the mass spectrometric analysis comprises parsing a file into an array of key-value pairs representative of tandem mass spectra and corresponding attributes; obtaining corresponding precursor ion attributes; replacing mass spectrum file values using precursor ion attributes when precursor ion attributes are indicated to be accurate; and configuring the file into a flat format output. 55. The method of any one of embodiments 1-21, wherein generating a quantified output of the mass spectrometric analysis comprises receiving a mass spectrometry output having a plurality of unidentified features; including features having a z-value of greater than 1 up to and including 5; clustering included features by retention time to form clusters; de-prioritizing clusters for which verification has been previously performed; selecting a single feature per cluster; and verifying a feature of at least one cluster. 56. The method of any one of embodiments 1-21, wherein generating a quantified output of the mass spectrometric analysis comprises generating a processed dataset from one of a plurality of received mass spectrometric output; and incorporating the processed dataset into a processed study dataset. 57. The method of any one of embodiments 1-21, wherein generating a quantified output of the mass spectrometric analysis comprises receiving a first mass spectrometric output and a second mass spectrometric output; performing a quality analysis on the first mass spectrometric output; incorporating the first mass spectrometric output into a processed dataset; performing a quality analysis on the second mass spectrometric output; incorporating the second mass spectrometric output into the processed dataset; wherein performing the quality analysis on the first mass spectrometric output and receiving the second mass spectrometric output are concurrent. 58. The method of any one of embodiments 1-21, wherein generating a quantified output of the mass spectrometric analysis does not comprise human analysis of the mass spectrometric analysis. 59. The method of any one of embodiments 1-21, wherein generating a quantified output of the mass spectrometric analysis comprises identifying at least 3 reference mass outputs in the mass spectrometric analysis. 60. The method of any one of embodiments 1-21, wherein generating a quantified output of the mass spectrometric analysis comprises identifying at least 6 reference mass outputs in the mass spectrometric analysis. 61. The method of any one of embodiments 1-21, wherein generating a quantified output of the mass spectrometric analysis comprises identifying at least 10 reference mass outputs in the mass spectrometric analysis. 62. The method of any one of embodiments 1-21, wherein generating a quantified output of the mass spectrometric analysis comprises identifying at least 100 reference mass outputs in the mass spectrometric analysis. 63. The method of embodiment 59, or any of the above embodiments, wherein the at least 3 reference mass outputs are introduced to the sample prior to analysis. 64. The method of embodiment 59, or any of the above embodiments, wherein the at least 3 reference mass outputs differ from sample mass outputs by known amounts. 65. The method of embodiment 59, or any of the above embodiments, wherein the at least 3 reference mass outputs have known amounts. 66. The method of embodiment 65, or any of the above embodiments, comprising comparing reference mass output amounts to sample output amounts. 67. The method of embodiment 1, or any of the above embodiments, wherein comparing the quantified output to a reference comprises identifying a subset of the sample mass output, and comparing said subset of the sample mass output to the reference. 68. The method of embodiment 1, or any of the above embodiments, wherein the reference comprises at least one sample output of known status for a health category. 69. The method of embodiment 1, or any of the above embodiments, wherein the reference comprises at least ten sample outputs of known status for a health category. 70. The method of embodiment 1, or any of the above embodiments, wherein the reference comprises at least ten samples of unknown health status for a health category. 71. The method of embodiment 1, or any of the above embodiments, wherein the reference comprises predicted values for a health status for a health category. 72. The method of embodiment 1, or any of the above embodiments, wherein the reference comprises samples taken from at least two individuals. 73. The method of embodiment 1, or any of the above embodiments, wherein the reference comprises samples taken from at least two time points. 74. The method of embodiment 1, or any of the above embodiments, wherein the reference comprises a sample taken from a source common to the sample. 75. The method of embodiment 1, or any of the above embodiments, wherein categorizing the quantified output relative to the reference comprises assigning a health category status to an individual source of the sample. 76. The method of embodiment 1, or any of the above embodiments, wherein categorizing the quantified output relative to the reference comprises assigning the reference health category status to an individual source of the sample. 77. The method of embodiment 1, or any of the above embodiments, wherein categorizing the quantified output relative to the reference comprises assigning the reference health category status to an individual source of the sample. 78. The method of embodiment 1, or any of the above embodiments, wherein categorizing the quantified output relative to the reference comprises assigning a percentage value to an individual source of the sample. 79. The method of embodiment 78, or any of the above embodiments, wherein the percentage value represents the position of the sample relative to the reference. 80. A method comprising: obtaining a biological sample subjecting the biological sample to mass spectrometric analysis; generating a quantified output of the mass spectrometric analysis; comparing the quantified output to a reference; and categorizing the quantified output relative to the reference, wherein the method does not comprise human supervision. 81. A method comprising: obtaining a biological sample; subjecting the biological sample to mass spectrometric analysis; generating a quantified output of the mass spectrometric analysis; comparing the quantified output to a reference; and categorizing the quantified output relative to the reference, wherein the method is automated. 82. A method comprising: obtaining a biological sample; subjecting the biological sample to mass spectrometric analysis; generating a quantified output of the mass spectrometric analysis; comparing the quantified output to a reference; and categorizing the quantified output relative to the reference, wherein the generating, comparing and categorizing are completed in no more than 30 minutes. 83. The method of embodiment 82, or any of the above embodiments, wherein the generating, comparing and categorizing are completed in no more than 15 minute. 84. The method of embodiment 82, or any of the above embodiments, wherein the generating, comparing and categorizing are completed in no more than 10 minutes. 85. The method of embodiment 82, or any of the above embodiments, wherein the generating, comparing and categorizing are completed in no more than 5 minutes. 86. The method of embodiment 82, or any of the above embodiments, wherein the generating, comparing and categorizing are completed in no more than 1 minute. 87. A computer system for mass spectrometry analysis of a sample, comprising: a processor; and a memory to store a computer program, the computer program comprising instructions for: receiving raw mass spectrometry data of the sample, the raw mass spectrometry data comprising corresponding abundance values and corresponding mz values for features contained in the sample; performing at least one of (1) generating an adjusted abundance value, and (2) generating an adjusted mz value; and generating a text based data file using the raw mass spectrometry data. 88. The system of embodiment 87, or any of the above embodiments, wherein the computer program further comprises instructions for: determining a plurality of abundance values from the raw mass spectrometry data; generating a corresponding adjusted abundance value from each abundance value of the plurality of abundance values, wherein generating the adjusted abundance value comprises setting an abundance value to zero if the abundance value is less than a predetermined abundance value threshold. 89. The system of embodiment 87, or any of the above embodiments, wherein the computer program further comprises instructions for: determining a plurality of mz values from the raw mass spectrometry data; generating a corresponding adjusted mz value from each mz value of the plurality of mz values, wherein generating the adjusted mz value comprises setting a mz value to a predetermined mz value. 90. The system of embodiment 87, or any of the above embodiments, wherein receiving the raw mass spectrometry data comprises receiving raw mass spectrometry data from one mass scan of a sample. 91. The system of embodiment 87, or any of the above embodiments, wherein receiving the raw mass spectrometry data comprises receiving raw mass spectrometry data from at least two mass scans of a sample. 92. The system of embodiment 87, or any of the above embodiments, wherein the computer program further comprises instructions for storing pairs of adjusted abundance values and adjusted mz values. 93. A computer system for mass spectrometry analysis of a sample, comprising: a processor; and a memory to store a computer program, the computer program comprising instructions for: receiving a text based mass spectrometry data of the sample, the text based mass spectrometry data comprising mass spectrometry data from a plurality of mass scans; and generating an image pixel representation of the mass spectrometry data for the plurality of mass scans, the image pixel representation comprising a plurality of pixels, wherein generating the image pixel representation comprises determining a value of each pixel of the plurality of pixels, and wherein determining the value of each pixel comprises accumulating abundance values across the plurality of scans for each pixel. 94. The system of embodiment 93, or any of the above embodiments, wherein the computer program further comprises instructions for mapping each mz value of the mass spectrometry data to a corresponding first value between 0 and 1.95. The system of embodiment 93, or any of the above embodiments, wherein the computer program further comprises instructions for mapping each LC value of the mass spectrometry data to a corresponding second value between 0 and 1.96. The system of embodiment 93, or any of the above embodiments, wherein generating the image pixel representation comprises generating the plurality of pixels comprising a width of W pixels and a height of H pixels. 97. The system of embodiment 93, or any of the above embodiments, wherein accumulating the abundances comprises performing an interpolation. 98. The system of embodiment 93, or any of the above embodiments, wherein accumulating the abundances comprises performing a linear interpolation. 99. The system of embodiment 93, or any of the above embodiments, wherein accumulating the abundances comprises performing a nonlinear interpolation. 100. The system of embodiment 97, or any of the above embodiments, wherein accumulating the abundances comprises performing an integration. 101. A computer system for mass spectrometry analysis of a sample, comprising: a processor; and a memory to store a computer program, the computer program comprising instructions for: receiving mass spectrometry data of the sample; performing a convolution operation to reduce pixel-by-pixel noise of the mass spectrometry data; and identifying a plurality of features of the sample, wherein identifying the plurality of features comprises identifying a plurality of peaks of the mass spectrometry data, and determining a respective mz value and a respective LC value for the plurality of peaks. 102. The system of embodiment 101, or any of the above embodiments, wherein identifying the plurality of features comprises determining a respective peak_height and a respective peak area for the plurality of peaks. 103. The system of embodiment 101, or any of the above embodiments, wherein identifying the plurality of features comprises subjecting the mass spectrometry data to a machine learning analysis. 104. The system of embodiment 101, or any of the above embodiments, wherein identifying the plurality of features comprises subjecting the mass spectrometry data to an artificial intelligence analysis. 105. The system of embodiment 101, or any of the above embodiments, wherein identifying the plurality of peaks comprises selecting a peak comprising a height than a predetermined threshold, and greater than corresponding heights of at least eight adjacent peaks. 106. A computer system configured for mass spectrometry analysis of a sample, comprising: a processor; and a memory to store a computer program, the computer program comprising instructions for: receiving data for a plurality of identified peaks from mass spectrometry data of the sample; filtering the plurality of identified peaks to provide a filtered set of peaks, the filtering comprising (1) a first filtering process on the data for the plurality of identified peaks, the first filtering process comprising a peak contrast filtering process, and (2) a second filtering process for removing at least one of a spurious peak and a peak corresponding a calibrant analyte; and selecting a subset of peaks from the plurality of peaks, the subset of peaks comprising peaks corresponding to molecular feature isotopic clusters. 107. The system of embodiment 106, or any of the above embodiments, wherein the data for the plurality of identified peaks comprises a respective mz value, a respective LC value, a respective abundance value, and a respective chromatographic value for each of the plurality of identified peaks. 108. The system of embodiment 107, or any of the above embodiments, wherein the respective chromatographic value for the plurality of identified peak comprises a peak width value. 109. The system of embodiment 106, or any of the above embodiments, wherein selecting the subset of peaks comprises providing a respective mz value, a respective LC value, a respective peak height value, a respective peak area value, and a respective chromatographic value for each of the subset of peaks. 110. The system of embodiment 106, or any of the above embodiments, wherein the computer program further comprises instructions for calibrating each of the plurality of filtered peaks to provide a plurality of calibrated peaks, the calibrating comprising calibrating respective mz values for each of the plurality of filtered peaks. 111. The system of embodiment 110, or any of the above embodiments, wherein the computer program further comprises instructions for generating a 2-dimensional matrix to bin the plurality of calibrated peaks to provide a plurality of binned peaks. 112. The system of embodiment 111, or any of the above embodiments, wherein the computer program further comprises instructions for combining the plurality of binned peaks to form the isotopic clusters. 113. The system of embodiment 106, or any of the above embodiments, wherein the computer program further comprises instructions to mapping the isotopic clusters to identified molecular features. 114. A computer system configured for mass spectrometry analysis of a sample, comprising: a processor; and a memory to store a computer program, the computer program comprising instructions for: receiving mass spectrometry data of the sample, the mass spectrometry data comprising data for a peptide; and determining a metric value indicative of a likelihood of successful sequence determination for the peptide. 115. The system of embodiment 114, or any of the above embodiments, wherein receiving the mass spectrometry data comprises receiving mass spectrometry data for an isotopic envelope of a feature, an estimated mz value corresponding to the feature and a charge state corresponding to the feature. 116. A computer system configured for mass spectrometry analysis of a sample, comprising: a processor; and a memory to store a computer program, the computer program comprising instructions for: providing a mass defect histogram library comprising a mass defect histogram for each of a plurality of neutral mass values; receiving mass spectrometry data of the sample, the mass spectrometry data comprising a molecular mass value of the sample; and determining, using the mass defect histogram library, a mass defect probability for identifying the molecular mass value, wherein the mass defect probability is indicative of a probability the molecular mass value corresponds to a peptide from the sample. 117. The system of embodiment 116, or any of the above embodiments, wherein the computer program further comprises instructions for identifying the peptide using the mass defect histogram library. 118. The system of embodiment 116, or any of the above embodiments, wherein providing the mass defect histogram library comprises generating the mass defect histogram library using predetermined neutral mass values. 119. The system of embodiment 116, or any of the above embodiments, wherein the computer program further comprises instructions for receiving a library comprising a plurality of neutral mass values corresponding to a plurality of known peptides. 120. The system of embodiment 119, or any of the above embodiments, wherein the computer program further comprises instructions for normalizing each of the plurality of neutral mass values corresponding to the plurality of known peptides. 121. The system of embodiment 116, or any of the above embodiments, wherein the computer program further comprises instructions for receiving a library comprising a plurality of neutral mass values corresponding to a plurality of predicted peptides. 122. The system of embodiment 119, or any of the above embodiments, wherein the computer program further comprises instructions for normalizing each of the plurality of neutral mass values corresponding to the plurality of predicted peptides. 123. A computer system configured for mass spectrometry analysis of a sample, comprising: a processor; and a memory to store a computer program, the computer program comprising instructions for: receiving tandem mass spectrometry data of the sample, the tandem mass spectrometry data comprising respective molecular mass values for a plurality of identified peaks; and determining a metric value indicative of correspondence between the molecular mass values and molecular mass values of known peptides fragments. 124. The system of embodiment 123, or any of the above embodiments, wherein receiving the tandem mass spectrometry data comprises receiving: (1) a mass probability value, (2) a mz value, and (3) a z value. 125. The system of embodiment 123, or any of the above embodiments, wherein the computer program further comprises instructions for: receiving a peptide mass value library comprising a plurality of mass peptide values; determining a neutral mass value; and determining a defect probability value. 126. The system of embodiment 125, or any of the above embodiments, wherein determining the defect probability value comprises interpolating the plurality of mass peptide values using the neutral mass value. 127. A computer system configured for mass spectrometry analysis of a sample, comprising: a processor; and a memory to store a computer program, the computer program comprising instructions for: receiving tandem mass spectrometry data of the sample, the tandem mass spectrometry data comprising respective molecular mass values for a plurality of identified peaks; and determining a metric value indicative of correspondence between the molecular mass values and molecular mass values of known peptides. 128. The system of embodiment 127, or any of the above embodiments, wherein receiving the tandem mass spectrometry data comprises receiving both a respective mz value and a respective abundance value for each of the plurality of identified peaks. 129. The system of embodiment 127, or any of the above embodiments, wherein determining the metric value comprises determining a weighted average. 130. The system of embodiment 129, or any of the above embodiments, wherein determining the weighted average comprises determining the weighted average based on respective abundance values for the plurality of identified peaks. 131. A computer system configured to identify mass spectrometry output feature characteristics, comprising: a memory unit configured to receive a set of targeted mass spectrometric features having characteristics comprising mass, charge and elution time; a computation unit configured to identify data features corresponding to the set of targeted mass spectrometric features; to determine characteristics comprising mass, charge and elution time for the data features; to calculate deviation between targeted mass spectrometric feature characteristics and data feature characteristic; an output unit configured to provide mass spectrometric information comprising at least one of neutral mass, charge state, observed elution time, and deviation. 132. The computer system of embodiment 131, or any of the above embodiments, wherein said characteristics comprise abundance. 133. The computer system of embodiment 131, or any of the above embodiments, wherein said characteristics comprise intensity. 134. A computer system configured to assess protein mass spectrometry input status, comprising: a memory unit configured to receive a set of protein modifications and digestion variants; a computation unit configured to compare mass spectrometry data to the set of protein modifications and digestion variants; and to assess the frequency of protein modifications; and an output unit configured to report an assessment of protein modifications. 135. A computer system configured to assess mass spectrometry apparatus performance, comprising: a memory unit configured to receive performance parameters for a set of test analyte signals; a computation unit configured to identify test analyte signals in a mass spectrometric output; and assess difference between said signals and said performance parameters; and an output unit configured to provide assessment of the difference between said signals and said performance parameters. 136. The computer system of embodiment 135, or any of the above embodiments, wherein the test peptides are selected from the list of peptides in table 3.137. The computer system of embodiment 135, or any of the above embodiments, wherein the analyte signals comprise peptide signals corresponding to test peptide accumulation levels. 138. The computer system of embodiment 135, or any of the above embodiments, wherein the analyte signals comprise poly-leucine peptide signals. 139. The computer system of embodiment 135, or any of the above embodiments, wherein the analyte signals comprise poly-glycine peptide signals. 140. The computer system of embodiment 135, or any of the above embodiments, wherein the apparatus performance is assessed as to at least one of mass accuracy, LC retention time, LC peak shape, and abundance measurement. 141. The computer system of embodiment 135, or any of the above embodiments, wherein the apparatus performance is assessed as to at least one of number of detected peptides, relative change in number of features, maximum abundance error, overall mean abundance shift; standard deviation in abundance shift; maximum m/z deviation; maximum peptide retention time; and maximum peptide chromatographic full-width half maximum. 142. A computer system configured to normalize mass spectrometric peak areas, comprising: a memory unit configured to receive a set of extracted mass spectrometry peak areas; a computation unit configured to identify reference clusters having exactly one feature per sample; to assign an index area derived from the reference clusters; and to map nonreference clusters onto the index area; and an output unit configured to provide corrected peak area outputs. 143. A computer system configured to identify common features of mass spectrometric output across a plurality of samples, comprising: a memory unit configured to receive a set of mass spectrometric outputs; a computation unit configured to identify features having common m/z ratios across a plurality of samples; to align said features across a plurality of samples; to bring LC times for said features in line; and to cluster said features; and an output unit configured to provide identification of at least one feature common to at least two members of the set of mass spectrometric outputs. 144. The computer system of embodiment 143, or any of the above embodiments, wherein being configured to align said features across a plurality of samples comprises being configured to apply a nonlinear retention time warping procedure. 145. A computer system configured to cluster peptide features appearing in a plurality of mass spectrometry fractions, comprising: a memory unit configured to receive a set of mass spectrometric outputs; a computation unit configured to identify features having common m/z ratios and common LC times across a plurality of fractions of a sample; to assign to a common cluster features sharing a common m/z ratios and common LC times in adjacent fractions; and to discard said cluster and retain said features when said cluster has at least one of a size above a threshold and an LC time above a threshold; and an output unit configured to provide cluster identification for a plurality of feature clusters. 146. The computer system of embodiment 145, or any of the above embodiments, wherein said size has a threshold of 75 ppm and said LC time of at least 50 seconds. 147. A computer system configured to rank mass spectrometry fractions according to information content, comprising: a memory unit configured to receive a set of mass spectrometric fraction outputs; a computation unit configured to choose a first random subset of fraction outputs; to count the number of unique pieces of information for the first random subset of fraction outputs; to choose a second random subset of fraction outputs; to count the number of unique pieces of information for the second random subset of fraction outputs; and to select the random subset of fraction outputs having the greater number of unique pieces of information; and an output unit configured to provide fraction subset information correlated to number of unique pieces of information. 148. A computer system configured to re-extract peptide features appearing in a mass spectrometry output, comprising: a memory unit configured to receive a set of mass spectrometric outputs and to store scoring information for measured features for said mass spectrometric fraction outputs; a computation unit configured to identify measured features for said mass spectrometric outputs; to calculate average m/z and LC time values for measured features appearing in multiple mass spectrometric outputs; to assay for unidentified features sharing at least one of average m/z and LC time values with said measured features; and assigning at least one of said unidentified features to a cluster of a measured feature, so as to generate at least one inferred mass feature; and an output unit configured to provide said measured features and said at least one inferred mass feature observations. 149. A computer system configured to filter inconsistent peptide identification calls, comprising: a memory unit configured to receive a set of mass spectrometric peptide identification calls and associated mass spectrometric LC retention times; a computation unit configured to calculate expected LC retention times; to calculate standard deviation values of expected LC retention times; to compare expected LC retention times to observed associated LC retention times; and to discard mass spectrometric peptide identification calls for which expected LC retention times differ from observed associated LC retention times by more than standard deviation values; and an output unit configured to provide filtered peptide identification calls. 150. A computer system configured to adjust retention times so as to align fragments sharing m/z ratios, comprising: a memory unit configured to receive a set of mass spectrometric peptide identification calls and associated mass spectrometric LC retention times for a plurality of mass spectrometry outputs; a computation unit configured to identify features corresponding to common peptides and having differing LC retention times in the plurality of mass spectrometry outputs; to apply an LC retention time shift to one of the mass spectrometry outputs so as to bring the differing LC times into closer alignment for the features corresponding to common peptides; to apply the LC retention time shift to additional features in proximity to the features corresponding to common peptides in the mass spectrometry output; and to discard mass spectrometric peptide identification calls for which expected LC retention times differ from observed associated LC retention times by more than standard deviation values; and an output unit configured to provide a retention time adjusted mass spectrometry output. 151. A computer system configured to calculate a minimum assignable protein count for a mass spectrometric output, the computer system comprising: a memory unit configured to receive a list of identified peptides in a mass spectroscopy output, and a mapping of said identified peptides to all proteins that contain said peptides; a computation unit configured to group proteins sharing at least one common peptide; to determine a minimum number of proteins per group; and to determine a sum for the minimum number of proteins per group for all groups; and an output unit configured to provide a minimum number of proteins consistent with the list of identified peptides. 152. A computer system configured to maintain uniform proteomic peptide assignment across peptide analysis platforms, the system comprising: a memory unit configured to receive proteomic peptide assignments in a standard format; and a computation unit configured to construct a command line in a format compatible with a given search engine; initiate execution of the search engine; parse the search engine output; and configure the output into a standard format. 153. The computer system of embodiment 152, or any of the above embodiments, wherein the computation unit is configured to run a relational database Object operation. 154. The computer system of embodiment 152, or any of the above embodiments, wherein the standard configuration comprises at least one parameter selected from a list consisting of precursor ion max mass error, fragment ion max mass error, rank, expectation value, score, processing threads, fasta database and post-translational modifications. 155. A computer system configured to extract tandem mass spectra and assign individual headers with specific spectrum information, comprising: a memory unit comprised to receive mass spectra information; a computation unit configured to parse file contents from the memory unit into key-value pairs; read each key-value pair into a standard format; and write the standard format key-value pairs into an output file. 156. The computer system of embodiment 155, or any of the above embodiments, wherein the key-value pairs comprise at least one of DATA_FILE, EXPERIMENT NO, LCMS_SCAN_NO, LCMS_LCTIME, OBSERVED_MZ, OBSERVED_Z, TANDEM_LCMS_MAX_ABUNDANCE, TANDEM_LCMS_PRECURSOR_ABUNDANCE, TANDEM_LCMS_SNR, and LCMS SCAN MGF NO. 157. A computer system configured to compute a tandem mass spectra correction, comprising: a memory unit configured to receive a proteomics mass spectrum file; and a computation unit configured to parse the file into an array of key-value pairs representative of tandem mass spectra and corresponding attributes; to obtain corresponding precursor ion attributes; to replace mass spectrum file values using precursor ion attributes when precursor ion attributes are indicated to be accurate; and configure the file into a flat format output. 158. A computer system configured to compute a false discovery rate for feature assignments, comprising: a memory unit configured to receive a list of proteomics search engine results comprising feature assignments; a computation unit configured to assess the list relative to randomly generated lists and assign key-valued pairs to the feature assignments; and an output unit configured to provide a measure of statistical confidence for the feature assignments. 159. The computer system of embodiment 158, or any of the above embodiments, wherein the computation unit is configured to compute an expectation value for a given false discovery rate using Benjamini-Hochberg-Yekutieli computation. 160. A method of mass spectrometry feature verification selection, comprising: receiving a mass spectrometry output having a plurality of unidentified features; including features having a z-value of greater than 1 up to and including 50; clustering included features by retention time to form clusters; de-prioritizing clusters for which verification has been previously performed; selecting a single feature per cluster; and verifying a feature of at least one cluster. 161. The method of embodiment 160, or any of the above embodiments, wherein a cluster having an identification score of greater than a lowest expected valid score is de-prioritized. 162. The method of embodiment 160, or any of the above embodiments, wherein a cluster having low abundance features relative to other clusters is de-prioritized. 163. The method of embodiment 160, or any of the above embodiments, wherein selecting comprises prioritizing a cluster having all three of a ms1p of greater than 0.33, an abundance value of greater than a signal to noise ratio of 1/10, and a low_mass_contamination and well_ratio of less than 1.164. The method of embodiment 160, or any of the above embodiments, wherein selecting comprises prioritizing a cluster having at least two of a ms1p of greater than 0.33, an abundance value of greater than 2000, and a low_mass_contamination and well_ratio of less than 1.165. The method of embodiment 160, or any of the above embodiments, wherein selecting comprises prioritizing a cluster having at least one of a ms1p of greater than 0.33, an abundance value of greater than 2000, and a low_mass_contamination and well_ratio of less than 1.166. The method of embodiment 160, or any of the above embodiments, wherein selecting comprises prioritizing a feature having a z=2 unless another feature has greater than twice its abundance. 167. The method of embodiment 160, or any of the above embodiments, wherein selecting comprises selecting 1 feature per time interval of the mass spectrometric output. 168. The method of embodiment 167, or any of the above embodiments, wherein the time interval is no greater than 2 seconds. 169. The method of embodiment 167, or any of the above embodiments, wherein the time interval is about 1.75 seconds. 170. The method of embodiment 167, or any of the above embodiments, wherein the time interval is 1.75 seconds. 171. A method of sequential mass spectrometric data analysis, comprising: receiving a first mass spectrometric output and a second mass spectrometric output; performing a quality analysis on the first mass spectrometric output; incorporating the first mass spectrometric output into a processed dataset; performing a quality analysis on the second mass spectrometric output; incorporating the second mass spectrometric output into the processed dataset; wherein performing the quality analysis on the first mass spectrometric output and receiving the second mass spectrometric output are concurrent.

### Further Discussion of Some Accompanying Figures

Turning to FIG. 1, one sees an end-to-end mass proteomics workflow of the type improved by the methods and computer systems disclosed herein. Starting from upper left, a sample such as a blood sample, or even a dried blood sample spotted onto a surface or volume (not shown) to facilitate storage and transport, is collected and optionally subjected to a quality control analysis.

For some assays such as protein based assays, a sample may be subjected to de-lipidation and abundant protein immunodepletion so as to clear constituents that may complicate quantification of proteins or other biomolecules of interest. Samples are optionally subjected to intact protein fractionation so as to assess protein content and confirm sample integrity.

Samples are processed for mass spectrometric visualization, for example via nonenzymatic or enzymatic digestion, such as TFE/trypsin digestion, as shown. Digested samples are volatilized and subjected to mass spectrometric quantification, such as LCMS, MALDI-TOF or other mass spectrometric analysis, and the outputs are quantified.

Mass spectrometric outputs are subjected to quality control assessment and quantification using any number of methods or computer systems as disclosed herein. Methods and computer systems herein facilitate quantification and quality control assessments without relying upon operator oversight, so as to generate a more accurate, more repeatable quantified mass spectrometric product in less time, so as to facilitate an automated mass spectrometric analysis workflow.

Quantified feature detection data is subjected to classifier analysis, as shown, and to identify features informative of a sample condition or status. The identified features are assembled into one or more than one biomarker panels indicative of a condition in an individual sample source.

Alternately or in combination, sample outputs are assayed as to determine levels of constituents, such as a targeted or untargeted subset of the total biomarkers in the sample. The individual source of the sample is then categorized as having a certain status for a condition for which the panel is informative. Alternately, the individual source of the sample is then categorized as having a certain percentile status relative to a reference population for the condition, so that the individual is placed relative to the reference population for the condition.

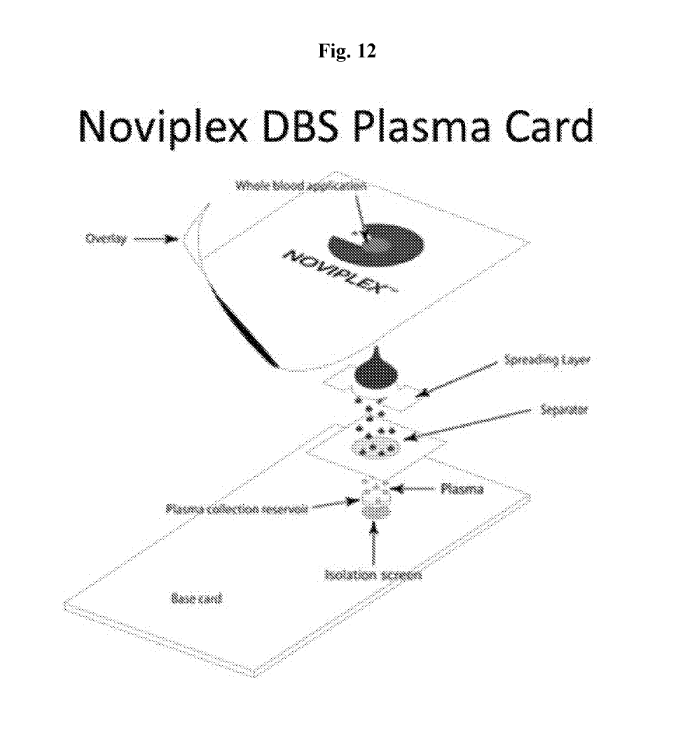

At FIG. 12 one sees an exemplary Noviplex DBS plasma card having an overlay, a spreading layer, a separator, a plasma collection reservoir, an isolation screen, and a base card. Whole blood is applied to a spot on the overlay where it reaches the spreading layer and the separator which allows the plasma to pass through to the plasma collection reservoir.

At FIG. 13 one sees 48 mass spectrometry output graphs resulting from 16 samples subjected to three mass spectrometry runs. MS1 data images from 48 injections of a technical replicate variability study are presented. The 16 DBS cards are shown in the columns with their technical replicates in the rows. For each individual MS1 image, the horizontal axis is m/z and the vertical axis is LC time. To show a high-level view of the data quality and reproducibility, a visual representation of the MS1 data from a repeated sampling experiment is shown. Here, each image in the grid shows the data from a single injection on LC time vs. m/z axes, with the color scale representing signal abundance (from black—no signal, to red—high signal). The consistency of the images shows the repeatability of the assay.

At FIG. 14 left panel one sees within card coefficients of variation (CV) with the CV on the Y axis and each DBS card on the X axis. CVs range from 3.3 to 6.2%. At FIG. 14 right panel one sees between card CV with the density on the Y axis and the between card CV on the X axis. The median CV was found to be 9.0%. CV was calculated on 64,667 features.

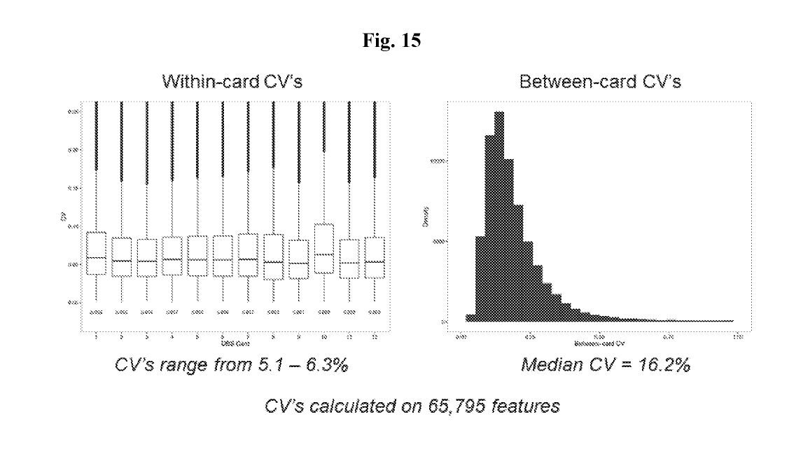

At FIG. 15 left panel one sees within card coefficients of variation (CV) with the CV on the Y axis and each DBS card on the X axis. CVs range from 5.1 to 6.3%. At FIG. 15 right panel one sees between card CV with the density on the Y axis and the between card CV on the X axis. The median CV was found to be 16.2%. CV was calculated on 65,795 features.

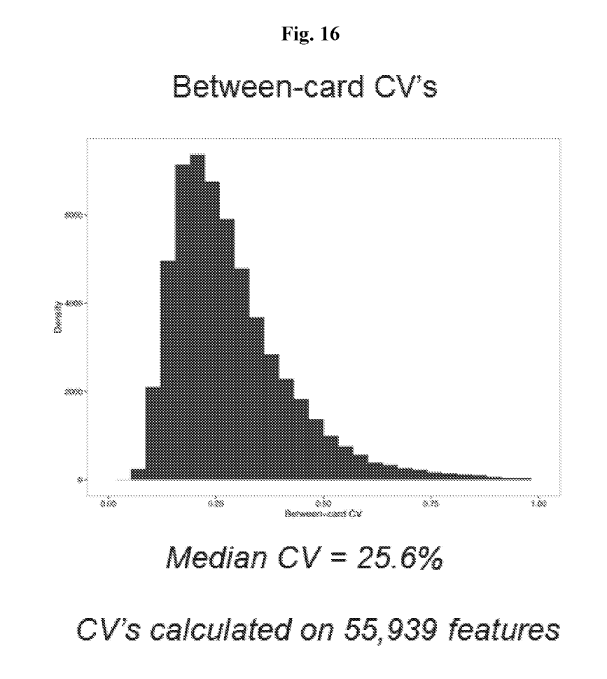

At FIG. 16, one sees between-card coefficient of variation (CV) with the density on the Y axis and the between card CV on the X axis. The median CV was 25.6% and CVs were calculated on 55,939 features.

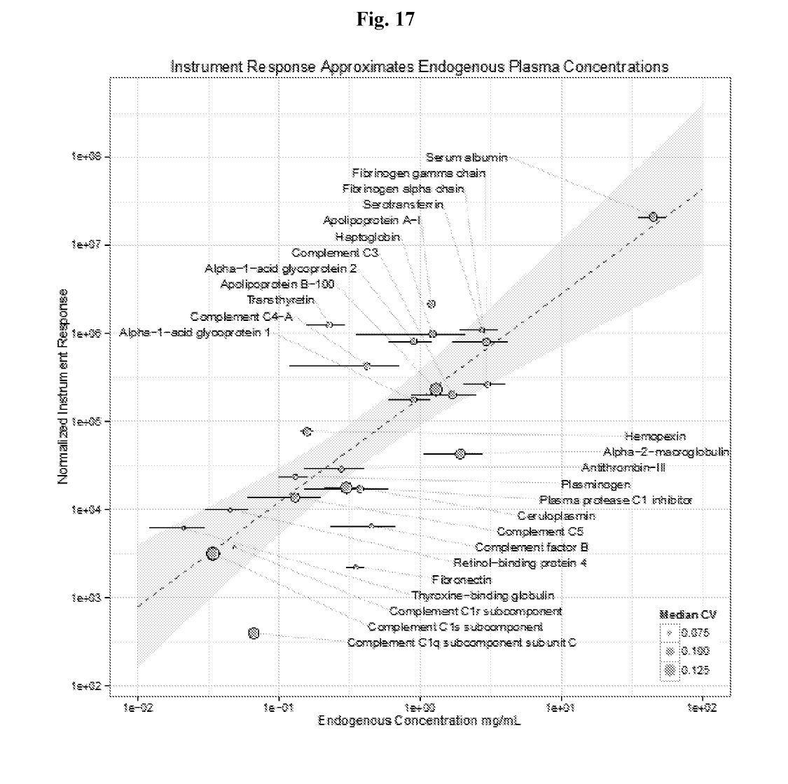

At FIG. 17 one sees a graph illustrating that instrument response is approximating endogenous plasma concentration. This graph has an X axis with the measurement of endogenous concentration and a Y axis with a normalized instrument response. Each protein is labeled with the protein name and a spot sized to the median CV with the smallest size having a median CV of 0.075, the medium size having a median CV of 0.100, and the largest size having a median CV of 0.125. A dashed line shows a perfect correlation and the shaded area shows modest variation from the perfect correlation.

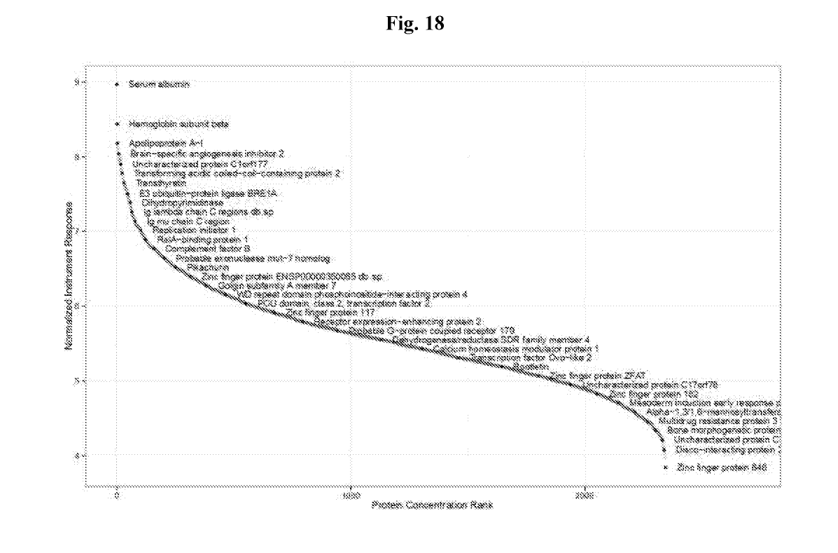

At FIG. 18 one sees a graph of the normalized instrument response versus the protein concentration rank. Proteins are ranked by protein concentration ordered on the X axis from greater to lesser concentration. The normalized instrument response is on the Y axis.

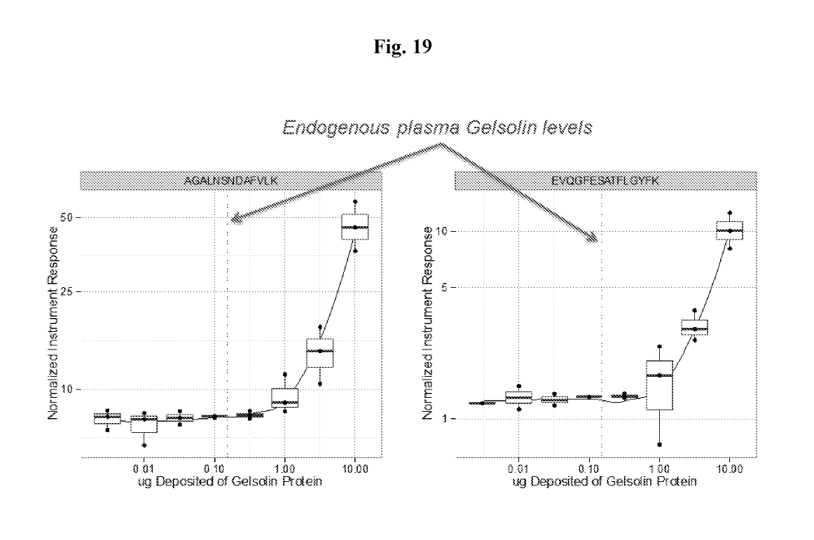

At FIG. 19 one sees endogenous plasma gelsolin levels measured using two peptides. Each graph has an X axis of μg deposited gelsolin protein and a Y axis of normalized instrument response. The left panel uses a peptide with a sequence AGALNSNDAFVLK and the right panel uses a peptide with a sequence EVQGFESATFLGYFK.

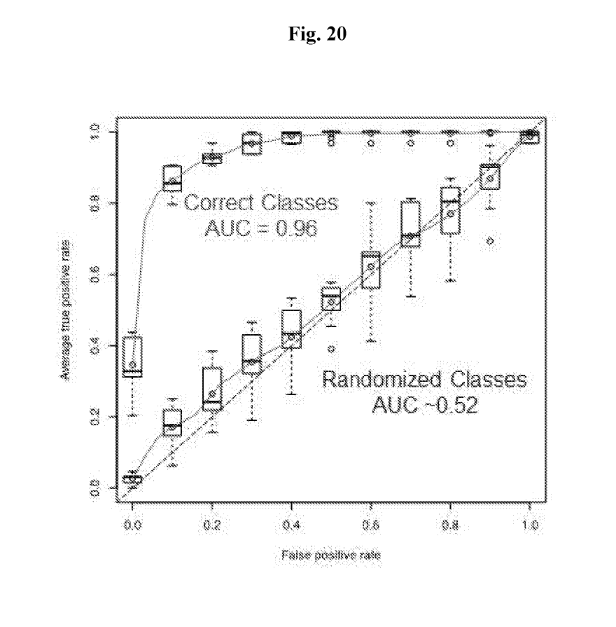

At FIG. 20 one sees the results of prediction of sex of the sample of origin. Two curves are shown on a graph with an X axis of false positive rate and a Y axis of average true positive rate. Correct classes are shown in the top curve with an AUC of 0.96 and randomized classes are shown in the bottom curve with an AUC of approximately 0.52.

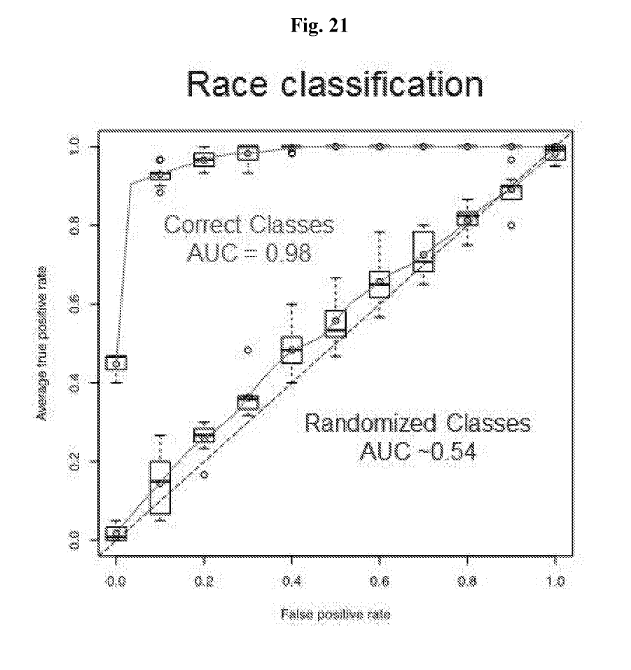

At FIG. 21 one sees the results of prediction of race of the sample of origin. Two curves are shown on a graph with an X axis of false positive rate and a Y axis of average true positive rate. Correct classes are shown in the top curve with an AUC of 0.98 and randomized classes are shown in the bottom curve with an AUC of approximately 0.54.

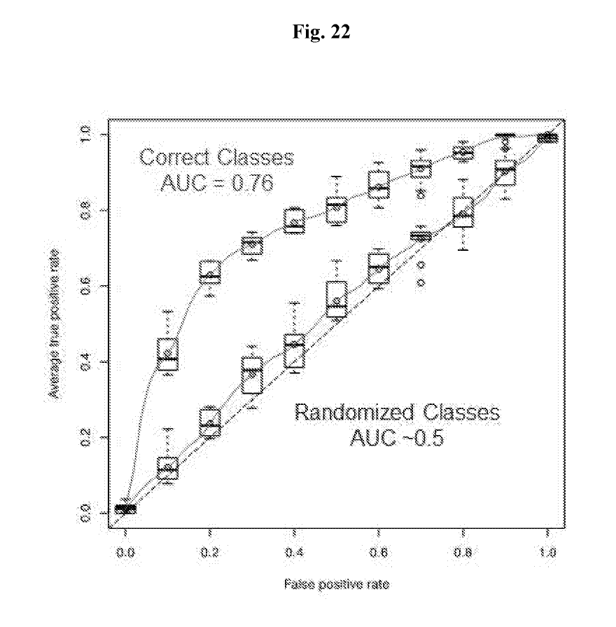

At FIG. 22 one sees the results of prediction of colorectal cancer (CRC) status of the sample of origin. Two curves are shown on a graph with an X axis of false positive rate and a Y axis of average true positive rate. Correct classes are shown in the top curve with an AUC of 0.76 and randomized classes are shown in the bottom curve with an AUC of approximately 0.5.

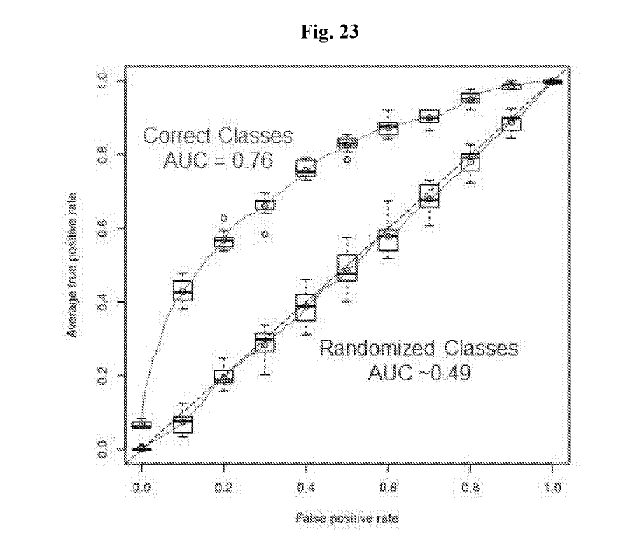

At FIG. 23 one sees the results of prediction of colorectal cancer (CRC) status of the sample of origin. Two curves are shown on a graph with an X axis of false positive rate and a Y axis of average true positive rate. Correct classes are shown in the top curve with an AUC of 0.76 and randomized classes are shown in the bottom curve with an AUC of approximately 0.49.

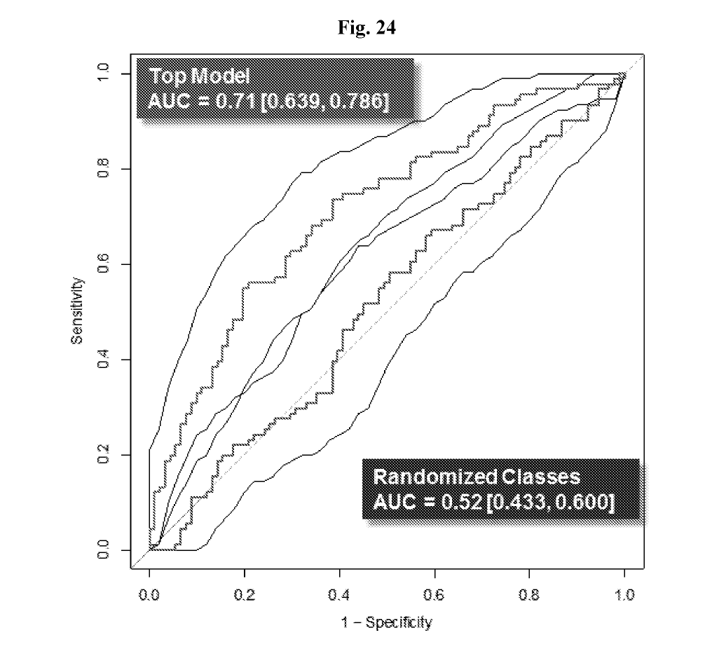

At FIG. 24 one sees the results of prediction of coronary artery disease (CAD) status of the sample of origin. Two curves are shown on a graph with an X axis of specificity and a Y axis of sensitivity. Each curve has an error curve above and below the curve. Correct classes are shown in the top curve with an AUC of 0.71 and randomized classes are shown in the bottom curve with an AUC of 0.52. One sees that the curves and their error bars do not overlap and are distinct.

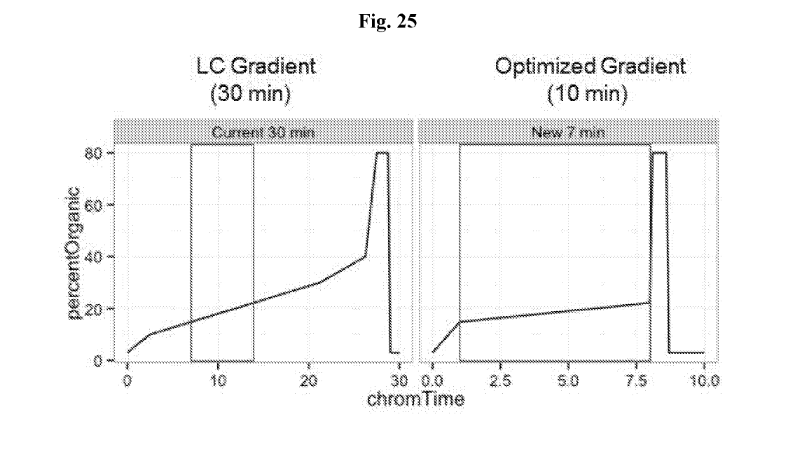

At FIG. 25 one sees two graphs of an LC gradient (left panel) and an optimized gradient (right panel. Each graph has a percent organic depicted on the Y axis and chromatography time depicted on the X axis. A linear portion of the plot is highlighted with a square.

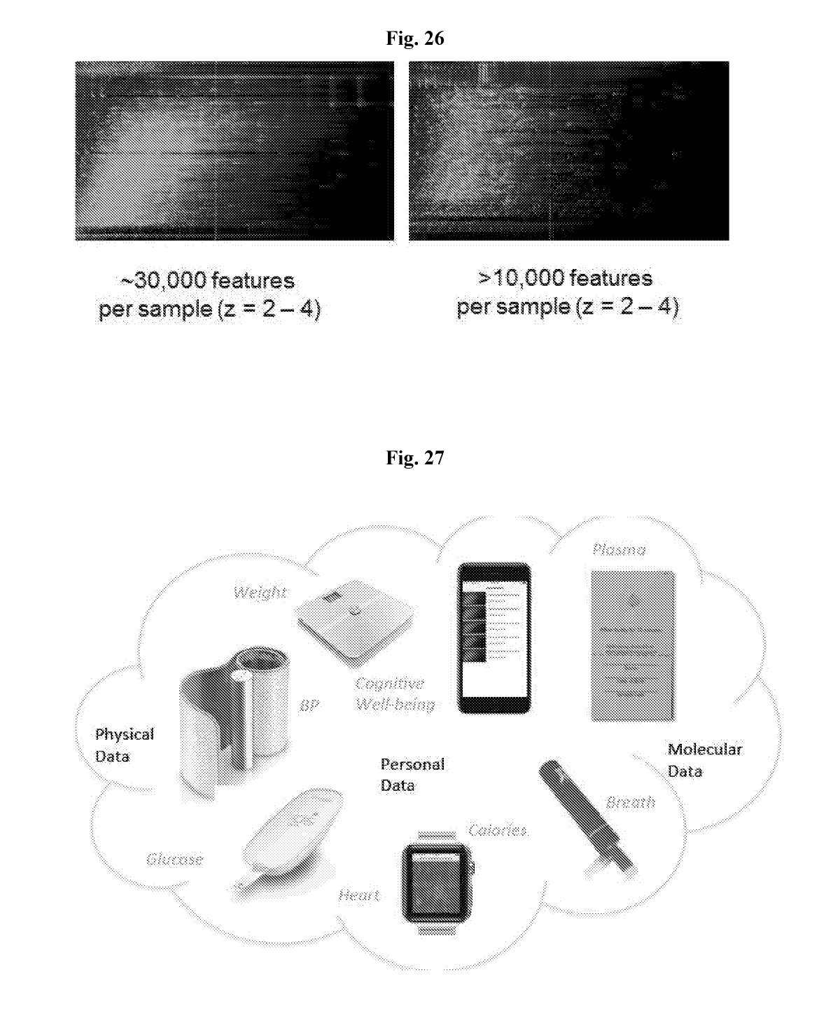

At FIG. 26 one sees a mass spectrometric analysis of a 30 minute gradient (left panel) and a 10 minute gradient (right panel). The left panel shows approximately 30,000 features per sample with a z=2-4. The right panel shows greater than 10,000 features per sample with a z=2-4.

At FIG. 27 one sees various sources of biomarker data including physical data such as blood pressure, weight, blood glucose; personal data such as cognitive well-being and heart rate; and molecular data collected from blood plasma and breath.

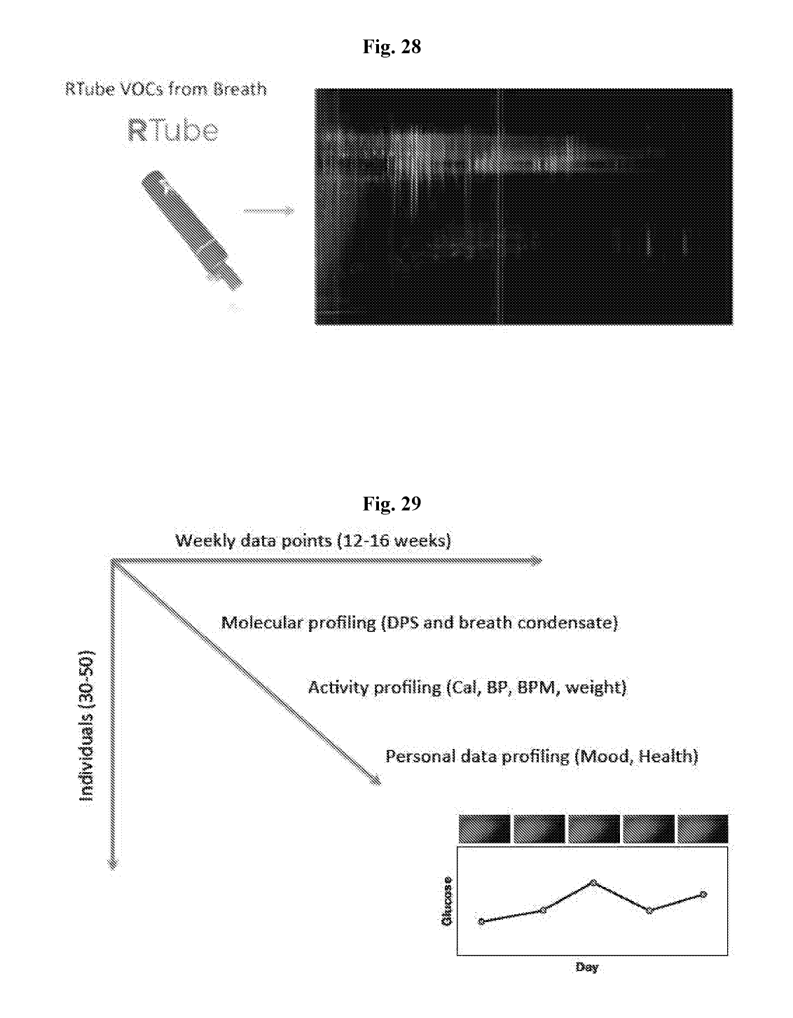

At FIG. 28 one sees an exemplary tube for collecting breath as well as VOCs analyzed by mass spectrometry from a breath sample. This figure demonstrates that meaningful biomarker data can be collected from breath.

At FIG. 29 one sees an exemplary data collection scheme of data from 30-50 individuals with data collected weekly for 12-16 weeks. Collected data include molecular profiling via DPS and breath condensate; activity profiling such as calories, blood pressure, heart rate, and weight; and personal data profiling via mood and health. These data are compiled and analyzed in an exemplary graph of blood glucose plotted each day.

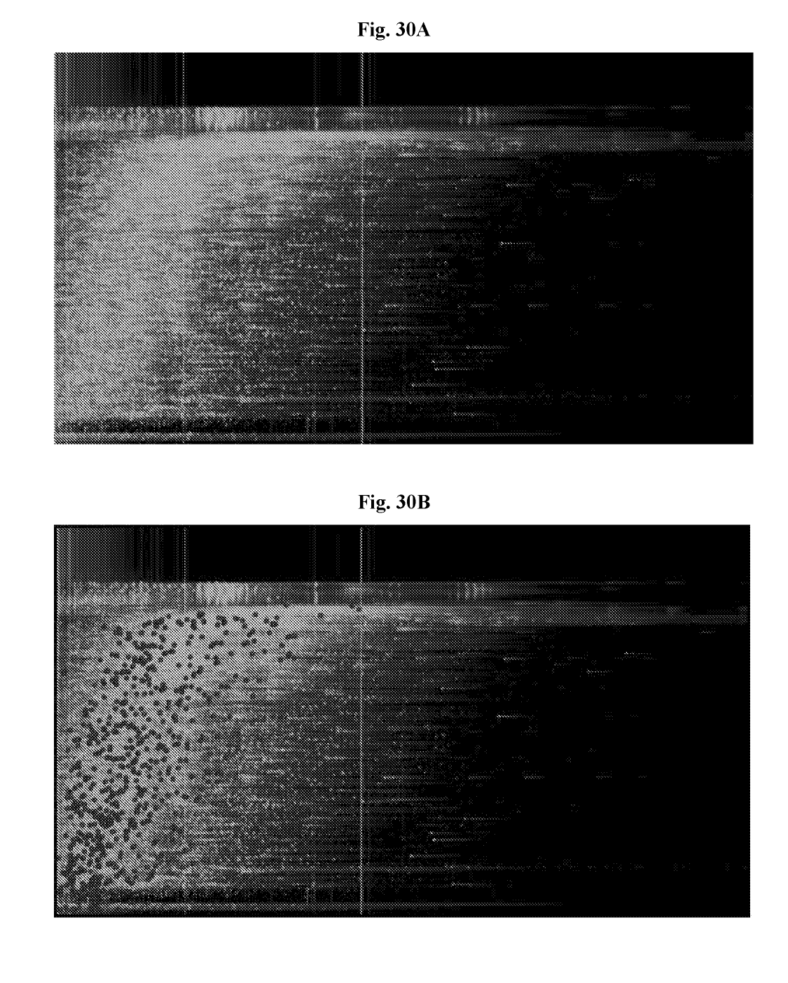

At FIG. 30A one sees output data of a mass spectrometric analysis showing more than 10,000 spots. At FIG. 30B one sees output data of a mass spectrometric analysis as in FIG. 30A with an overlay of positions of added heavy labeled markers depicted as red dots in the graph. These two figures in combination demonstrate how reference markers facilitate identification of native spots in mass spectrometric output.

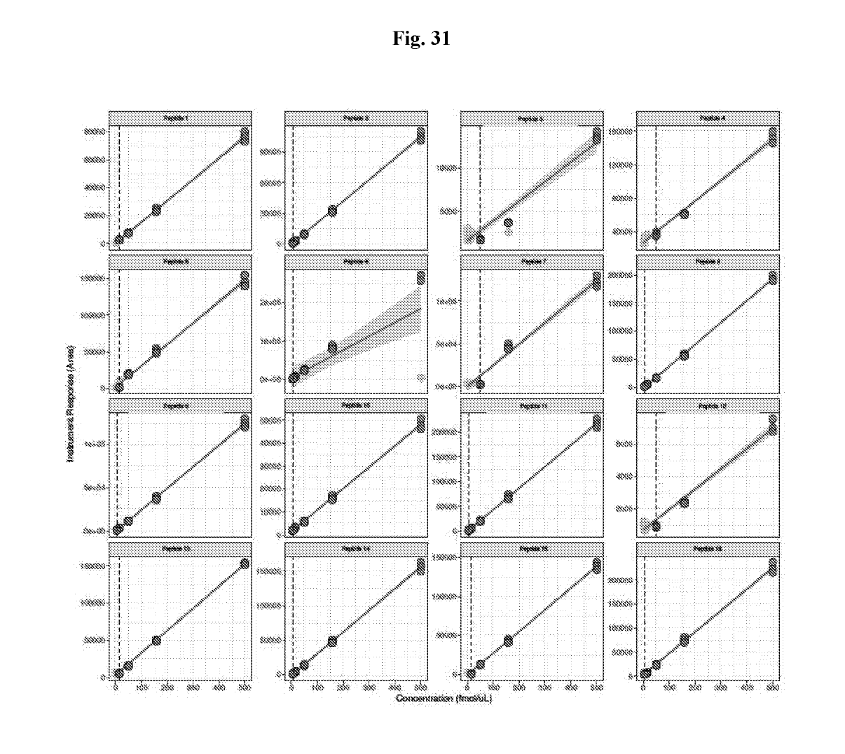

At FIG. 31 one sees results of a representative list of 16 markers. Each graph shows marker concentration on the X axis and spot signal intensity on the Y axis. Spot calls determined to be accurate are depicted as filled circles having black outlines. Spot calls determined to be miscalled are depicted as light grey without an outline.

At FIG. 32, one sees a side-by-side comparison of workflows for batch analysis (left) and concurrent analysis (right). Under a batch analysis regimen (left), a study is completed, datasets are entered completely and processed by, for example, subjecting the datasets to clustering, blank-filling and normalization, and only then is the batch integrated with previous master map data to form a new master map dataset. New data is not easily incorporated without reassessment of previously analyzed data, and processing does not occur until a study is completed.

What is claimed is:

1. A method of mass spectrometric output data processing comprising:
generating a quantified output of the mass spectrometric output;
comparing the quantified output to a reference; and
categorizing the quantified output relative to the reference,
wherein practice of the method does not require human supervision.

2. The method of claim 1, wherein a second mass spectrometric output is received concurrently with said generating a quantified output of the mass spectrometric output of a first reference.

3. The method of claim 1, wherein the method is completed in no more than 8 hours.

4. The method of claim 1, wherein the method is completed in no more than 4 hours.

5. The method of claim 1, wherein the method is completed in no more than 2 hours.

6. The method of claim 1, wherein the method is completed in no more than 1 hour.

7. The method of claim 1, wherein the method is completed in no more than 30 minutes.

8. The method of claim 1, wherein the method is completed in no more than 5 minutes.

9. The method of claim 1, wherein the method is completed in no more than 1 minute.

10. The method of claim 1, comprising obtaining a fluid sample, and subjecting the fluid sample to mass spectrometric analysis, thereby generating a quantified output of the mass spectrometric analysis.

11. The method of claim 10, wherein the fluid sample is a dried fluid sample.

12. The method of claim 11, wherein obtaining the dried fluid sample comprises depositing a sample onto a sample collection backing.

13. The method of claim 10, wherein separating plasma from whole blood on the backing.

14. The method of claim 1, wherein subjecting the dried fluid sample to mass spectrometric analysis comprises volatilizing the sample.

15. The method of claim 11, wherein subjecting the dried fluid sample to mass spectrometric analysis comprises subjecting the sample to proteolytic degradation.

16. The method of claim 15, wherein the proteolytic degradation comprises enzymatic degradation.

17. The method of claim 16, wherein the enzymatic degradation comprises contacting a sample to at least one of ArgC, AspN, chymotrypsin, GluC, LysC, LysN, trypsin, snake venom diesterase, pectinase, papain, alcanase, neutrase, snailase, cellulase, amylase, and chitinase.

18. The method of claim 16, wherein the enzymatic degradation comprises trypsin degradation.

19. The method of claim 15, wherein the proteolytic degradation comprises nonenzymatic degradation.

20. The method of claim 19, wherein the nonenzymatic degradation comprises at least one of heat, acidic treatment, and salt treatment.

21. The method of claim 19, wherein the nonenzymatic degradation comprises contacting a sample to at least one of hydrochloric acid, formic acid, acetic acid, hydroxide bases, cyanogen bromide, 2-nitro-5-thiocyanobenzoate, and hydroxylamine.

22. The method of any one of claims 1-20, wherein generating a quantified output of the mass spectrometric analysis comprises quantifying at least 20 mass points.

23. The method of any one of claims 1-20, wherein generating a quantified output of the mass spectrometric analysis comprises quantifying at least 50 mass points.

24. The method of any one of claims 1-20, wherein generating a quantified output of the mass spectrometric analysis comprises quantifying at least 100 mass points.

25. The method of any one of claims 1-20, wherein generating a quantified output of the mass spectrometric analysis comprises quantifying at least 5,000 mass points.

26. The method of any one of claims 1-20, wherein generating a quantified output of the mass spectrometric analysis comprises quantifying at least 15,000 mass points

27. The method of any one of claims 1-20, wherein generating a quantified output of the mass spectrometric analysis is completed in no more than 30 minutes.

28. The method of any one of claims 1-20, wherein generating a quantified output of the mass spectrometric analysis is completed in no more than 15 minutes.

29. The method of any one of claims 1-20, wherein generating a quantified output of the mass spectrometric analysis is completed in no more than 10 minutes.

30. The method of any one of claims 1-20, wherein generating a quantified output of the mass spectrometric analysis is completed in no more than 5 minutes.

31. The method of any one of claims 1-20, wherein generating a quantified output of the mass spectrometric analysis is completed in no more than 1 minute.

32. The method of any one of claims 1-20, wherein generating a quantified output of the mass spectrometric analysis is automated.

33. The method of any one of claims 1-20, wherein generating a quantified output of the mass spectrometric analysis comprises generating an adjusted abundance value.

34. The method of any one of claims 1-20, wherein generating a quantified output of the mass spectrometric analysis comprises generating an adjusted mz value.

35. The method of any one of claims 1-20, wherein generating a quantified output of the mass spectrometric analysis comprises performing a convolution operation to reduce pixel-by-pixel noise of the mass spectrometry data; and identifying a plurality of features of the sample, wherein identifying the plurality of features comprises identifying a plurality of peaks of the mass spectrometry data, and determining a respective mz value and a respective LC value for the plurality of peaks.

36. The method of any one of claims 1-20, wherein generating a quantified output of the mass spectrometric analysis comprises receiving data for a plurality of identified peaks from mass spectrometry data of the sample; filtering the plurality of identified peaks to provide a filtered set of peaks, the filtering comprising (1) a first filtering process on the data for the plurality of identified peaks, the first filtering process comprising a peak contrast filtering process, and (2) a second filtering process for removing at least one of a spurious peak and a peak corresponding a calibrant analyte; and selecting a subset of peaks from the plurality of peaks, the subset of peaks comprising peaks corresponding to molecular feature isotopic clusters.

37. The method of any one of claims 1-20, wherein generating a quantified output of the mass spectrometric analysis comprises receiving mass spectrometry data of the sample, the mass spectrometry data comprising data for a peptide; and determining a metric value indicative of a likelihood of successful sequencing of the peptide.

38. The method of any one of claims 1-20, wherein generating a quantified output of the mass spectrometric analysis comprises receiving mass spectrometry data of the sample, the mass spectrometry data comprising a molecular mass value of the sample; and determining, using the mass defect histogram library, a mass defect probability for identifying the molecular mass value, wherein the mass defect probability is indicative of a probability the molecular mass value corresponds to a peptide from the sample.

39. The method of any one of claims 1-21, wherein generating a quantified output of the mass spectrometric analysis comprises receiving tandem mass spectrometry data of the sample, the tandem mass spectrometry data comprising respective molecular mass values for a plurality of identified peaks; and determining a metric value indicative of correspondence between the molecular mass values and molecular mass values of known peptides fragments.

40. The method of any one of claims 1-21, wherein generating a quantified output of the mass spectrometric analysis comprises receiving tandem mass spectrometry data of the sample, the tandem mass spectrometry data comprising respective molecular mass values for a plurality of identified peaks; and determining a metric value indicative of correspondence between the molecular mass values and molecular mass values of known peptides.

41. The method of any one of claims 1-21, wherein generating a quantified output of the mass spectrometric analysis comprises identifying data features corresponding to the set of targeted mass spectrometric features; determining characteristics comprising mass, charge and elution time for the data features; and calculating deviation between targeted mass spectrometric feature characteristics and data feature characteristic.

42. The method of any one of claims 1-21, wherein generating a quantified output of the mass spectrometric analysis comprises comparing mass spectrometry data to the set of protein modifications and digestion variants; and assessing the frequency of at least one of protein modifications and digestion frequency.

43. The method of any one of claims 1-21, wherein generating a quantified output of the mass spectrometric analysis comprises identifying test peptide signals in a mass spectrometric output.

44. The method of any one of claims 1-21, wherein generating a quantified output of the mass spectrometric analysis comprises identifying reference clusters having exactly one feature per sample; assigning an index area derived from the reference clusters; and mapping nonreference clusters onto the index area.

45. The method of any one of claims 1-21, wherein generating a quantified output of the mass spectrometric analysis comprises identifying features having common m/z ratios across a plurality of samples; aligning said features across a plurality of samples; bringing LC times for said features in line; and clustering said features.

46. The method of any one of claims 1-21, wherein generating a quantified output of the mass spectrometric analysis comprises identifying features having common m/z ratios and common LC times across a plurality of fractions of a sample; assigning to a common cluster features sharing a common m/z ratios and common LC times in adjacent fractions; and discarding said cluster and retain said features when said cluster has at least one of a size above a threshold and an LC time above a threshold.

47. The method of any one of claims 1-21, wherein generating a quantified output of the mass spectrometric analysis comprises choosing a first random subset of fraction outputs; counting the number of unique pieces of information for the first random subset of fraction outputs; choosing a second random subset of fraction outputs; counting the number of unique pieces of information for the second random subset of fraction outputs; and selecting the random subset of fraction outputs having the greater number of unique pieces of information.

48. The method of any one of claims 1-21, wherein generating a quantified output of the mass spectrometric analysis comprises identifying measured features for said mass spectrometric fraction outputs; calculating average m/z and LC time values for measured features appearing in multiple mass spectrometric fraction outputs; assaying for unidentified features sharing at least one of average m/z and LC time values with said measured features; and assigning at least one of said unidentified features to a cluster of a measured feature, so as to generate at least one inferred mass feature.

49. The method of any one of claims 1-21, wherein generating a quantified output of the mass spectrometric analysis comprises calculating expected LC retention times; calculating standard deviation values of expected LC retention times; comparing expected LC retention times to observed associated LC retention times; and discarding mass spectrometric peptide identification calls for which expected LC retention times differ from observed associated LC retention times by more than standard deviation values.

50. The method of any one of claims 1-21, wherein generating a quantified output of the mass spectrometric analysis comprises identifying features corresponding to common peptides and having differing LC retention times in the plurality of mass spectrometry outputs; applying an LC retention time shift to one of the mass spectrometry outputs so as to bring the differing LC times into closer alignment for the features corresponding to common peptides; applying the LC retention time shift to additional features in proximity to the features corresponding to common peptides in the mass spectrometry output; and discarding mass spectrometric peptide identification calls for which expected LC retention times differ from observed associated LC retention times by more than standard deviation values.

51. The method of any one of claims 1-21, wherein generating a quantified output of the mass spectrometric analysis comprises grouping proteins sharing at least one common peptide; determining a minimum number of proteins per group; and determining a sum for the minimum number of proteins per group for all groups.

52. The method of any one of claims 1-21, wherein generating a quantified output of the mass spectrometric analysis comprises constructing a command line in a format compatible with a given search engine; initiating execution of the search engine; parsing the search engine output; and configuring the output into a standard format.

53. The method of any one of claims 1-21, wherein generating a quantified output of the mass spectrometric analysis comprises parsing file contents from a memory unit into key-value pairs; read each key-value pair into a standard format; and writing the standard format key-value pairs into an output file.

54. The method of any one of claims 1-21, wherein generating a quantified output of the mass spectrometric analysis comprises parsing a file into an array of key-value pairs representative of tandem mass spectra and corresponding attributes; obtaining corresponding precursor ion attributes; replacing mass spectrum file values using precursor ion attributes when precursor ion attributes are indicated to be accurate; and configuring the file into a flat format output.

55. The method of any one of claims 1-21, wherein generating a quantified output of the mass spectrometric analysis comprises receiving a mass spectrometry output having a plurality of unidentified features; including features having a z-value of greater than 1 up to and including 5; clustering included features by retention time to form clusters; de-prioritizing clusters for which verification has been previously performed; selecting a single feature per cluster; and verifying a feature of at least one cluster.

56. The method of any one of claims 1-21, wherein generating a quantified output of the mass spectrometric analysis comprises generating a processed dataset from one of a plurality of received mass spectrometric output; and incorporating the processed dataset into a processed study dataset.

57. The method of any one of claims 1-21, wherein generating a quantified output of the mass spectrometric analysis comprises receiving a first mass spectrometric output and a second mass spectrometric output; performing a quality analysis on the first mass spectrometric output; incorporating the first mass spectrometric output into a processed dataset; performing a quality analysis on the second mass spectrometric output; incorporating the second mass spectrometric output into the processed dataset; wherein performing the quality analysis on the first mass spectrometric output and receiving the second mass spectrometric output are concurrent.

58. The method of any one of claims 1-21, wherein generating a quantified output of the mass spectrometric analysis does not comprise human analysis of the mass spectrometric analysis.

59. The method of any one of claims 1-21, wherein generating a quantified output of the mass spectrometric analysis comprises identifying at least 3 reference mass outputs in the mass spectrometric analysis.

60. The method of any one of claims 1-21, wherein generating a quantified output of the mass spectrometric analysis comprises identifying at least 6 reference mass outputs in the mass spectrometric analysis.

61. The method of any one of claims 1-21, wherein generating a quantified output of the mass spectrometric analysis comprises identifying at least 10 reference mass outputs in the mass spectrometric analysis.

62. The method of any one of claims 1-21, wherein generating a quantified output of the mass spectrometric analysis comprises identifying at least 100 reference mass outputs in the mass spectrometric analysis.

63. The method of claim 59, wherein the at least 3 reference mass outputs are introduced to the sample prior to analysis.

64. The method of claim 59, wherein the at least 3 reference mass outputs differ from sample mass outputs by known amounts.

65. The method of claim 59, wherein the at least 3 reference mass outputs have known amounts.

66. The method of claim 65, comprising comparing reference mass output amounts to sample output amounts.

67. The method of claim 1, wherein comparing the quantified output to a reference comprises identifying a subset of the sample mass output, and comparing said subset of the sample mass output to the reference.

68. The method of claim 1, wherein the reference comprises at least one sample output of known status for a health category.

69. The method of claim 1, wherein the reference comprises at least ten sample outputs of known status for a health category.

70. The method of claim 1, wherein the reference comprises at least ten samples of unknown health status for a health category.

71. The method of claim 1, wherein the reference comprises predicted values for a health status for a health category.

72. The method of claim 1, wherein the reference comprises samples taken from at least two individuals.

73. The method of claim 1, wherein the reference comprises samples taken from at least two time points.

74. The method of claim 1, wherein the reference comprises a sample taken from a source common to the sample.

75. The method of claim 1, wherein categorizing the quantified output relative to the reference comprises assigning a health category status to an individual source of the sample.

76. The method of claim 1, wherein categorizing the quantified output relative to the reference comprises assigning the reference health category status to an individual source of the sample.

77. The method of claim 1, wherein categorizing the quantified output relative to the reference comprises assigning the reference health category status to an individual source of the sample.

78. The method of claim 1, wherein categorizing the quantified output relative to the reference comprises assigning a percentage value to an individual source of the sample.

79. The method of claim 78, wherein the percentage value represents the position of the sample relative to the reference.
